(19) United States
(12) Patent Application Publication
(10) Pub. No.: US 2024/0165160 A1
(43) Pub. Date: May 23, 2024

(54) EFFICACY AND DURABLE RESPONSE OF IMMUNOTHERAPY

(71) Applicant: Kite Pharma, Inc., Foster City, CA (US)

(72) Inventors: Justin A. Budka, Fishers, IN (US); Justyna E. Kanska, Burbank, CA (US)

(21) Appl. No.: 18/495,531

(22) Filed: Oct. 26, 2023

Related U.S. Application Data
(60) Provisional application No. 63/515,492, filed on Jul. 25, 2023.
(60) Provisional application No. 63/479,877, filed on Jan. 13, 2023.
(60) Provisional application No. 63/386,457, filed on Dec. 7, 2022.
(60) Provisional application No. 63/381,525, filed on Oct. 28, 2022.

(51) Int. Cl. A61K 35/17; A61K 39/00; A61P 35/00; C07K 16/28; C12N 5/0783; C12N 15/86; G01N 33/50

(52) U.S. Cl. CPC A61K 35/17; A61K 39/4611; A61K 39/4631; A61K 39/464412; A61P 35/00; C07K 16/2803; C12N 5/0636; C12N 15/86; G01N 33/505; A61K 2239/13; A61K 2239/38; A61K 2239/48; G01N 2800/52

(57) ABSTRACT

Provided herein are methods for preparing, producing, processing, culturing, isolating, of making cells suitable for immune or cell therapy, and for their use in cell therapy. Further provided are methods for treatment of a cancer patient with such cells.

### CROSS-REFERENCES TO RELATED APPLICATIONS

This application claims the benefit of priority to U.S. Provisional Patent Application No. 63/381,525, filed 28 Oct. 2022 and titled “Efficacy and Durable Response of Immunotherapy”, U.S. Provisional Patent Application No. 63/386,457, filed 7 Dec. 2022 and titled “Efficacy and Durable Response of Immunotherapy”. U.S. Provisional Patent Application No. 63/479,877, filed 13 Jan. 2023 and titled “Efficacy and Durable Response of Immunotherapy”, and U.S. Provisional Patent Application No. 63/515,492, filed 25 Jul. 2023 and titled “Efficacy and Durable Response of Immunotherapy” the entireties of which are incorporated by reference herein.

### FIELD

The present application relates to CAR-T cells, methods of making them, and methods of using them to treat cancer.

### BACKGROUND

Human cancers are by their nature comprised of normal cells that have undergone a genetic or epigenetic conversion to become abnormal cancer cells. Cancer cells express proteins and other antigens that are distinct from those expressed by normal cells. These aberrant tumor antigens may be used by the body's innate immune system to specifically target and kill cancer cells. However, cancer cells employ various mechanisms to prevent immune cells, such as T and B lymphocytes, from successfully targeting cancer cells. Human T cell therapies rely on ex-vivo, enriched or modified human T cells to target and kill cancer cells in a subject, e.g., a patient. Various technologies have been developed to prepare T cell populations with enriched concentrations of naturally occurring T cells capable of targeting a tumor antigen, remove circulating tumor cells, and/or genetically modifying T cells to specifically target a known cancer antigen, thus producing populations of chimeric antigen receptor (CAR)-T cells for cancer therapy. Some of these therapies have shown promising effects on tumor size and patient survival. There exists a need for therapeutic methods utilizing (CAR)-T cells for cancer therapy, including methods comprising bridging therapies.

### SUMMARY

Any aspect or embodiment described herein may be combined with any other aspect or embodiment as disclosed herein. While the present invention has been described in conjunction with the detailed description thereof, the description is intended to illustrate and not limit the scope of the present invention, which is partially defined by the scope of the appended claims. Other aspects, advantages, and modifications are within the scope of the following embodiments/claims.

1. A method for treating mantle cell lymphoma (MCL) or B cell ALL in a subject in need thereof comprising administering to the subject a therapeutically effective amount of a T cell product comprising autologous T cells expressing an anti-CD19 chimeric antigen receptor (CAR), wherein the MCL or B cell ALL is relapsed or refractory MCL following one or more prior treatment selected from the group consisting of chemotherapy, radiotherapy, immunotherapy, an autologous stem cell transplant (SCT), or any combination thereof, further wherein the one or more prior treatment does not comprise a Bruton Tyrosine Kinase inhibitor (BTKi).

2. The method of aspect 1, wherein the subject has received 1-5 prior treatments, wherein at least one of the prior treatments is selected from autologous SCT, anti-CD20 antibody, and/or anthracycline- or bendamustine-containing chemotherapy.

3. The method of aspect 1 or 2, wherein the BTKi is ibrutinib or acalabrutinib.

4. The method of any one of aspects 1 through 3, wherein R/R B cell ALL is defined as refractory to first-line therapy (i.e., primary refractory), relapsed≤12 months after first remission, relapsed or refractory after ≥2 prior lines of systemic therapy, or relapsed after allogeneic SCT, wherein the subject is required to have ≥5% bone marrow blasts, an Eastern Cooperative Oncology Group performance status of 0 or 1, and/or adequate renal, hepatic, and cardiac function.

5. The method of any one of aspects 1 through 4, wherein if the B cell ALL subject has received prior blinatumomab, the subject is required to have leukemic blasts with CD19 expression ≥90%.

6. The method of any one of aspects 1 through 5, wherein the subject receives bridging therapy after leukapheresis and before conditioning/lymphodepleting chemotherapy.

7. The method of any one of aspects 1 through 6, wherein the MCL subject receives a lymphodepleting chemotherapy regimen of cyclophosphamide 500 mg/m² intravenously and fludarabine 30 mg/m² intravenously, both given on each of the fifth, fourth, and third days before T cell infusion.

8. The method of any one of aspects 1 through 7, wherein the B cell ALL subject receives a lymphodepleting regimen of fludarabine intravenous (IV) 25 mg/m²/day on each of the fourth, third, second days before T cell infusion, and cyclophosphamide IV 900 mg/m²/day on the second day before infusion.

9. The method of any one of aspects 6 or 8, wherein the MCL bridging therapy is selected from dexamethasone (e.g., 20-40 mg or equivalent PO or IV daily for 1-4 days); methylprednisolone, ibrutinib (e.g., 560 mg PO daily), and/or acalabrutinib (e.g., 100 mg PO twice daily); an immunomodulator; R-CHOP, bendamustine; alkylating agents; and/or platinum-based agents, wherein the bridging therapy is administered after leukapheresis and completed in, for example, 5 days or less before conditioning chemotherapy.

10. The method of any one of aspects 6 through 8, wherein the B cell ALL subject may receive any one or more of the following bridging chemotherapy regimens:

Predefined Bridging Chemotherapy Regimens

| | |
|---|---|
| Attenuated | Vincristine non-liposomal (1-2 mg IV weekly) or |
| VAD | liposomal (2.25 mg/m² IV weekly), and |
| | dexamethasone 20-40 mg IV or PO daily × 3-4 |
| | days per week. Optional doxorubicin 50 mg/m² |
| | IV × 1 (first week only) |
| Mercaptopurine | 50-75 mg/m²/day by mouth (administer at bedtime |
| (6-MP) | on an empty stomach to improve absorption) |
| Hydroxyurea | Doses titrated between 15-50 mg/kg/day (rounded |
| | to the nearest 500 mg capsule and given as a single |
| | daily oral dose on a continuous basis) |
| DOMP | Dexamethasone 6 mg/m²/day PO (or IV) divided |
| | BID days 1-5, vincristine 1.5 mg/m² (maximum |
| | dose 2 mg) IV on day 1, methotrexate 20 mg/m² |
| | PO weekly, 6-MP 50-75 mg/m²/day PO daily |
| Attenuated | Fludarabine 30 mg/m² IV days 1-2, cytarabine |
| FLAG/ | 2 g/m² IV days 1-2, G-CSF 5 μg/kg SC or IV |
| FLAG-IDA | starts on day 3 and can continue until day before |
| | the start of conditioning chemotherapy. With or |
| | without idarubicin 6 mg/m² IV days 1-2 |
| Mini-hyper | Course A: Cyclophosphamide 150 mg/m² every |
| CVAD | 12 h × 3 days, dexamethasone 20 mg/d IV or PO |
| (courses A | daily days 1-4 and 11-14, vincristine 2 mg IV × 1 |
| and/or B) | Course B: methotrexate 250 mg/m² IV over |
| | 24 hours on day 1, cytarabine 0.5 g/m² IV |
| | every 12 hours × 4 doses on days 2 and 3 |

11. The method of any one of aspects 1 through 10, wherein the T cell product comprises CD4+ and CD8+ CAR T cells that are prepared from peripheral blood mononuclear cells (PBMCs) by positive enrichment and consequent partial or complete depletion of circulating cancer cells.

12. The method of aspect 11, wherein the PBMC are enriched for T cells by positive selection for CD4+ and CD8+ cells, activated with anti-CD3 and anti-CD28 antibodies in the presence of IL-2, and then transduced with a replication-incompetent viral vector containing FMC63-28Z CAR, a chimeric antigen receptor (CAR) comprising an anti-CD19 single-chain variable fragment (scFv), CD28 and CD3-zeta domains.

13. The method of aspect 11 or 12, wherein the T cell product comprises fewer cancer cells than a T cell product comprising T cells from a leukapheresis-derived product that have not been positively selected for CD4+ and CD8+ T cells.

14. The method of any one of aspects 11 through 13, wherein the T cell product has other superior product attributes relative to a T cell product comprising T cells from a leukapheresis-derived product that have not been positively selected/enriched for CD4+ and CD8+ T cells.

15. The method of aspect 14, wherein the superior product attributes are selected from increased percentage of CDRA45+CCR7+(naïve-like) T cells, decreased percentage of differentiated T cells, increased percentage of CD3+ cells, decreased IFN-gamma production, and/or decreased percentage of CD3− cells.

16. The method of any one of aspects 1 through 15, wherein the MCL subject is administered one or more doses of 1.8×10⁶, 1.9×10⁶, or 2×10⁶ CAR positive viable T cells per kg body weight, with a maximum of 2×10⁸ CAR positive viable T cells (for patients 100 kg and above) and the B cell ALL subject is administered 0.5×10⁶, 1×10⁶, or 2×10⁶ CAR positive viable T cells per kg body weight, with a maximum of 2×10⁸ CAR positive viable T cells (for patients 100 kg and above).

17. The method of any one of aspects 1 through 15, wherein if the subject has achieved complete response to the first infusion, the subject may receive a second infusion of anti-CD19 CAR T cells, if progressing following >3 months of remission, provided CD19 expression has been retained and neutralizing antibodies against the CAR are not suspected, wherein response is assessed using the Lugano classification.

18. The method of any one of aspects 1 through 17, wherein the subject is monitored for signs and symptoms of cytokine release syndrome (CRS) and neurologic toxicity after T cell administration.

19. The method of aspect 18, wherein the subject is monitored daily for at least seven days, preferably for four weeks, following infusion for signs and symptoms of CRS and neurologic toxicity.

20. The method of aspect 18 or 19, wherein the signs or symptoms associated with CRS comprise fever, chills, fatigue, tachycardia, nausea, hypoxia, and/or hypotension and the signs or symptoms associated with neurologic events comprise encephalopathy, seizures, changes in level of consciousness, speech disorders, tremors, and/or confusion.

21. The method of any one of aspects 18 through 20, wherein cytokine release syndrome in MCL subjects is managed in accordance with the following protocol:

| CRS Grade | Tocilizumab | Corticosteroids |
|---|---|---|
| Grade 1 | If not improving after 24 | Not applicable. |
| Symptoms require | hours, administer tocilizumab | |
| symptomatic treatment only | 8 mg/kg intravenously over 1 | |
| (e.g., fever, nausea, fatigue, | hour (not to exceed 800 mg). | |
| headache, myalgia, malaise). | | |
| Grade 2 | Administer tocilizumab | Manage per Grade 3 |
| Symptoms require and | 8 mg/kg intravenously over 1 | if no improvement |
| respond to moderate | hour (not to exceed 800 mg). | within 24 hours after |
| intervention. | Repeat tocilizumab every 8 | starting tocilizumab. |
| Oxygen requirement less than | hours as needed if not | If improving, taper |
| 40% FiO2 or hypotension | responsive to intravenous fluids | corticosteroids. |
| responsive to fluids or low | or increasing supplemental | |
| dose of one vasopressor or | oxygen. Limit to a maximum of | |
| Grade 2 organ toxicity. | 3 doses in a 24-hour period; | |
| | maximum total of 4 doses if no | |
| | clinical improvement in the | |
| | signs and symptoms of CRS. | |
| | If improving, discontinue | |
| | tocilizumab. | |
| Grade 3 | Per Grade 2 | Administer |
| Symptoms require and | | methylprednisolone |
| respond to aggressive | | 1 mg/kg intravenously |
| intervention. | | twice daily or |
| Oxygen requirement greater | | equivalent |
| than or equal to 40% FiO2 or | | dexamethasone (e.g., |
| hypotension requiring high- | | 10 mg intravenously |
| dose or multiple vasopressors | | every 6 hours) until |
| or Grade 3 organ toxicity or | | Grade 1, then taper |
| Grade 4 transaminitis. | | corticosteroids. |
| | | If improving, manage |
| | | as Grade 2. |
| | | If not improving, |
| | | manage as Grade 4. |
| Grade 4 | Per Grade 2 | Administer |
| Life-threatening symptoms. | | methylprednisolone |
| Requirements for ventilator | | 1000 mg intravenously |
| support or continuous | | per day for 3 days. |
| veno-venous hemodialysis | | If improving, taper |
| (CVVHD), or Grade 4 organ | | corticosteroids, and |
| toxicity (excluding | | manage as Grade 3. |
| transaminitis). | | If not improving, |
| | | consider alternate |
| | | immunosuppressants. |

22. The method of any one of aspects 18 through 21, wherein neurologic toxicity in MCL subjects is managed in accordance with the following protocol:

| Grading | | |
|---|---|---|
| Assessment | Concurrent CRS | No Concurrent CRS |
| Grade 2 | Administer tocilizumab per claim | Administer dexamethasone 10 mg |
| | 15 for management of Grade 2 CRS. | intravenously every 6 hours until |
| | If not improving within 24 hours after | the event is Grade 1 or less, then |
| | starting tocilizumab, administer | taper corticosteroids. |
| | dexamethasone 10 mg intravenously | |
| | every 6 hours until the event is Grade 1 | |
| | or less, then taper corticosteroids. | |
| | If still not improving, manage as Grade 3. | |
| | Consider non-sedating anti-seizure medicines | |
| | (e.g., levetiracetam) for seizure prophylaxis. | |
| Grade 3 | Administer tocilizumab per claim | Administer dexamethasone 10 mg |
| | 15 for management of Grade 2 CRS. | intravenously every 6 hours. |
| | In addition, administer dexamethasone | Continue dexamethasone use |
| | 10 mg intravenously with the first dose of | until the event is Grade 1 or |
| | tocilizumab and repeat dexamethasone | less, then taper corticosteroids. |
| | dose every 6 hours. Continue | If not improving, manage as |
| | dexamethasone use until the event is | Grade 4. |
| | Grade 1 or less, then taper corticosteroids. | |
| | If improving, discontinue tocilizumab | |
| | and manage as Grade 2. | |
| | If still not improving, manage as | |
| | Grade 4 (below). | |
| | Consider non-sedating anti-seizure medicines | |
| | (e.g., levetiracetam) for seizure prophylaxis. | |
| Grade 4 | Administer tocilizumab per claim | Administer methylprednisolone |
| | 15 for management of Grade 2 CRS. | 1000 mg intravenously per day |
| | Administer methylprednisolone 1000 mg | for 3 days. |
| | intravenously per day with first dose of | If improving, then manage as |
| | tocilizumab and continue | Grade 3. |
| | methylprednisolone 1000 mg | If not improving, consider |
| | intravenously per day for 2 more days. | alternate immunosuppressants. |
| | If improving, then manage as Grade 3. | |
| | If not improving, consider | |
| | alternate immunosuppressants. | |

Consider non-sedating anti-seizure medicines
(e.g., levetiracetam) for seizure prophylaxis.

23. The method of any one of aspects 1 through 22, wherein the MCL subjcet is a high-risk patient as determined by a Ki-67 tumor proliferation index≥50% and/or presence of a TP53 mutation.

24. The method of any one of aspects 18 through 20, wherein CRS in a B cell ALL subject is managed according to the following protocol:

| CRS Grade | Tocilizumab | Corticosteroids |
|---|---|---|
| Grade 1 | If not improving after 24 | Not applicable. |
| Symptoms require | hours, administer tocilizumab | |
| symptomatic treatment only | 8 mg/kg intravenously over 1 | |
| (e.g., fever, nausea, fatigue, | hour (not to exceed 800 mg). | |
| headache, myalgia, malaise). | | |
| Grade 2 | Administer tocilizumab | Manage per Grade 3 |
| Symptoms require and | 8 mg/kg intravenously over 1 | if no improvement |
| respond to moderate | hour (not to exceed 800 mg). | within 24 hours after |
| intervention. | Repeat tocilizumab every 8 | starting tocilizumab. |
| Oxygen requirement less than | hours as needed if not | If improving, taper |
| 40% FiO2 or hypotension | responsive to intravenous fluids | corticosteroids. |
| responsive to fluids or low | or increasing supplemental | |
| dose of one vasopressor or | oxygen. Limit to a maximum of | |
| Grade 2 organ toxicity. | 3 doses in a 24-hour period; | |
| | maximum total of 4 doses if no | |
| | clinical improvement in the | |
| | signs and symptoms of CRS. | |
| | If improving, discontinue | |
| | tocilizumab. | |
| Grade 3 | Per Grade 2 | Administer |
| Symptoms require and | | methylprednisolone |
| respond to aggressive | | 1 mg/kg intravenously |
| intervention. | | twice daily or |
| Oxygen requirement greater | | equivalent |
| than or equal to 40% FiO2 or | | dexamethasone (e.g., |
| hypotension requiring high- | | 10 mg intravenously |
| dose or multiple vasopressors | | every 6 hours) until |
| or Grade 3 organ toxicity or | | Grade 1, then taper |
| Grade 4 transaminitis. | | corticosteroids. |
| | | If improving, manage |
| | | as Grade 2. |
| | | If not improving, |
| | | manage as Grade 4. |
| Grade 4 | Per Grade 2 | Administer |
| Life-threatening symptoms. | | methylprednisolone |
| Requirements for ventilator | | 1000 mg intravenously |
| support or continuous | | per day for 3 days. |
| veno-venous hemodialysis | | If improving, taper |
| (CVVHD), or Grade 4 organ | | corticosteroids, and |
| toxicity (excluding | | manage as Grade 3. |
| transaminitis). | | If not improving, |
| | | consider alternate |
| | | immunosuppressants. |

25. The method of any one of aspects 18 through 20 and 24, wherein neurologic toxicity in a B cell ALL subject is managed according to the following protocol:

| NE | | Revised Management |
|---|---|---|
| Grade | Original Management Guidelines | Guidelines |
| Grade 1 | Supportive care | Supportive care |
| | Neurological examination and | Closely monitor neurologic |
| | additional work-up as | status |
| | clinically indicated | Consider prophylactic |
| | | antiepileptic |
| Grade 2 | Supportive Care and Evaluation | Supportive Care and Evaluation |
| | Neurological examination, brain | Continuous cardiac telemetry |
| | MRI, and evaluation of CSF; | and pulse oximetry as |
| | consider EEG as clinically | indicated |
| | indicated | Serial neurological |
| | Consider prophylactic | examinations to include |
| | antiepileptic | fundoscopy and Glasgow |
| | | Coma Score, brain MRI, |
| | | evaluation of CSF, EEG; |
| | | consider neurology consult |
| | | Administer antiepileptics for |
| | | patients with seizures |
| | Tocilizumab | Tocilizumab |
| | Consider tocilizumab 8 mg/kg IV | For patients with |
| | over 1 hour (not to exceed 800 | concurrent CRS, administer |
| | mg) for patients with comorbid | tocilizumab 8 mg/kg IV over |
| | conditions (eg, grade ≥2 CRS) | 1 hour (not to exceed 800 mg); |
| | | repeat every 4-6 hours as |
| | | needed if not responsive to |
| | | IV fluids or increasing |
| | | supplemental oxygen, for a |
| | | maximum of 3 doses in 24 hours |
| | | Discontinue tocilizumab if |
| | | patient improves |
| | Corticosteroids | Corticosteroids |
| | N/A | For patients without |
| | | concurrent CRS, administer |
| | | dexamethasone 10 mg IV |
| | | every 6 hours |
| | | For patients with concurrent |
| | | CRS, if no improvement |
| | | within 24 hours after starting |
| | | tocilizumab, administer |
| | | dexamethasone 10 mg IV |
| | | every 6 hours |
| | | Taper corticosteroids if |
| | | patient improves |
| Grade 3 | Supportive Care and Evaluation | Supportive Care and Evaluation |
| | Per grade 2 | Manage in monitored care or |
| | Monitor with continuous cardiac | ICU |
| | telemetry and pulse oximetry | |
| | Tocilizumab | Tocilizumab |
| | Consider tocilizumab 8 mg/kg IV | Per grade 2 |
| | over 1 hour (not to exceed 800 | Discontinue tocilizumab if |
| | mg); repeat every 4-6 hours if | patient improves |
| | symptoms have not stabilized or | |
| | improved | |
| | Corticosteroids | Corticosteroids |
| | Consider corticosteroids (eg, | Administer dexamethasone |
| | dexamethasone 10 mg IV every | 10 mg IV every 6 hours |
| | 6 hours or methylprednisolone | Taper corticosteroids if |
| | 1 mg/kg BID) for worsening | patient improves |
| | symptoms despite tocilizumab | |
| Grade 4 | Supportive Care and Evaluation | Supportive Care and Evaluation |
| | Per grade 2 | Per grade 3 |
| | Monitor with continuous cardiac | Mechanical ventilation may |
| | telemetry and pulse oximetry | be required |
| | | Administer |
| | | immunosuppresants if patient |
| | | does not improve |
| | Tocilizumab | Tocilizumab |
| | Administer tocilizumab per grade | Per grade 2 |
| | 3 if not previously administered | |
| | Corticosteroids | Corticosteroids |
| | Administer corticosteroids (eg, | Administer high-dose |
| | methylprednisolone | corticosteroids (eg, |
| | 1 g/d × 3 days, followed by 250 | methylprednisone 1 g/d × 3 |
| | mg BID × 2 days, then 125 mg | days) |
| | BID × 2 days, then 60 mg | Taper corticosteroids if |
| | BID × 2 days) | patient improves |

26. The method of any one of aspects 1 through 25, wherein the B cell ALL subject may receive any one or more of the following bridging chemotherapy regimens:

Predefined Bridging Chemotherapy Regimens

| | |
|---|---|
| Attenuated | Vincristine non-liposomal (1-2 mg IV weekly) or |
| VAD | liposomal (2.25 mg/m² IV weekly), and |
| | dexamethasone 20-40 mg IV or PO daily × 3-4 |
| | days per week. Optional doxorubicin 50 mg/m² |
| | IV × 1 (first week only) |
| Mercaptopurine | 50-75 mg/m²/day by mouth (administer at bedtime |
| (6-MP) | on an empty stomach to improve absorption) |
| Hydroxyurea | Doses titrated between 15-50 mg/kg/day (rounded |
| | to the nearest 500 mg capsule and given as a single |
| | daily oral dose on a continuous basis) |
| DOMP | Dexamethasone 6 mg/m²/day PO (or IV) divided |
| | BID days 1-5, vincristine 1.5 mg/m² (maximum |
| | dose 2 mg) IV on day 1, methotrexate 20 mg/m² |
| | PO weekly, 6-MP 50-75 mg/m²/day PO daily |
| Attenuated | Fludarabine 30 mg/m² IV days 1-2, cytarabine |
| FLAG/ | 2 g/m² IV days 1-2, G-CSF 5 μg/kg SC or IV |
| FLAG-IDA | starts on day 3 and can continue until day before |
| | the start of conditioning chemotherapy. With or |
| | without idarubicin 6 mg/m² IV days 1-2 |
| Mini-hyper | Course A: Cyclophosphamide 150 mg/m² every |
| CVAD | 12 h × 3 days, dexamethasone 20 mg/d IV or PO |
| (courses A | daily days 1-4 and 11-14, vincristine 2 mg IV × 1 |

27. Autologous T cells expressing an anti-CD19 CAR for use in a method for treating mantle cell lymphoma (MCL) or B cell ALL according to any one of aspects 1 through 26.

28. Use of autologous T cells expressing an anti-CD19 CAR in the manufacture of a medicament for treating mantle cell lymphoma (MCL) or B cell ALL according to any one of aspects 1 through 26.

29. A method for treating mantle cell lymphoma (MCL) in a subject in need thereof comprising administering to the subject a therapeutically effective amount of a T cell product comprising autologous T cells expressing an anti-CD19 chimeric antigen receptor (CAR), wherein the MCL is relapsed or refractory MCL and the last prior therapy was less than 60 months prior to administration of the T cell product.

30. The method of aspect 29, wherein the MCL is refractory to, or has relapsed following, one or more of chemotherapy, radiotherapy, immunotherapy (including a T cell therapy and/or treatment with an antibody or antibody-drug conjugate), an autologous stem cell transplant, or any combination thereof.

31. The method of aspect 29 or 30, wherein the subject has received 1-3 prior treatments, wherein at least one of the prior treatments is selected from autologous SCT, anti-CD20 antibody, anthracycline- or bendamustine-containing chemotherapy, and/or a Bruton Tyrosine Kinase inhibitor (BTKi).

32. The method of aspect 31, wherein the BTKi is ibrutinib.

33. The method of aspect 32, wherein ibrutinib was the last treatment prior to administration of the T cell product.

34. The method of any one of aspects 29 through 33, wherein the subject has not received bridging therapy after leukapheresis and before conditioning/lymphodepleting chemotherapy.

35. The method of any one of aspects 29 through 34, wherein the subject has not received prior platinum therapy.

36. The method of any one of aspects 29 through 35, the subject receives a lymphodepleting chemotherapy regimen of cyclophosphamide 500 mg/m² intravenously and fludarabine 30 mg/m² intravenously, both given on each of the fifth, fourth, and third days before T cell infusion.

37. The method of any one of aspects 29 through 36, wherein the T cell product comprises CD4+ and CD8+ CAR T cells that are prepared from peripheral blood mononuclear cells (PBMCs) by positive enrichment and consequent partial or complete depletion of circulating cancer cells.

38. The method of aspect 37, wherein the PBMC are enriched for T cells by positive selection for CD4+ and CD8+ cells, activated with anti-CD3 and anti-CD28 antibodies in the presence of IL-2, and then transduced with a replication-incompetent viral vector containing FMC63-28Z CAR, a chimeric antigen receptor (CAR) comprising an anti-CD19 single-chain variable fragment (scFv), CD28 and CD3-zeta domains.

39. The method of aspects 37 or 38, wherein the T cell product comprises fewer cancer cells than a T cell product comprising T cells from a leukapheresis-derived product that have not been positively selected for CD4+ and CD8+ T cells.

40. The method of any one of aspects 37 to 39, wherein the T cell product has other superior product attributes relative to a T cell product comprising T cells from a leukapheresis-derived product that have not been positively selected/enriched for CD4+ and CD8+ T cells.

41. The method of aspect 40, wherein the superior product attributes are selected from increased percentage of CDRA45+CCR7+(naïve-like) T cells, decreased percentage of differentiated T cells, increased percentage of CD3+ cells, decreased IFN-gamma production, decreased percentage of CD3− cells.

42. The method of any one of aspects 29 through 41, wherein the subject is administered one or more doses of 1.8×10⁶, 1.9×10⁶, or 2×10⁶ CAR positive viable T cells per kg body weight, with a maximum of 2×10⁸ CAR positive viable T cells (for patients 100 kg and above).

43. The method of any one of aspects 29 through 42, wherein if the subject has achieved complete response to the first infusion, the subject may receive a second infusion of anti-CD19 CAR T cells, if progressing following >3 months of remission, provided CD19 expression has been retained and neutralizing antibodies against the CAR are not suspected, wherein response is assessed using the Lugano classification.

44. The method of any one of aspects 29 through 43, wherein the subject is monitored for signs and symptoms of cytokine release syndrome (CRS) and neurologic toxicity after T cell administration.

45. The method of aspect 44, wherein the subject is monitored daily for at least seven days, preferably for four weeks, following infusion for signs and symptoms of CRS and neurologic toxicity.

46. The method of any one of aspects 44 and 45, wherein the signs or symptoms associated with CRS comprise fever, chills, fatigue, tachycardia, nausea, hypoxia, and/or hypotension and the signs or symptoms associated with neurologic toxicity comprise encephalopathy, seizures, changes in level of consciousness, speech disorders, tremors, and/or confusion.

47. The method of any one of aspects 44 to 46, wherein cytokine release syndrome in MCL subjects is managed in accordance with the following protocol:

| CRS Grade | Tocilizumab | Corticosteroids |
|---|---|---|
| Grade 1 | If not improving after 24 | Not applicable. |
| Symptoms require | hours, administer tocilizumab | |
| symptomatic treatment only | 8 mg/kg intravenously over 1 | |
| (e.g., fever, nausea, fatigue, | hour (not to exceed 800 mg). | |
| headache, myalgia, malaise). | | |
| Grade 2 | Administer tocilizumab 8 | Manage per Grade 3 |
| Symptoms require and | mg/kg intravenously over 1 | if no improvement |
| respond to moderate | hour (not to exceed 800 mg). | within 24 hours after |
| intervention. | Repeat tocilizumab every 8 | starting tocilizumab. |
| Oxygen requirement less than | hours as needed if not | If improving, taper |
| 40% FiO2 or hypotension | responsive to intravenous fluids | corticosteroids. |
| responsive to fluids or low | or increasing supplemental | |
| dose of one vasopressor or | oxygen. Limit to a maximum of | |
| Grade 2 organ toxicity. | 3 doses in a 24-hour period; | |
| | maximum total of 4 doses if no | |
| | clinical improvement in the | |
| | signs and symptoms of CRS. | |
| | If improving, discontinue | |
| | tocilizumab. | |
| Grade 3 | Per Grade 2 | Administer |
| Symptoms require and | | methylprednisolone |
| respond to aggressive | | 1 mg/kg intravenously |
| intervention. | | twice daily or |
| Oxygen requirement greater | | equivalent |
| than or equal to 40% FiO2 or | | dexamethasone (e.g., |
| hypotension requiring high- | | 10 mg intravenously |
| dose or multiple vasopressors | | every 6 hours) until |
| or Grade 3 organ toxicity or | | Grade 1, then taper |
| Grade 4 transaminitis. | | corticosteroids. |
| | | If improving, manage |
| | | as Grade 2. |
| | | If not improving, |
| | | manage as Grade 4. |
| Grade 4 | Per Grade 2 | Administer |
| Life-threatening symptoms. | | methylprednisolone |
| Requirements for ventilator | | 1000 mg intravenously |
| support or continuous veno- | | per day for 3 days. |
| venous hemodialysis | | If improving, taper |
| (CVVHD), or Grade 4 organ | | corticosteroids, and |
| toxicity (excluding | | manage as Grade 3. |
| transaminitis). | | If not improving, |
| | | consider alternate |
| | | immunosuppressants. |

48. The method of any one of aspects 44 through 47, wherein neurologic toxicity in MCL subjects is managed in accordance with the following protocol:

| Grading | | |
|---|---|---|
| Assessment | Concurrent CRS | No Concurrent CRS |
| Grade 2 | Administer tocilizumab per claim | Administer dexamethasone 10 mg |
| | 15 for management of Grade 2 CRS. | intravenously every 6 hours until |
| | If not improving within 24 hours after | the event is Grade 1 or less, then |
| | starting tocilizumab, administer | taper corticosteroids. |
| | dexamethasone 10 mg intravenously | |
| | every 6 hours until the event is Grade 1 | |
| | or less, then taper corticosteroids. | |
| | If still not improving, manage as Grade 3. | |
| | Consider non-sedating anti-seizure medicines | |
| | (e.g., levetiracetam) for seizure prophylaxis. | |
| Grade 3 | Administer tocilizumab per claim | Administer dexamethasone 10 mg |
| | 15 for management of Grade 2 CRS. | intravenously every 6 hours. |
| | In addition, administer dexamethasone | Continue dexamethasone use |
| | 10 mg intravenously with the first dose of | until the event is Grade 1 or |
| | tocilizumab and repeat dexamethasone | less, then taper corticosteroids. |
| | dose every 6 hours. Continue | If not improving, manage as |
| | dexamethasone use until the event is | Grade 4. |
| | Grade 1 or less, then taper corticosteroids. | |
| | If improving, discontinue tocilizumab | |
| | and manage as Grade 2. | |
| | If still not improving, manage as | |
| | Grade 4 (below). | |
| | Consider non-sedating anti-seizure medicines | |
| | (e.g., levetiracetam) for seizure prophylaxis. | |
| Grade 4 | Administer tocilizumab per claim | Administer methylprednisolone |
| | 15 for management of Grade 2 CRS. | 1000 mg intravenously per day |
| | Administer methylprednisolone 1000 mg | for 3 days. |
| | intravenously per day with first dose of | If improving, then manage as |
| | tocilizumab and continue | Grade 3. |
| | methylprednisolone 1000 mg | If not improving, consider |
| | intravenously per day for 2 more days. | alternate immunosuppressants. |
| | If improving, then manage as Grade 3. | |
| | If not improving, consider | |
| | alternate immunosuppressants. | |

Consider non-sedating anti-seizure medicines
(e.g., levetiracetam) for seizure prophylaxis.

49. The method of any one of aspects 29 through 48, wherein the subject is a high-risk patient as determined by a Ki-67 tumor proliferation index ≥50% and/or presence of a TP53 mutation.

50. Autologous T cells expressing an anti-CD19 CAR for use in a method for treating MCL according to any one of aspects 29 through 49.

51, Use of autologous T cells expressing an anti-CD19 CAR in the manufacture of a medicament for treating MCL according to any one of aspects 29 through 50.

52. A method for treating a cancer selected from the group consisting of Waldenstrom Macroglobulinemia, Richter Transformation, Burkitt Lymphoma, and Hairy Cell Leukemia in a subject in need thereof comprising administering to the subject a therapeutically effective amount of a T cell product comprising autologous T cells expressing an anti-CD19 chimeric antigen receptor (CAR), wherein the subject receives bridging therapy after leukapheresis and before conditioning/lymphodepleting chemotherapy.

53. The method of aspect 52, wherein the cancer is refractory to, or has relapsed following, one or more of chemotherapy, radiotherapy, immunotherapy, an autologous stem cell transplant, or any combination thereof.

54. The method of aspect 52 or 53, wherein the bridging therapy is completed ≥7 days or 5 half-lives before a conditioning chemotherapy.

55. The method of any one of aspects 52 to 54, wherein the subject receives a lymphodepleting chemotherapy regimen of cyclophosphamide 500 mg/m² intravenously and fludarabine 30 mg/m² intravenously, both given on each of the fifth, fourth, and third days before T cell infusion.

56. The method of any one of aspects 52 to 55, wherein the cancer is Richter Transformation and the bridging therapy is selected from the group consisting of Rituximab, Cyclophosphamide, Hydroxydaunorubicin hydrochloride, vincristine, and Prednisone (R-CHOP): Dose Adjusted Etoposide, Prednisone, Vincristine, Cyclophosphamide, Doxorubicin, and Rituximab (DA-EPOCH-R); Bruton Tyrosine Kinase inhibitor (BTKi) (BTKi)±VTX-2337; dexamethasone; and irradiation.

57. The method of any one of aspects 52 to 55, wherein the cancer is Burkitt Lymphoma and the bridging therapy is selected from the group consisting of Rituximab, Ifosfamide, Carboplatin, and Etoposide (R-ICE); Dose Adjusted Etoposide, Prednisone, Vincristine, Cyclophosphamide, Doxorubicin, and Rituximab (DA-EPOCH-R); Rituximab, Gemcitabine, and Oxaliplatin (R-GEMOX); cyclophosphamide, vincristine sulfate, doxorubicin hydrochloride, and dexamethasone (HyperCVAD); dexamethasone; and irradiation.

58. The method of any one of aspects 52 to 55, wherein the cancer is Waldenstrom Macroglobulinemia and the bridging therapy is ibrutinib.

59. The method of any one of aspects 52 to 58, wherein the T cell product comprises CD4+ and CD8+ CAR T cells that are prepared from peripheral blood mononuclear cells (PBMCs) by positive enrichment and consequent partial or complete depletion of circulating cancer cells.

60. The method of aspect 59, wherein the PBMC are enriched for T cells by positive selection for CD4+ and CD8+ cells, activated with anti-CD3 and anti-CD28 antibodies in the presence of IL-2, and then transduced with a replication-incompetent viral vector containing FMC63-28Z CAR, a chimeric antigen receptor (CAR) comprising an anti-CD19 single-chain variable fragment (scFv), CD28 and CD3-zeta domains.

61. The method of aspects 59 or 60, wherein the T cell product comprises fewer cancer cells than a T cell product comprising T cells from a leukapheresis-derived product that has not been positively selected for CD4+ and CD8+ T cells.

62. The method of any one of aspects 59 to 61, wherein the T cell product has other superior product attributes relative to a T cell product comprising T cells from a leukapheresis-derived product that have not been positively selected/enriched for CD4+ and CD8+ T cells.

63. The method of aspect 62, wherein the superior product attributes are selected from increased percentage of CDRA45+CCR7+(naïve-like) T cells, decreased percentage of differentiated T cells, increased percentage of CD3+ cells, decreased IFN-gamma production, decreased percentage of CD3− cells.

64. The method of any one of aspects 52 through 63, wherein the subject is administered one or more doses of 1.8×10⁶, 1.9×10⁶, or 2×10⁶ CAR positive viable T cells per kg body weight, with a maximum of 2×10⁸ CAR positive viable T cells (for patients 100 kg and above).

65. The method of any one of aspects 52 through 64, wherein the subject is monitored for signs and symptoms of cytokine release syndrome (CRS) and neurologic toxicity after T cell administration.

66. The method of aspect 65, wherein the subject is monitored daily for at least seven days, preferably for four weeks, following infusion for signs and symptoms of CRS and neurologic toxicity.

67. The method of aspect 65 or 66, wherein the signs or symptoms associated with CRS include fever, chills, fatigue, tachycardia, nausea, hypoxia, and hypotension and the signs or symptoms associated with neurologic events include encephalopathy, seizures, changes in level of consciousness, speech disorders, tremors, and confusion.

68. Autologous T cells expressing an anti-CD19 CAR for use in a method for treating cancer according to any one of aspects 52 to 67.

69. Use of autologous T cells expressing an anti-CD19 CAR in the manufacturing of a medicament for treating cancer according to any one of aspects 52 through 67.

70. A method for treating a cancer in a subject in need thereof wherein the subject has previously been administered a first T cell product comprising autologous T cells expressing an anti-CD19 chimeric antigen receptor (CAR), further wherein a peripheral blood sample has been taken from the subject after administration of the first T cell product, comprising (a) measuring the level of CD8+CD27−CD28+ T-cells in the blood sample, and (b) if the level of CD8+CD27−CD28+ T-cells in the blood sample is elevated, administering to the subject a second T cell product.

71. A method for treating a cancer in a subject in need thereof wherein the subject has previously been administered a first T cell product comprising autologous T cells expressing an anti-CD19 chimeric antigen receptor (CAR), further wherein a peripheral blood sample has been taken from the subject after administration of the first T cell product, comprising (a) measuring the level of CD8+CCR7−CD45RA+CD27−CD28+ T-cells in the blood sample,

and (b) if the level of CD8+CCR7−CD45RA+CD27−CD28+ T-cells in the blood sample is elevated, administering to the subject a second T cell product.

72. The method of aspect 70 or 71, wherein the first T cell product comprises CD4+ and CD8+ T cells that have been prepared from peripheral blood mononuclear cells (PBMCs) by positive enrichment and consequent partial or complete depletion of circulating cancer cells.

73. The method of aspect 72, wherein the CD4+ and CD8+ T cells have been activated with anti-CD3 and anti-CD28 antibodies in the presence of IL-2, and then transduced with a replication-incompetent viral vector encoding, a chimeric antigen receptor (CAR) comprising an anti-CD19 single-chain variable fragment (scFv), CD28 and CD3-zeta domains.

74. The method of any one of aspects 70 to 73, wherein the cancer is selected from the group consisting of mantle cell lymphoma (MCL), B cell ALL, Waldenstrom Macroglobulinemia, Richter Transformation, Burkitt Lymphoma, and Hairy Cell Leukemia.

75. The method of aspect 74, wherein the cancer is MCL.

76. The method of any one of aspects 70 to 75, wherein the blood sample has been taken from the subject between day 5 and 9 after administration of the first T cell product.

77. The method of aspect 76, wherein the blood sample has been taken from the subject between day 6 and 8 after administration of the first T cell product.

78. The method of aspect 77, wherein the blood sample has been taken from the subject on Day 7 after administration of the first T cell product.

79. The method of any one of aspects 70 to 75, wherein the blood sample has been taken from the subject between day 12 and 16 after administration of the first T cell product.

80. The method of aspect 79, wherein the blood sample has been taken from the subject between day 13 and 15 after administration of the first T cell product.

81. The method of aspect 80, wherein the blood sample has been taken from the subject on Day 14 after administration of the first T cell product.

82. The method of aspect 70, wherein an elevated level of CD8+CD27−CD28+ T-cells for the subject is determined by comparison to other subjects who have received a comparable T cell product and have had a peripheral blood sample taken on the same day after T cell product administration.

83. The method of aspect 71, wherein an elevated level of CD8+CCR7−CD4SRA+CD27−CD28+ T-cells for the subject is determined by comparison to other subjects who have received a comparable T cell product and have had a peripheral blood sample taken on the same day after T cell product administration.

84. The method of any one of aspects 70 to 83, wherein the second T cell product is selected from the group consisting of an autologous CD19/CD20 bi-cistronic T-cell product and an allogenic T-cell product.

85. A T-cell product for use in a method for treating cancer according to any one of aspects 70 to 84.

86. Use of a T-cell product in the manufacture of a medicament for treating cancer according to any one of aspects 70 to 84.

87. A method for monitoring a subject who has previously been administered a first T cell product comprising autologous T cells expressing an anti-CD19 chimeric antigen receptor (CAR), comprising
(a) taking a blood sample from the subject after administration of the first T cell product,
(b) measuring the level of CD8+CD27−CD28+ T-cells in the blood sample, and
(c) prescribing a course of treatment based on the level of CD8+CD27−CD28+ T-cells in the blood sample, wherein if the level of CD8+CD27−CD28+ T-cells is elevated then a second T cell product is administered.

88. A method for monitoring a subject who has previously been administered a first T cell product comprising autologous T cells expressing an anti-CD19 chimeric antigen receptor (CAR), comprising
(a) taking a blood sample from the subject after administration of the first T cell product,
(b) measuring the level of CD8+CCR7−CD45RA+CD27−CD28+ T-cells in the blood sample, and
(c) prescribing a course of treatment based on the level of CD8+CCR7−CD45RA+CD27-CD28+ T-cells in the blood sample, wherein if the level of CD8+CCR7−CD45RA+CD27-CD28+ T-cells is elevated then a second T cell product is administered.

89. A method for monitoring a subject who has previously been administered a first T cell product comprising autologous T cells expressing an anti-CD19 chimeric antigen receptor (CAR), comprising
(a) taking a blood sample from the subject after administration of the first T cell product.
(b) measuring the level of CD27+CD28−CD4+CD3+ T-cells in the blood sample, and
(c) prescribing a course of treatment based on the level of CD27+CD28−CD4+CD3+ T-cells in the blood sample, wherein if the level of CD27+CD28−CD4+CD3+ T-cells is elevated then a second T cell product is not administered.

90. A method for monitoring a subject who has previously been administered a first T cell product comprising autologous T cells expressing an anti-CD19 chimeric antigen receptor (CAR), comprising
(a) taking a blood sample from the subject after administration of the first T cell product,
(b) measuring the level of PD1+CCR7+CD45RA− CD8+CD3+ T-cells in the blood sample, and
(c) prescribing a course of treatment based on the level of PD1+CCR7+CD45RA− CD8+CD3+ T-cells in the blood sample, wherein if the level of PD1+CCR7+CD45RA− CD8+CD3+ T-cells is elevated then a second T cell product is not administered.

### BRIEF DESCRIPTION OF THE DRAWINGS

FIG. 1 illustrates the ZUMA-2 study design. The superscripts are defined as follows: ^{a} Administered after leukapheresis and completed ≥5 days before initiating conditioning chemotherapy; PET-CT was required postbridging. ^{b} Bone marrow biopsy was to be done at screening and, if positive, not done, or indeterminate, a biopsy was needed to confirm CR. ^{c} After 3 months, only targeted AEs (neurological, hematological, infections, GVHD, autoimmune disorders, and secondary malignancies) were monitored and reported for 15 years after the initial anti-CD19 CAR T-cell infusion or until disease progression or initiation of subsequent anticancer therapy, whichever occurs first.

FIG. 2 illustrates patient response disposition at 24-month assessment. Complete response (CR). Partial response (PR).

### DETAILED DESCRIPTION

Except as otherwise expressly provided herein, each of the following terms shall have the meaning set forth below. Additional definitions are set forth throughout the application. Unless defined otherwise, all technical and scientific terms used herein have the meaning as commonly understood by one of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this application.

Units, prefixes, and symbols are denoted in their Système International de Unites (SI) accepted form. Numeric ranges are inclusive of the numbers defining the range. The disclosure provided herein are not limitations of the various aspects of the application, which may be by reference to the specification as a whole. Unless defined otherwise, all technical and scientific terms used herein have the same meaning as commonly understood by one of ordinary skill in the art to which this disclosure is related. For example, Juo, “The Concise Dictionary of Biomedicine and Molecular Biology”. 2nd ed., (2001). CRC Press; “The Dictionary of Cell & Molecular Biology”, 5th ed., (2013), Academic Press; and “The Oxford Dictionary Of Biochemistry And Molecular Biology”, Cammack et al. eds., 2nd ed. (2006), Oxford University Press, provide those of skill in the art with a general dictionary for many of the terms used in this disclosure.

The articles “a” or “an” refer to “one or more” of any recited or enumerated component.

The terms “about” or “comprising essentially of” refer to a value or composition that is within an acceptable error range for certain value or composition as determined by one of ordinary skill in the art, which will depend in part on how the value or composition is measured or determined, i.e., the limitations of the measurement system. For example, “about” or “comprising essentially of” may mean within 1 or more than 1 standard deviation per the practice in the art. Alternatively, “about” or “comprising essentially of” may mean a range of up to 10% (i.e., ±10%). For example, about 3 mg may include any number between 2.7 mg and 3.3 mg (for 10%). With respect to biological systems or processes, the terms may mean up to an order of magnitude or up to 5-fold of a value. When certain values or compositions are provided in the application and claims, unless otherwise stated, the meaning of “about” or “comprising essentially of” include an acceptable error range for that value or composition. Any concentration range, percentage range, ratio range, or integer range includes the value of any integer within the recited range and, when appropriate, fractions thereof (such as one-tenth and one-hundredth of an integer), unless otherwise indicated.

Unless specifically stated or obvious from context, as used herein, the term “or” is understood to be inclusive and covers both “or” and “and”. The term “and/or” refer to each of the two specified features or components with or without the other. Thus, the term “and/or” as used in a phrase such as “A and/or B” herein is intended to include “A and B.” “A or B,” “A” (alone), and “B” (alone). Similarly, the term “and/or” as used in a phrase such as “A, B, and/or C” is intended to encompass each of the following aspects: A, B. and C; A, B, or C; A or C; A or B; B or C: A and C: A and B; B and C; A (alone); B (alone); and C (alone).

The terms “e.g.,” and “i.e.” are used merely by way of example, without limitation intended, and not be construed as referring only those items explicitly enumerated in the specification.

The terms “or more”, “at least”. “more than”, and the like, e.g., “at least one” include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between. The term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”. “at least second”, and the like include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising.” or variations such as “comprises” or “comprising,” is understood to imply the inclusion of a stated element, integer or step, or group of elements, integers or steps, but not the exclusion of any other element, integer or step, or group of elements, integers or steps. It is understood that wherever aspects are described herein with the language “comprising.” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed invention.

Unless specifically stated or evident from context, as used herein, the term “about” refers to a value or composition that is within an acceptable error range for the particular value or composition as determined by one of ordinary skill in the art, which will depend in part on how the value or composition is measured or determined. i.e., the limitations of the measurement system. For example, “about” or “approximately” may mean within one or more than one standard deviation per the practice in the art. “About” or “approximately” may mean a range of up to 10% (i.e., +10%). Thus, “about” may be understood to be within 10%. 9%, 8%, 7%, 6%, 5%, 4%, 3%, 2%, 1%. 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

The term “activation,” “activated,” or the like refers to the state of a cell, including and not be limited to an immune cell (e.g., a T cell), that has been sufficiently stimulated to induce detectable cellular proliferation. Activation may be associated with induced cytokine production and detectable effector functions. The term “activated T cells” refers to, among other things, T cells that are undergoing cell division. T cell activation may be characterized by increased T cell expression of one or more biomarker, including, but not limited to, CD57. PD1, CD107a. CD25, CD137. CD69, and/or CD71. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. In general, such methods include contacting cells (such as T cells) with an activating, stimulatory, or costimulatory agent (such as anti-CD3 and/or anti-CD28 antibodies) which may be attached, coated, or bound to a bead or other surface, in a solution (such as feeding, culture, and/or growth medium) with certain cytokines (such as IL-2. IL-7, and/or IL-15). The activation agent (such as anti-CD3 and/or anti-CD28 antibodies) attached to the same bead serve as a “surrogate” antigen presenting cell (APC). One example is The Dynabeads® system, a CD3/CD28 activator/stimulator system for physiological activation of human T cells. In one embodiment, the T cells are activated and stimulated to proliferate with certain antibodies and/or cytokines using the methods described in U.S. Pat. Nos. 6,040,177 and 5,827,642 and PCT Publication No. WO 2012/129514, the contents of which are hereby incorporated by reference in their entirety.

The terms “administration,” “Administering” or the like refer to physical introduction of an agent to a subject, using any of the various methods and delivery systems known to those skilled in the art. Exemplary routes of administration for the immune cells prepared by the methods disclosed herein include intravenous (i.v. or IV), intramuscular, subcutaneous, intraperitoneal, spinal or other parenteral routes of administration, for example by injection or infusion. Parenteral route of administration refer to modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the immune cells (e.g., T cells) prepared by the present methods are administered via injection or infusion. Non-parenteral routes include a topical, epidermal or mucosal route of administration, for example, intranasally, vaginally, rectally, sublingually or topically. Administering may also be once, twice, or a plurality of times over one or more extended periods. Where one or more therapeutic agents (e.g., cells) are administered, the administration may be done concomitantly or sequentially. Sequential administration comprises administration of one agent only after administration of the other agent or agents has been completed.

The term “antibody” (Ab) includes, without limitation, an immunoglobulin which binds specifically to an antigen. In general, an antibody may comprise at least two heavy (H) chains and two light (L) chains interconnected by disulfide bonds. Each H chain comprises a heavy chain variable region (abbreviated herein as VH) and a heavy chain constant region. The heavy chain constant region may comprise three or four constant domains, CH1, CH2 CH3, and/or CH4. Each light chain comprises a light chain variable region (abbreviated herein as VL) and a light chain constant region. The light chain constant region may comprise one constant domain. CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, a monovalent and a divalent fragment or portion, and a single chain Ab.

An “antigen binding molecule,” “antibody fragment” or the like refer to any portion of an antibody less than the whole. An antigen binding molecule may include the antigenic complementarity determining regions (CDRs). Examples of antibody fragments include, but are not limited to. Fab, Fab′, F(ab′)2, and Fv fragments, dAb, linear antibodies, scFv antibodies, and multispecific antibodies formed from antigen binding molecules. In one aspect, the CD19 CAR construct comprises an anti-CD 19 single-chain FV. A “Single-chain Fv” or “scFv” antibody binding fragment comprises the variably heavy (V_{H}) and variable light (V_{L}) domains of an antibody, where these domains are present in a single polypeptide chain. Generally, the Fv polypeptide further comprises a polypeptide linker between the V_{H} and V_{L} domains, which enables the scFv to form the desired structure for antigen binding. All antibody-related terms used herein take the customary meaning in the art and are well understood by one of ordinary skill in the art.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, may serve as an antigen. An antigen may be endogenously expressed, i.e., expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

The term “neutralizing” refers to an antigen binding molecule, scFv, antibody, or a fragment thereof, that binds to a ligand and prevents or reduces the biological effect of that ligand. In some embodiments, the antigen binding molecule, scFv, antibody, or a fragment thereof, directly blocking a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “autologous” refers to any material derived from the same individual to which it is later to be re-introduced. For example, the engineered autologous cell therapy method described herein involves a collection of lymphocytes from an individual (such as a donor or a patient), which are then engineered to express a CAR construct and then administered back to the same individual.

The term “allogeneic” refers to any material derived from one individual which is then introduced to another individual of the same species, e.g., allogencic T cell transplantation.

The term “bridging therapy” refers to treatments given between apheresis/leukapheresis and the initiation of lymphodepleting/conditioning chemotherapy.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor at various stages. In one embodiment, the cancer or tumor is stage 0, such that, e.g., the cancer or tumor is very early in development and has not metastasized. In another embodiment, the cancer or tumor is stage I, such that, e.g., the cancer or tumor is relatively small in size, has not spread into nearby tissue, and has not metastasized. In other embodiment, the cancer or tumor is stage II or stage III, such that, e.g., the cancer or tumor is larger than in stage 0 or stage I. and it has grown into neighboring tissues but it has not metastasized, except potentially to the lymph nodes. In additional embodiment, the cancer or tumor is stage IV, such that, e.g., the cancer or tumor has metastasized. Stage IV may also be referred to as advanced or metastatic cancer.

An “anti-tumor effect” as used herein, refers to a biological effect that may present, and not being limited to, as a decrease in tumor volume, an inhibition of tumor growth, a decrease in the number of tumor cells, a decrease in tumor cell proliferation, a decrease in the number/extent of metastases, an increase in overall or progression-free survival, an increase in life expectancy, and/or amelioration of various physiological symptoms associated with the tumor. An anti-tumor effect may also refer to the prevention of the occurrence of a tumor, e.g., a vaccine.

The term “progression-free survival” (PFS) refers to the time from the treatment date to the date of disease progression (per general guidelines, such as revised IWG Response Criteria for Malignant Lymphoma) or death from any cause. The term “Disease progression” may be assessed by measurement of malignant lesions on radiographs or other methods should not be reported as adverse events. Death due to disease progression in the absence of signs and symptoms may be reported as the primary tumor type (e.g., DLBCL). The term “duration of response” (DOR) refers to the period of time between a subject's first objective response to the date of confirmed disease progression (per general guidelines, such as the revised IWG Response Criteria for Malignant Lymphoma) or death. The term “overall survival” (OS) refers to the time from the date of treatment to the date of death.

A “cytokine” refers to a non-antibody protein that may be released by immune cells, including macrophages, B cells, T cells, and mast cells to propagate an immune response. In one embodiment, one or more cytokines are released in response to the therapy. In other embodiment, those cytokines secreted in response to the therapy may indicate or suggest an effective therapy. In one embodiment, “cytokine” refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10. IL-12p40. IL-12p70. IL-15, and interferon (IFN) gamma. Examples of pro-inflammatory cytokines include, but are not limited to, IL-1a. IL-1b, IL-6, IL-13. IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, cotaxin, cotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

A “therapeutically effective amount,” “therapeutically effective dosage,” or the like refers to an amount of the cells (such as immune cells or engineered T cells) that are produced by the present methods (resulting in a T cell product) and that, when used alone or in combination with another therapeutic agent, protects or treats a subject against the onset of a disease or promotes disease regression as evidenced by a decrease in severity of disease symptoms, an increase in frequency and duration of disease symptom-free periods, and/or prevention of impairment or disability due to disease affliction. The ability to promote disease regression may be evaluated using a variety of methods known to the skilled practitioner, such as in subjects during clinical trials, in animal model systems predictive of efficacy in humans, or by assaying the activity of the agent in in vitro assays. In some embodiments, the donor T cells for use in the T cell therapy are obtained from the patient (e.g., for an autologous T cell therapy). In other embodiments, the donor T cells for use in the T cell therapy are obtained from a subject that is not the patient. The T cells may be administered at a therapeutically effective amount. For example, a therapeutically effective amount of the T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the CAR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the therapeutically effective amount of the CAR-positive viable T cells is between about 1×10⁶ and about 2×10⁶ CAR-positive viable T cells per kg body weight up to a maximum dose of about 1×10⁸ CAR-positive viable T cells. In some embodiments, the therapeutically effective amount of the CAR-positive viable T cells is between about 0.4×10⁸ and about 2×10⁸ CAR-positive viable T cells. In some embodiments, the therapeutically effective amount of the CAR-positive viable T cells is about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, about 1.5×10⁸, about 1.6×10⁸, about 1.7×10⁸, about 1.8×10⁸, about 1.9×10⁸, or about 2.0×10⁸ CAR-positive viable T cells.

The term “lymphocyte” as used herein may include natural killer (NK) cells, T cells, NK-T cells, or B cells. NK cells are a type of cytotoxic (cell toxic) lymphocyte that represent a major component of the inherent immune system. NK cells reject tumors and cells infected by viruses, through the process of apoptosis or programmed cell death. They were termed “natural killers” because they do not require activation to kill cells. T-cells play a major role in cell-mediated immunity (no antibody involvement). The T-cell receptors (TCR) differentiate themselves from other lymphocyte types. The thymus, a specialized organ of the immune system, is primarily responsible for the T cell's maturation.

There are several types of “immune cells,” including, without limitation, macrophages (e.g., tumor associated macrophages) neutrophils, basophils, cosinophils, granulocytes, natural killer cells (NK cells), B cells, T cells, NK-T cells, mast cells, tumor infiltrating lymphocytes (TILs), myeloid derived suppressor cells (MDSCs), and dendritic cells. The term also includes precursors of these immune cells. Hematopoietic stem and/or progenitor cells may be derived from bone marrow, umbilical cord blood, adult peripheral blood after cytokine mobilization, and the like, by methods known in the art. Some precursor cells are those that may differentiate into the lymphoid lineage, for example, hematopoietic stem cells or progenitor cells of the lymphoid lineage. Additional examples of immune cells that may be used for immune therapy are described in US Publication No. 20180273601, incorporated herein by reference in its entirety.

There are also several types of T-cells, namely: Helper T-cells (e.g., CD4+ cells, effector T_{EFF} cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+(L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and are CCR7⁺ and CD45RO⁺ and they secrete IL-2, but not IFNγ or IL-4, and (iii) effector memory T_{EM} cells, however, do not express L-selectin or CCR7 but do express CD45RO and produce effector cytokines like IFNγ and IL-4), Regulatory T-cells (Tregs, suppressor T cells, or CD4+CD25+ regulatory T cells), Natural Killer T-cells (NKT), and Gamma Delta T-cells. T cells found within tumors are referred to as “tumor infiltrating lymphocytes” (TIL). B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

A “naïve” T cell refers to a mature T cell that remains immunologically undifferentiated. Following positive and negative selection in the thymus. T cells emerge as either CD4⁺ or CD8⁺ naïve T cells. In their naïve state, T cells express L-selectin (CD62L⁺), IL-7 receptor-α (IL-7R-α), and CD132, but they do not express CD25, CD44, CD69, or CD45RO. As used herein, “immature” may also refer to a T cell which exhibits a phenotype characteristic of either a naïve T cell or an immature T cell, such as a T_{SCM} cell or a T_{CM} cell. For example, an immature T cell may express one or more of L-selectin (CD62L⁺), IL-7Rα, CD132, CCR7, CD45RA. CD45RO, CD27, CD28, CD95, IL-2Rβ, CXCR3, and LFA-1. Naïve or immature T cells may be contrasted with terminal differentiated effector T cells, such as T_{EM} cells and T_{EFF} cells.

“T cell function,” as referred to herein, refers to normal characteristics of healthy T cells. T cell function may comprise T cell proliferation, T cell activity, and/or cytolytic activity. In one embodiment, the methods of the present application of preparing T cells under certain oxygen and/or pressure condition would increase one or more T cell function, thereby making the T cells more fit and/or more potent for therapeutic purpose. In some embodiment. T cells that are prepared according to the present methods have increased T cell function as compared to those under conditions lacking certain oxygen and/or pressure. In other embodiment, T cells that are prepared according to the present methods would have increased T cell proliferation as compared to T cells cultured under conditions lacking certain oxygen and/or pressure. In additional embodiment. T cells that are prepared according to the present methods have increased T cell activity as compared to T cells cultured under conditions lacking certain oxygen and/or pressure. In further embodiment, T cells that are prepared according to the present methods have increased cytolytic activity as compared to T cells cultured under conditions lacking certain oxygen and/or pressure.

The terms cell “proliferation.” “proliferating” or the like refer to the ability of cells to grow in numbers through cell division. Proliferation may be measured by staining cells with carboxyfluorescein succinimidyl ester (CFSE). Cell proliferation may occur in vitro, e.g., during T cell culture, or in vivo. e.g., following administration of a immune cell therapy (e.g., T cell therapy). The cell proliferation may be measured or determined by the methods described herein or known in the field. For example, cell proliferation may be measured or determined by viable cell density (VCD) or total viable cell (TVC). VCD or TVC may be theoretical (an aliquot or sample is removed from a culture at certain timepoint to determine the cell number, then the cell number multiples with the culture volume at the beginning of the study) or actual (an aliquot or sample is removed from a culture at certain timepoint to determine the cell number, then the cell number multiples with the actual culture volume at the certain timepoint). The term “T cell activity” refers to any activity common to healthy T cells. In one embodiment, the T cell activity comprises cytokine production (such as INFγ, IL-2, and/or TNFα). In other embodiment, the T cell activity comprises production of one or more cytokine selected from interferon gamma (IFNγ or IFN-γ), tissue necrosis factor alpha (TNFα or IFNα), and both. The terms “cytolytic activity,” “cytotoxicity” or the like refer to the ability of a T cell to destroy a target cell. In one embodiment, the target cell is a cancer cell. e.g., a tumor cell. In other embodiment, the T cell expresses a chimeric antigen receptor (CAR) or a T cell receptor (TCR), and the target cell expresses a target antigen.

The term “genetically engineered.” “gene editing.” or “engineered” refers to a method of modifying the genome of a cell, including, but not being limited to, deleting a coding or non-coding region or a portion thereof or inserting a coding region or a portion thereof. In one embodiment, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Chimeric antigen receptors” (CARs or CAR-Ts) and the T cell receptors (TCRs) of the application are genetically engineered receptors. These engineered receptors may be readily inserted into and expressed by immune cells, including T cells, in accordance with techniques known in the art. With a CAR, a single receptor may be programmed to both recognize a specific antigen and, when bound to that antigen, activate the immune cell to attack and destroy the cell bearing or expressing that antigen. When these antigens exist on tumor cells, an immune cell that expresses the CAR may target and kill the tumor cell. In one embodiment, the cell that is prepared according to the present application is a cell having a chimeric antigen receptor (CAR), or a T cell receptor, comprising an antigen binding molecule, a costimulatory domain, and an activating domain. The costimulatory domain may comprise an extracellular domain, a transmembrane domain, and an intracellular domain. In one embodiment, the extracellular domain comprises a hinge or a truncated hinge domain.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The terms “immunotherapy” “immune therapy” or the like refer to the treatment of a subject afflicted with, or at risk of contracting or suffering a recurrence of, a disease by a method comprising inducing, enhancing, suppressing, or otherwise modifying an immune response. Examples of immunotherapy include, but are not limited to, T cell and NK cell therapies. T cell therapy may include adoptive T cell therapy, tumor-infiltrating lymphocyte (TIL) immunotherapy, autologous cell therapy, engineered autologous cell therapy and allogeneic T cell transplantation. One of skill in the art would recognize that the methods of preparing immune cells disclosed herein would enhance the effectiveness of any cancer or transplanted T cell therapy. Examples of T cell therapies are described in U.S. Patent Publication Nos. 2014/0154228 and 2002/0006409; U.S. Pat. Nos. 7,741,465; 6,319,494; and 5,728,388; and PCT Publication No. WO 2008/081035, which are incorporated by reference in their entirety.

The term “engineered Autologous Cell Therapy,” which may be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR) or T cell receptor (TCR). CAR positive (+) T cells are engineered to express an extracellular single chain variable fragment (scFv) with specificity for certain tumor antigen linked to an intracellular signaling part comprising a costimulatory domain and an activating domain. The costimulatory domain may be a signaling region derived from, e.g., CD28, CTLA4, CD16, OX-40, 4-1BB/CD137, CD2, CD7, CD27, CD30, CD40, programmed death-1 (PD-1), programmed death ligand-1 (PD-L1), inducible T cell costimulator (ICOS), ICOS-L, lymphocyte function-associated antigen-1 (LFA-1 (CD11a/CD18), CD3 gamma, CD3 delta, CD3 epsilon, CD247, CD276 (B7-H3), LIGHT (tumor necrosis factor superfamily member 14; TNFSF14), NKG2C, Ig alpha (CD79a), DAP-10, Fc gamma receptor, MHC class I molecule. TNF receptor proteins. Immunoglobulin-like proteins, cytokine receptors, integrins, signaling lymphocytic activation molecules (SLAM proteins), activating NK cell receptors, BTLA, a Toll ligand receptor, ICAM-1, B7-H3, CDS. ICAM-1. GITR, BAFFR, LIGHT, HVEM (LIGHTR), KIRDS2, SLAMF7, NKp80 (KLRF1), NKp44. NKp30, NKp46, CD19, CD4, CD8, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAMI (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The activating domain may be derived from, e.g., CD3, such as CD3 zeta, epsilon, delta, gamma, or the like. In one embodiment, the CAR is designed to have two, three, four, or more costimulatory domains. The CAR scFv may be designed to target, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in U.S. Patent Publication Nos. 2013/0287748, 2014/0227237, 2014/0099309, and 2014/0050708, which are hereby incorporated by reference in their entirety.

A “costimulatory signal.” as used herein, refers to a signal, which in combination with a primary signal, such as TCR/CD3 ligation, leads to a T cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18. CD19, CD19a, CD2, CD22, CD247, CD27. CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c. CD1-1d, CDS, CEACAM1, CRT AM, DAP-10, DNAMI (CD226), Fe gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D. NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

In some aspect, the cells of the present application may be obtained through T cells obtained from a subject. In one aspect, the T cells may be obtained from, e.g., peripheral blood mononuclear cells (PBMC), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. In some aspect, the cells collected by apheresis are washed to remove the plasma fraction and placed in an appropriate buffer or media for subsequent processing. In some aspect, the cells are washed with any solution (e.g., a solution with neutralized PH value or PBS) or culture medium. As will be appreciated, a washing step may be used, such as by using a semiautomated flow through centrifuge, e.g., the Cobe™ 2991 cell processor, the Baxter CytoMate™ M, or the like. In some aspect, the washed cells are resuspended in one or more biocompatible buffers, or other saline solution with or without buffer. In some aspect, the undesired components of the apheresis sample are removed. Additional methods of isolating T cells for a T cell therapy are disclosed in U.S. Patent Pub. No. 2013/0287748, which are hereby incorporated by references in their entirety.

In some embodiments, T cells are isolated from PBMCs by lysing the red blood cells and depleting the monocytes, e.g., by using centrifugation through a PERCOLL™ gradient. In some embodiments, a specific subpopulation of T cells, such as CD4+, CD8+, CD28+. CD45RA+, and CD45RO+ T cells is further isolated by positive or negative selection techniques known in the art. For example, enrichment of a T cell population by negative selection may be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected may be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In one embodiment, CD3+ T cells are isolated from PBMCs using Dynabeads coated with anti-CD3 antibody. CD8+ and CD4+ T cells are further separately isolated by positive selection using CD8 microbeads (e.g., Miltenyi Biotec) or CD4 microbeads (e.g., Miltenyi Biotec).

In some embodiments, PBMCs are used directly for genetic modification with the immune cells (such as CARs) using methods as described herein. In some embodiments, after isolating the PBMCs, T lymphocytes are further isolated, and both cytotoxic and helper T lymphocytes are sorted into naive, memory, and effector T cell subpopulations either before or after genetic modification and/or expansion.

The one or more immune cells described herein may be obtained from any source, including, for example, a human donor. The donor may be a subject in need of an anti-cancer treatment, e.g., treatment with immune cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor), immune cells may be differentiated in vitro from a hematopoietic stem cell population, or immune cells may be obtained from a donor. The population of immune cells may be obtained from the donor by any suitable method used in the art. For example, the population of lymphocytes may be obtained by any suitable extracorporeal method, venipuncture, or other blood collection method by which a sample of blood with or without lymphocytes is obtained. The population of lymphocytes is obtained by apheresis. The one or more immune cells may be collected from any tissue that comprises one or more immune cells, including, but not limited to, a tumor. A tumor or a portion thereof is collected from a subject, and one or more immune cells are isolated from the tumor tissue. Any T cell may be used in the methods disclosed herein, including any immune cells suitable for a T cell therapy. For example, the one or more cells useful for the application may be selected from the group consisting of tumor infiltrating lymphocytes (TIL), cytotoxic T cells, CAR T cells, engineered TCR T cells, natural killer T cells, Dendritic cells, and peripheral blood lymphocytes. T cells may be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. T cells may also be obtained from an artificial thymic organoid (ATO) cell culture system, which replicates the human thymic environment to support efficient ex vivo differentiation of T-cells from primary and reprogrammed pluripotent stem cells. Additional methods of isolating T cells for a T cell therapy are disclosed in U.S. Patent Publication No. 2013/0287748, in PCT Publication Nos. WO2015/120096 and WO2017/070395, all of which are herein incorporated by reference in their totality for the purposes of describing these methods and in their entirety. In one embodiment. T cells are tumor infiltrating leukocytes. In certain embodiment, the one or more T cells express CD8, e.g., are CD8⁺ T cells. In other embodiment, the one or more T cells express CD4. e.g., are CD4⁺ T cells. Additional methods of isolating T cells for a T cell therapy are disclosed in U.S. Patent Publication No. 2013/0287748, in PCT Publication Nos. WO2015/120096 and WO2017/070395, all of which are herein incorporated by reference in their totality for the purposes of describing these methods and in their entirety.

The immune cells and their precursor cells may be isolated by available methods (see, for example, Rowland-Jones et al., Lymphocytes: A Practical Approach, Oxford University Press, New York (1999)). The sources for the immune cells or precursor cells thereof include, but are not limited to, peripheral blood, umbilical cord blood, bone marrow, or other sources of hematopoietic cells. Negative selection methods may be used to remove cells that are not the desired immune cells. Additionally, positive selection methods may isolate or enrich for desired immune cells or precursor cells thereof, or a combination of positive and negative selection methods may be employed. Monoclonal antibodies (MAbs) are useful for identifying markers associated with certain cell lineages and/or stages of differentiation for both positive and negative selections. If certain type of cell is to be isolated, for example, certain type of T cell, various cell surface markers or combinations of markers, including but not limited to, CD3, CD4, CD8, CD34 (for hematopoietic stem and progenitor cells) and the like, may be used to separate the cells, as is well known in the art (see Kearse, T Cell Protocols: Development and Activation, Humana Press, Totowa N.J. (2000); De Libero, T Cell Protocols, Vol. 514 of Methods in Molecular Biology, Humana Press. Totowa N.J. (2009)).

PBMCs may be used directly for genetic modification with the immune cells (such as CARs). After isolating the PBMCs. T lymphocytes are further isolated, and both cytotoxic and helper T lymphocytes are sorted into naive, memory, and effector T cell subpopulations either before or after genetic modification and/or expansion. In one embodiment, CD8+ cells may be further sorted into naive, central memory, and effector cells by identifying cell surface antigens that are associated with each of these types of CD8+ cells. In other embodiment, the expression of phenotypic markers of central memory T cells includes CCR7, CD3, CD28, CD45RO, CD62L, and CD127 and are negative for granzyme B. In some embodiment, central memory T cells are CD8+, CD45RO+, and CD62L+ T cells. In certain embodiment, effector T cells are negative for CCR7, CD28, CD62L, and CD127 and positive for granzyme B and perforin. In additional embodiment, CD4+ T cells may be further sorted into subpopulations. For example, CD4+ T helper cells may be sorted into naive, central memory, and effector cells by identifying cell populations that have cell surface antigens.

The methods described herein further comprise enriching or preparing a population of immune cells obtained from a donor, between harvesting from the donor and exposing one or more cells obtained from a donor subject. Enrichment of a population of immune cells, e.g., the one or more T cells, may be accomplished by any suitable separation method including, but not limited to, the use of a separation medium (e.g., FICOLL-PAQUE™, ROSETTESEP™ HLA Total Lymphocyte enrichment cocktail, Lymphocyte Separation Medium (LSA) (MP Biomedical Cat. No. 0850494X), or the like), cell size, shape or density separation by filtration or elutriation, immunomagnetic separation (e.g., magnetic-activated cell sorting system, MACS), fluorescent separation (e.g., fluorescence activated cell sorting system, FACS), or bead-based column separation.

In one embodiment, the T cells are obtained from a donor subject. In other embodiment, the donor subject is human patient afflicted with a cancer or a tumor. In additional embodiment, the donor subject is a human patient not afflicted with a cancer or a tumor. The present application also provides a composition or formulation comprises a pharmaceutically acceptable carrier, diluent, solubilizer, emulsifier, preservative and/or adjuvant. In certain embodiment, the composition or formulation comprises an excipient. The terms composition and formulation are used interchangeably herein. The terms composition, a therapeutic composition, a therapeutically effective composition, pharmaceutical composition, pharmaceutically effective composition, and a pharmaceutically acceptable composition are used interchangeably herein. The composition may be selected for parenteral delivery, inhalation, or delivery through the digestive tract, such as orally. The composition may be prepared by known methods by one skilled person in the art. Buffers are used to maintain the composition at physiological pH or at a slightly lower pH, typically within a pH range of from about 5 to about 8. When parenteral administration is contemplated, the composition is in the form of a pyrogen-free, parenterally acceptable aqueous solution comprising a composition described herein, with or without additional therapeutic agents, in a pharmaceutically acceptable vehicle. By way of example, the vehicle for parenteral injection is sterile distilled water in which composition described herein, with or without at least one additional therapeutic agent, is formulated as a sterile, isotonic solution, properly preserved. The preparation involves the formulation of the desired agent with polymeric compounds (such as polylactic acid or polyglycolic acid), beads or liposomes, that provide for the controlled or sustained release of the product, which are then be delivered via a depot injection. In addition, implantable drug delivery devices may be used to introduce the desired therapeutic agent.

In some embodiments, the donor T cells for use in the T cell therapy are obtained from the patient (e.g., for an autologous T cell therapy). In other embodiments, the donor T cells for use in the T cell therapy are obtained from a subject that is not the patient. The T cells may be administered at a therapeutically effective amount. For example, a therapeutically effective amount of the T cells may be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T cells is about 10⁴ cells, about 10⁵ cells, about 10⁶ cells, about 10⁷ cells, or about 10⁸ cells. In some embodiments, the therapeutically effective amount of the CAR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the therapeutically effective amount of the CAR-positive viable T cells is between about 1×10⁶ and about 2×10⁶ CAR-positive viable T cells per kg body weight up to a maximum dose of about 1×10⁸ CAR-positive viable T cells.

A “patient” as used herein includes any human who is afflicted with a disease or disorder, including cancer (e.g., a lymphoma or a leukemia). The terms “subject” and “patient” are used interchangeably herein. The term “donor subject” refers to herein a subject whose cells are being obtained for further in vitro engineering. The donor subject may be a cancer patient that is to be treated with a population of cells generated by the methods described herein (i.e., an autologous donor), or may be an individual who donates a lymphocyte sample that, upon generation of the population of cells generated by the methods described herein, will be used to treat a different individual or cancer patient (i.e., an allogeneic donor). Those subjects who receive the cells that were prepared by the present methods may be referred to as “recipient subject.”

The terms “stimulation,” “stimulating,” or the like refer to a primary response induced by binding of a stimulatory molecule with its cognate ligand, wherein the binding mediates a signal transduction event. A “stimulatory molecule” is a molecule on a T cell. e.g., the T cell receptor (TCR)/CD3 complex, that specifically binds with a cognate stimulatory ligand present on an antigen present cell. A “stimulatory ligand” is a ligand that when present on an antigen presenting cell (e.g., an artificial antigen presenting cell (aAPC), a dendritic cell, a B-cell, and the like) may specifically bind with a stimulatory molecule on a T cell, thereby mediating a primary response by the T cell, including, but not limited to, activation, initiation of an immune response, proliferation, and the like. Stimulatory ligands include, but are not limited to, an MHC Class I molecule loaded with a peptide, an anti-CD3 antibody, a superagonist anti-CD28 antibody, and a superagonist anti-CD2 antibody. An “activated” or “active,” as used herein, refers to a T cell that has been stimulated. An active T cell may be characterized by expression of one or more marker selected form CD137, CD25, CD71, CD26, CD27, CD28, CD30, CD154, CD40L, and CD134.

The term “exogenous activation materials” refers to any activation substance derived from an external source. For example, exogenous anti-CD3 antibody, anti-CD28 antibody. IL-2, exogenous IL-7, or exogenous IL-15 may be obtained commercially or produced recombinantly. “Exogenous IL-2,” “Exogenous IL-7,” or “exogenous IL-15” when added in or contacted with one or more T cells, indicates that such IL-2, IL-7 and/or IL-15 are not produced by the T cells. The T cells prior to being mixed with “Exogenous” IL-2, IL-7 or IL-15 may contain a trace amount that were produced by the T cells or isolated from the subject with the T cells (i.e., endogenous “Exogenous” IL-2, IL-7 or IL-15). The one or more T cells described herein may be contacted with exogenous anti-CD3 antibody, anti-CD28 antibody. “Exogenous” IL-2, IL-7 and/or IL-15 through any means known in the art, including addition of isolated “Exogenous” IL-2, IL-7 and/or IL-15 to the culture, inclusion of anti-CD3 antibody, anti-CD28 antibody. “Exogenous” IL-2, IL-7 and/or IL-15 in the culture medium, or expression of “Exogenous” IL-2, IL-7 and/or IL-15 by one or more cells in the culture other than the one or more T cells, such as by a feeder layer.

As used herein, the term “in vitro cell” refers to any cell which is cultured ex vivo. In one embodiment, an in vitro cell includes a T cell.

The term “persistence” refers to the ability of, e.g., one or more transplanted immune cells administered to a subject or their progenies (e.g., differentiated or matured T cells) to remain in the subject at a detectable level for a period of time. As used herein, increasing the persistence of one or more transplanted immune cells or their progenies (e.g., differentiated or matured T cells) refers to increasing the amount of time the transplanted immune cells are detectable in a subject after administration. For example, the in vivo persistence of one or more transplanted immune cells may be increased by at least about at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 3 weeks, at least about 4 weeks, at least about 1 month, at least about 2 months, at least about 3 months, at least about 4 months, at least about 5 months, or at least about 6 months. In addition, the in vivo persistence of one or more transplanted immune cells may be increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold compared to the one or more transplanted immune cells that were not prepared by the present methods disclosed herein.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. The term “modulating” T cell maturation, as used herein, refers to the use of any intervention described herein to control the maturation and/or differentiation of one or more cells such as T cells. For example, modulating refers to inactivating, delaying or inhibiting T cell maturation. In another example, modulating refers to accelerating or promoting T cell maturation. The term “delaying or inhibiting T cell maturation” refers to maintaining one or more T cells in an immature or undifferentiated state. For example, “delaying or inhibiting T cell maturation” may refer to maintaining T cells in a naïve or T_{CM} state, as opposed to progressing to a T_{EM} or T_{EFF} state. In addition. “delaying or inhibiting T cell maturation” may refer to increasing or enriching the overall percentage of immature or undifferentiated T cells (e.g., naïve T cells and/or T_{CM} cells) within a mixed population of T cells. The state of a T cell (e.g., as mature or immature) may be determined, e.g., by screening for the expression of various genes and the presence of various proteins expressed on the surface of the T cells. For example, the presence of one or more marker selected from the group consisting of L-selectin (CD62L+), IL-7R-α, CD132, CR7, CD45RA, CD45RO, CD27, CD28, CD95, IL-2Rβ, CXCR3, LFA-1, and any combination thereof may be indicative of less mature, undifferentiated T cells.

“Treatment” or “treating” of a subject/patient refers to any type of intervention or process performed on, or the administration of one or more T cells prepared by the present application to, the subject/patient with the objective of reversing, alleviating, ameliorating, inhibiting, slowing down or preventing the onset, progression, development, severity or recurrence of a symptom, complication or condition, or biochemical indicia associated with a disease. In one aspect, “treatment” or “treating” includes a partial remission. In another aspect, “treatment” or “treating” includes a complete remission.

Various aspects of the application are described in further detail in the following subsections.

Patients with B-cell malignancies bearing high levels of circulating CD19-expressing tumor cells represent a population with very high unmet need. For example, Mantle Cell Lymphoma (MCL) is challenging to treat in the relapsed or refractory setting and remains incurable. No standard-of-care exists for second-line and higher chemotherapy. Treatment options include cytotoxic chemotherapy, proteasome inhibitors, immunomodulatory drugs, tyrosine kinase inhibitors, and stem cell transplant (both autologous [ASCT] and allogenic stem cell transplant [allo-SCT]). The choice of regimen is influenced by prior therapy, comorbidities and tumor chemosensitivity. Despite the high initial response rates observed with Bruton's tyrosine kinase inhibitor (BTK inhibitors), most patients will eventually develop progressive disease. New therapeutic strategies are needed to improve the dismal prognosis of patients with r/r MCL whose disease has not been effectively controlled with chemo-immunotherapy, stem cell transplant and the BTK inhibitors.

The anti-CD19 CAR T-cell therapy or product used in CD19 CAR-T may be manufactured from the patient's own T cells, via leukapheresis suitable for B-cell malignancies with circulating tumor cell burden to minimize the CD19-expressing tumor cells in the final product. The T cells from the harvested leukocytes from the leukapheresis product may be enriched by selection for CD4+/CD8+ T cells, activated with anti-CD3 and anti-CD28 antibodies, and/or transduced with a viral vector containing an anti-CD19 CAR gene. More details of the method may be found in PCT/US2015/014520 published as WO2015/120096 and in PCT/US2016/057983 published as WO2017/070395. In one embodiment, the cells are not treated with AKT inhibitors, IL-7, and IL-15. These engineered T cells may be propagated to generate a sufficient number of cells to achieve a therapeutic effect. Such process removes CD19-expressing malignant and normal B cells, which may reduce activation, expansion, and exhaustion of the anti-CD19 CAR T cells.

The activation, transduction, and/or expansion of immune cells may be conducted at any suitable time which allows for the production of (i) a sufficient number of cells in the population of engineered immune cells for at least one dose for administering to a patient, (ii) a population of engineered immune cells with a favorable proportion of juvenile cells compared to a typical longer process, or (iii) both (i) and (ii). The suitable time may factor several parameters, including the population of one or more cells, the cell surface receptor expressed by the immune cells, the vector used, the dose that is needed to have a therapeutic effect, and/or other variables. The time for activation may be 0 days, 1 day. 2 days, 3 days, 4 days, 5 days, 6 days, 7 days, 8 days, 9 days, 10 days, 11 days, 12 days, 13 days, 14 days, 15 days, 16 days, 17 days, 18 days, 19 days. 20 days, 21 days, or more than 21 days. The time for activation according to the method of present application would be reduced compared to expansion methods known in the art. For example, the time for activation may be shorter by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or may be shorter by more than 75%. Further, the time for expansion may be 0 days, 1 day. 2 days, 3 days, 4 days, 5 days, 6 days, 7 days, 8 days, 9 days, 10 days, 11 days, 12 days, 13 days, 14 days, 15 days, 16 days, 17 days, 18 days. 19 days, 20 days, 21 days, or more than 21 days. The time for expansion according to the method of present application would be reduced compared to expansion methods known in the art. For example, the time for expansion may be shorter by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or may be shorter by more than 75%. In one embodiment, the time for cell expansion is about 3 days, and the time from enrichment of the population of cells to producing the engineered immune cells is about 6 days.

The delay or inhibition of the maturation or differentiation of the one or more T cells or DC cells may be measured by any methods known in the art. For example, the delay or inhibition of the maturation or differentiation of the one or more T cells or DC cells may be measured by detecting the presence of one or biomarker. The presence of the one or more biomarker may be detected by any method known in the art, including, but not limited to, immunohistochemistry and/or fluorescence-activated cell sorting (FACS). The one or more biomarker is selected from the group consisting of L-selectin (CD62L⁺), IL-7Rα, CD132, CCR7, CD45RA, CD45RO. CD27, CD28, CD95, IL-2Rβ, CXCR3, LFA-1, or any combination thereof. In certain aspects, the delay or inhibition of the maturation or differentiation of the one or more T cells or DC cell) may be measured by detecting the presence of one or more of L-selectin (CD62L⁺), IL-7Rα, and CD132. One of skill in the art would recognize that though the present methods may increase the relative proportion of immature and undifferentiated T cells or DC cells in a population of collected cells, some mature and differentiated cells may still be present. As a result, the delay or inhibition of the maturation or differentiation of the one or more T cells or DC cells may be measured by calculating the total percent of immature and undifferentiated cells in a cell population before and after exposing one or more cells obtained from a donor subject to hypoxic culture conditions with or without pressures above atmospheric pressure. The methods disclosed herein may increase the percentage of immature and undifferentiated T cells in a T cell population.

The methods described herein further comprise stimulating the population of cells such as lymphocytes with one or more T-cell stimulating agents to produce a population of activated T cells under a suitable condition. Any combination of one or more suitable T-cell stimulating agents may be used to produce a population of activated T cells including, including, but not limited to, an antibody or functional fragment thereof which targets a T-cell stimulatory or co-stimulatory molecule (e.g., anti-CD2 antibody, anti-CD3 antibody (such as OKT-3), anti-CD28 antibody, or a functional fragment thereof), or any other suitable mitogen (e.g., tetradecanoyl phorbol acetate (TPA), phytohaemagglutinin (PHA), concanavalin A (conA), lipopolysaccharide (LPS), pokeweed mitogen (PWM)), or a natural ligand to a T-cell stimulatory or co-stimulatory molecule.

The suitable condition for stimulating or activating the population of immune cells as described herein further include a temperature, for an amount of time, and/or in the presence of a level of CO₂. The temperature for stimulation may be about 34° C., about 35° C. about 36° C., about 37° C., or about 38° C., about 34-38° C., about 35-37° C., about 36-38° C., about 36-37° C. or about 37° C.

Another condition for stimulating or activating the population of immune cells as described herein may further include a time for stimulation or activation. The time for stimulation is about 24-72 hours, about 24-36 hours, about 30-42 hours, about 36-48 hours, about 40-52 hours, about 42-54 hours, about 44-56 hours, about 46-58 hours, about 48-60 hours, about 54-66 hours, or about 60-72 hours, about 44-52 hours, about 40-44 hours, about 40-48 hours, about 40-52 hours, or about 40-56 hours. In one embodiment, the time for stimulation is about 48 hours or at least about 48 hours.

Other conditions for stimulating or activating the population of immune cells as described herein may further include a CO₂ Level. The level of CO₂ for stimulation is about 1.0-10% CO₂, about 1.0%, about 2.0%, about 3.0%, about 4.0%, about 5.0%, about 6.0%, about 7.0%, about 8.0%, about 9.0%, or about 10.0% CO₂, about 3-7% CO₂, about 4-6% CO₂, about 4.5-5.5% CO₂. In one embodiment, the level of CO₂ for stimulation is about 5% CO₂.

The conditions for stimulating or activating the population of immune cells may further comprise a temperature, for an amount of time for stimulation, and/or in the presence of a level of CO₂ in any combination. For example, the step of stimulating the population of immune cells may comprise stimulating the population of immune cells with one or more immune cell stimulating agents at a temperature of about 36-38° C., for an amount of time of about 44-52 hours, and in the presence of a level of CO₂ of about 4.5-5.5% CO₂. The one or more immune cells of the present application may be administered to a subject for use in immune or cell therapy. Accordingly, the one or more immune cells may be collected from a subject in need of a immune or cell therapy. Once collected, the one or more immune cells may be processed for any suitable period of time before being administered to a subject.

The concentration, amount, or population of lymphocytes or resulting product made by the methods herein is about 1.0-10.0×10⁶ cells/mL. In certain aspects, the concentration is about 1.0-2.0×10⁶ cells/mL, about 1.0-3.0×10⁶ cells/mL, about 1.0-4.0×10⁶ cells/mL, about 1.0-5.0×10⁶ cells/mL, about 1.0-6.0×10⁶ cells/mL, about 1.0-7.0×10⁶ cells/mL, about 1.0-8.0×10⁶ cells/mL. 1.0-9.0×10⁶ cells/mL, about 1.0-10.0×10⁶ cells/mL, about 1.0-1.2×10⁶ cells/mL, about 1.0-1.4×10⁶ cells/mL, about 1.0-1.6×10⁶ cells/mL, about 1.0-1.8×10⁶ cells/mL, about 1.0-2.0×10⁶ cells/mL, at least about 1.0×10⁶ cells/mL, at least about 1.1×10⁶ cells/mL, at least about 1.2×10⁶ cells/mL, at least about 1.3×10⁶ cells/mL, at least about 1.4×10⁶ cells/mL, at least about 1.5×10⁶ cells/mL, at least about 1.6×10⁶ cells/mL, at least about 1.7×10⁶ cells/mL, at least about 1.8×10⁶ cells/mL, at least about 1.9×10⁶ cells/mL, at least about 2.0×10⁶ cells/mL, at least about 4.0×10⁶ cells/mL, at least about 6.0×10⁶ cells/mL, at least about 8.0×10⁶ cells/mL, or at least about 10.0×10⁶ cells/mL.

An anti-CD3 antibody (or functional fragment thereof), an anti-CD28 antibody (or functional fragment thereof), or a combination of anti-CD3 and anti-CD28 antibodies may be used in accordance with the step of stimulating the population of lymphocytes, together or independently of exposing one or more cells obtained from a donor subject to hypoxic culture conditions with or without pressures above atmospheric pressure. Any soluble or immobilized anti-CD2, anti-CD3 and/or anti-CD28 antibody or functional fragment thereof may be used (e.g., clone OKT3 (anti-CD3), clone 145-2C11 (anti-CD3), clone UCHT1 (anti-CD3), clone L293 (anti-CD28), clone 15E8 (anti-CD28)). In some aspects, the antibodies may be purchased commercially from vendors known in the art including, but not limited to, Miltenyi Biotec, BD Biosciences (e.g., MACS GMP CD3 pure 1 mg/mL, Part No. 170-076-116), and eBioscience, Inc. Further, one skilled in the art would understand how to produce an anti-CD3 and/or anti-CD28 antibody by standard methods. In some aspect, the one or more T cell stimulating agents that are used in accordance with the step of stimulating the population of lymphocytes include an antibody or functional fragment thereof which targets a T-cell stimulatory or co-stimulatory molecule in the presence of a T cell cytokine. In one embodiment, the one or more T cell stimulating agents include an anti-CD3 antibody and IL-2. In certain embodiment, the T cell stimulating agent includes an anti-CD3 antibody at a concentration of 50 ng/mL. The concentration of anti-CD3 antibody is about 20 ng/mL-100 ng/mL, about 20 ng/mL, about 30 ng/mL, about 40 ng/mL, about 50 ng/ml, about 60 ng/mL, about 70 ng/ml, about 80 ng/ml, about 90 ng/mL, or about 100 ng/mL. In an alternative aspect, T cell activation is not needed.

The methods described herein further comprise transducing the population of activated immune cells with a viral vector comprising a nucleic acid molecule which encodes the cell surface receptor, using a single cycle or more of viral transduction to produce a population of transduced immune cells. Several recombinant viruses have been used as viral vectors to deliver genetic material to a cell. Viral vectors that may be used in accordance with the transduction step may be any ecotropic or amphotropic viral vector including, but not limited to, recombinant retroviral vectors, recombinant lentiviral vectors, recombinant adenoviral vectors, and recombinant adeno-associated viral (AAV) vectors. The method further comprises transducing the one or more immune cells with a retrovirus. In one aspect, the viral vector used to transduce the population of activated immune cells is an MSGV1 gamma retroviral vector. In one embodiment, the viral vector used to transduce the population of activated immune cells is the PG13-CD19-H3 Vector described by Kochenderfer, J. Immunother. 32(7): 689-702 (2009). According to one aspect of this aspect, the viral vector is grown in a suspension culture in a medium which is specific for viral vector manufacturing referred to herein as a viral vector inoculum. Any suitable growth media and/or supplements for growing viral vectors may be used in the viral vector inoculum in accordance with the methods described herein. According to some aspects, the viral vector inoculum is then added to the serum-free culture media described below during the transduction step. In some aspect, the one or more immune cells may be transduced with a retrovirus. In one embodiment, the retrovirus comprises a heterologous gene encoding a cell surface receptor. In another embodiment, the cell surface receptor may bind an antigen on the surface of a target cell, e.g., on the surface of a tumor cell. In addition to optionally exposing one or more cells obtained from a donor subject to hypoxic culture conditions with or without pressures above atmospheric pressure, the conditions for transducing the population of activated immune cells as described herein may comprise a specific time, at a specific temperature and/or in the presence of a specific level of CO₂. The temperature for transduction is about 34° C., about 35° C., about 36° C., about 37° C., or about 38° C., about 34-38° C., about 35-37° C., about 36-38° ° C., about 36-37° C. In one embodiment, the temperature for transduction is about 37° C. The predetermined temperature for transduction may be about 34° C., about 35° C. about 36° ° C., about 37° ° C., about 38° C., or about 39 ºC, about 34-39° C., about 35-37° C. In one embodiment, the predetermined temperature for transduction may be from about 36-38° C., about 36-37° C. or about 37° C. The time for transduction is about 12-36 hours, about 12-16 hours, about 12-20 hours, about 12-24 hours, about 12-28 hours, about 12-32 hours, about 20 hours or at least about 20 hours, is about 16-24 hours, about 14 hours, at least about 16 hours, at least about 18 hours, at least about 20 hours, at least about 22 hours, at least about 24 hours, or at least about 26 hours. The level of CO₂ for transduction is about 1.0-10% CO₂, about 1.0%, about 2.0%, about 3.0%, about 4.0%, about 5.0%, about 6.0%, about 7.0%, about 8.0%, about 9.0%, about 10.0% CO₂, about 3-7% CO₂, about 4-6% CO₂, about 4.5-5.5% CO₂, or about 5% CO₂.

Transducing the population of activated immune cells as described herein may be performed for a period of time, at certain temperature and/or in the presence of a specific level of CO₂ in any combination: a temperature of about 36-38° C., for an amount of time of about 16-24 hours, and in the presence of a level of CO₂ of about 4.5-5.5% CO: The immune cells may be prepared by the combination of any one of the methods of the application with any manufacturing method of preparing T cells for immunotherapy, including, without limitation, those described in PCT Publications Nos. WO2015/120096 and WO2017/070395, which are herein incorporated by reference in their totality for the purposes of describing these methods; any and all methods used in the preparation of Axicabtagene ciloleucel or Yescarta®; any and all methods used in the preparation of Tisagenlecleucel/Kymriah™; any and all methods used in the preparation of “off-the-shelf” T cells for immunotherapy; and any other methods of preparing lymphocytes for administration to humans. The manufacturing process may be adapted to remove circulating tumor cells from the cells obtained from the patient.

CAR-T cells may be engineered to express other molecules and may be of any one of the following exemplary types or others available in the art: first, second, third, fourth, fifth, or more CAR-T cells; Armored CAR-T cells, Motile CAR-T cells, TRUCK T-cells, Switch receptor CAR-T cells; Gene edited CAR T-cells; dual receptor CAR T-cells; suicide CAR T-cells, drug-inducible CAR-T cells, synNotch inducible CAR T-cells; and inhibitory CAR T-cells. In one aspect, the T cells are autologous T-cells. In one aspect, the T cells are autologous stem cells (for autologous stem cell therapy or ASCT). In one aspect, the T cells are non-autologous T-cells.

The cells (such as immune cells or T cells) are genetically modified following isolation or selection using known methods or activated and/or expanded (or differentiated in the case of progenitors) in vitro prior to being genetically modified. The immune cells, e.g., T cells, are genetically modified with the chimeric antigen receptors described herein (e.g., transduced with a viral vector comprising one or more nucleotide sequences encoding a CAR) and activated and/or expanded in vitro. Methods for activating and expanding T cells may be found in U.S. Pat. Nos. 6,905,874; 6,867,041; and 6,797,514; and PCT Publication No. WO 2012/079000, which are hereby incorporated by reference in their entirety. Generally, such methods may include contacting PBMC or isolated T cells with a stimulatory agent and costimulatory agent, such as anti-CD3 and/or anti-CD28 antibodies, that may be attached to a bead or other surface, in a culture medium with certain cytokines, such as IL-2. The Dynabeads® system, a CD3/CD28 activator/stimulator system for physiological activation of human T cells may be used. The T cells may be activated and stimulated to proliferate with suitable feeder cells, antibodies and/or cytokines as described in U.S. Pat. Nos. 6,040,177 and 5,827,642 and PCT Publication No. WO 2012/129514, which are hereby incorporated by reference in their entirety.

The cell surface receptor that is expressed by the engineered immune cells may be any antigen or molecule to be targeted by CAR, such as an anti-CD19 CAR, FMC63-28Z CAR, or FMC63-CD828BBZ CAR (Kochenderfer et al., J Immunother. 2009, 32(7): 689; Locke et al., Blood 2010, 116(20):4099, the subject matter of both of which is hereby incorporated by reference. In certain aspects, the predetermined dose of engineered immune cells may be more than about 1 million to less than about 3 million transduced engineered T cells/kg. In one embodiment, the predetermined dose of engineered T cells may be more than about 1 million to about 2 million transduced engineered T cells per kilogram of body weight (cells/kg). The predetermined dose of engineered T cells may be more than 1 million to about 2 million, at least about 2 million to less than about 3 million transduced engineered T cells per kilogram of body weight (cells/kg). In one embodiment, the predetermined dose of engineered T cells may be about 2 million transduced engineered T cells/kg. In another embodiment, the predetermined dose of engineered T cells may be at least about 2 million transduced engineered T cells/kg. Examples of the predetermined dose of engineered T cells may be about 2.0 million, about 2.1 million, about 2.2 million, about 2.3 million, about 2.4 million, about 2.5 million, about 2.6 million, about 2.7 million, about 2.8 million, or about 2.9 million transduced engineered T cells/kg.

The methods described herein comprise increasing or enriching the population of transduced one or more immune cells for a period of time to produce a population of engineered immune cells. The time for expansion may be any suitable time which allows for production of (i) a sufficient number of cells in the population of engineered immune cells for at least one dose for administering to a patient, (ii) a population of engineered immune cells with a favorable proportion of juvenile cells compared to a typical longer process, or (iii) both (i) and (ii). This time will depend on the cell surface receptor expressed by the immune cells, the vector used, the dose that is needed to have a therapeutic effect, and other variables. The predetermined time for expansion may be 0 days, 1 day, 2 days. 3 days, 4 days, 5 days, 6 days, 7 days, 8 days, 9 days. 10 days, 11 days, 12 days, 13 days, 14 days, 15 days, 16 days. 17 days, 18 days, 19 days, 20 days, 21 days, or more than 21 days. In one embodiment, the time for expansion of the present method is reduced compared to those known in the art. For example, the predetermined time for expansion may be shorter by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or may be shorter by more than 75%. In one example, the time for expansion is about 3 days, and the time from enrichment of the population of lymphocytes to producing the engineered immune cells is about 6 days.

The conditions for expanding the population of transduced immune cells may include a temperature and/or in the presence of a level of CO₂. In certain aspects, the temperature is about 34° C., about 35° C., about 36° C., about 37° ° C., or about 38° C., about 35-37° ° C., about 36-37° C., or about 37° ° C. The level of CO₂ is 1.0-10% CO₂, about 1.0%, about 2.0%, about 3.0%, about 4.0%, about 5.0%, about 6.0%, about 7.0%, about 8.0%, about 9.0%, about 10.0% CO₂, about 4.5-5.5% CO₂, about 5% CO₂, about 3.5%, about 4.0%, about 4.5%, about 5.0%, about 5.5%, or about 6.5% CO₂.

Each step of the methods described herein may be performed in a closed system. The closed system may be a closed bag culture system, using any suitable cell culture bags (e.g., Miltenyi Biotec MACS® GMP Cell Differentiation Bags, Origen Biomedical PermaLife Cell Culture bags). The cell culture bags used in the closed bag culture system may be coated with a recombinant human fibronectin fragment during the transduction step. The recombinant human fibronectin fragment may include three functional domains: a central cell-binding domain, heparin-binding domain II, and a CS1-sequence. The recombinant human fibronectin fragment may be used to increase gene efficiency of retroviral transduction of immune cells by aiding co-localization of target cells and viral vector. In one embodiment, the recombinant human fibronectin fragment is RETRONECTIN® (Takara Bio, Japan). The cell culture bags are coated with recombinant human fibronectin fragment at a concentration of about 1-60 μg/mL or about 1-40 μg/mL, about 1-20 μg/mL, 20-40 μg/mL, 40-60 μg/mL, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, about 20 μg/mL, about 2-5 μg/mL, about 2-10 μg/mL, about 2-20 μg/ml, about 2-25 μg/mL, about 2-30 μg/mL, about 2-35 μg/mL, about 2-40 μg/mL, about 2-50 μg/mL, about 2-60 μg/mL, at least about 2 μg/mL, at least about 5 μg/mL, at least about 10 μg/mL, at least about 15 μg/mL, at least about 20 μg/mL, at least about 25 μg/mL, at least about 30 μg/mL, at least about 40 μg/mL, at least about 50 μg/mL, or at least about 60 μg/mL recombinant human fibronectin fragment. In one embodiment, the cell culture bags are coated with at least about 10 μg/mL recombinant human fibronectin fragment. The cell culture bags used in the closed bag culture system may optionally be blocked with human albumin serum (HSA) during the transduction step. In another embodiment, the cell culture bags are not blocked with HSA during the transduction step.

The population of engineered immune cells produced by the methods described above may optionally be cryopreserved so that the cells may be used later. A method for cryopreservation of a population of engineered immune cells also is provided herein. Such a method may include a step of washing and concentrating the population of engineered immune cells with a diluent solution. For example, the diluent solution is normal saline, 0.9% saline, PlasmaLyte A (PL), 5% dextrose/0.45% NaCl saline solution (DS), human serum albumin (HSA), or a combination thereof. Also, HSA may be added to the washed and concentrated cells for improved cell viability and cell recovery after thawing. In another aspect, the washing solution is normal saline and washed and concentrated cells are supplemented with HSA (5%). The method may also include a step of generating a cryopreservation mixture, wherein the cryopreservation mixture includes the diluted population of cells in the diluent solution and a suitable cryopreservative solution. The cryopreservative solution may be any suitable cryopreservative solution including, but not limited to, CryoStor10 (BioLife Solution), mixed with the diluent solution of engineered immune cells at a ratio of 1:1 or 2:1. HSA may be added to provide a final concentration of about 1.0-10%, about 1.0%, about 2.0%, about 3.0%, about 4.0%, about 5.0%, about 6.0%, about 7.0%, about 8.0%, about 9.0%, about 10.0%, about 1-3% HSA, about 1-4% HSA, about 1-5% HSA, about 1-7% HSA, about 2-4% HSA, about 2-5% HSA, about 2-6% HSA, about 2-7% HAS or about 2.5% HSA in the cryopreserved mixture. Cryopreservation of a population of engineered immune cells may comprise washing cells with 0.9% normal saline, adding HSA at a final concentration of 5% to the washed cells, and diluting the cells 1:1 with CryoStor™ CS10 (for a final concentration of 2.5% HSA in the final cryopreservation mixture). In some aspect, the method also includes a step of freezing the cryopreservation mixture. Also, the cryopreservation mixture is frozen in a controlled rate freezer using a defined freeze cycle at a cell concentration of between about 1×10⁶ to about 1.5×10⁷ cells/mL of cryopreservation mixture. The method may also include a step of storing the cryopreservation mixture in vapor phase liquid nitrogen.

The population of engineered immune cells produced by the methods described herein may be cryopreserved at a predetermined dose. The predetermined dose may be a therapeutically effective dose, which may be any therapeutically effective dose as provided below. The predetermined dose of engineered immune cells may depend on the cell surface receptor that is expressed by the immune cells (e.g., the affinity and density of the cell surface receptors expressed on the cell), the type of target cell, the nature of the disease or pathological condition being treated, or a combination of both.

In one embodiment, the population of engineered T cells may be cryopreserved at a predetermined dose of about 1 million engineered T cells per kilogram of body weight (cells/kg). In certain embodiment, the population of engineered T cells may be cryopreserved at a predetermined dose of from about 500,000 to about 1 million engineered T cells/kg. In certain embodiment, the population of engineered T cells may be cryopreserved at a predetermined dose of at least about 1 million, at least about 2 million, at least about 3 million, at least about 4 million, at least about 5 million, at least about 6 million, at least about 7 million, at least about 8 million, at least about 9 million, at least about 10 million engineered T cells/kg. In other aspects, the population of engineered T cells may be cryopreserved at a predetermined dose of less than 1 million cells/kg, 1 million cells/kg, 2 million cells/kg. 3 million cells/kg. 4 million cells/kg, 5 million cells/kg, 6 million cells/kg, 7 million cells/kg, 8 million cells/kg. 9 million cells/kg, 10 million cells/kg, more than 10 million cells/kg, more than 20 million cells/kg, more than 30 million cells/kg, more than 40 million cells/kg, more than 50 million cells/kg, more than 60 million cells/kg, more than 70 million cells/kg, more than 80 million cells/kg, more than 90 million cells/kg, or more than 100 million cells/kg. In certain aspects, the population of engineered T cells may be cryopreserved at a predetermined dose of from about 1 million to about 2 million engineered T cells/kg. The population of engineered T cells may be cryopreserved at a predetermined dose between about 1 million cells to about 2 million cells/kg, about 1 million cells to about 3 million cells/kg, about 1 million cells to about 4 million cells/kg, about 1 million cells to about 5 million cells/kg, about 1 million cells to about 6 million cells/kg, about 1 million cells to about 7 million cells/kg, about 1 million cells to about 8 million cells/kg, about 1 million cells to about 9 million cells/kg, about 1 million cells to about 10 million cells/kg. The predetermined dose of the population of engineered T cells may be calculated based on a subject's body weight. In one example, the population of engineered T cells may be cryopreserved in about 0.5-200 mL of cryopreservation media. Additionally, the population of engineered T cells may be cryopreserved in about 0.5 mL, about 1.0 mL, about 5.0 mL, about 10.0 mL, about 20 mL, about 30 mL, about 40 mL, about 50 mL, about 60 mL, about 70 mL, about 80 mL, about 90 mL, or about 100 mL, about 10-30 mL, about 10-50 mL, about 10-70 mL, about 10-90 mL, about 50-70 mL, about 50-90 mL, about 50-110 mL, about 50-150 mL, or about 100-200 mL of cryopreservation media. In certain aspects, the population of engineered T cells may be preferably cryopreserved in about 50-70 mL of cryopreservation media.

In one embodiment, at least one of (a) contacting the population of immune cells with exogenous IL-2, exogenous IL-7, exogenous IL-15, and/or other cytokine(s), (b) stimulating the population of immune cells (c) transducing the population of activated immune cells, and (d) expanding the population of transduced immune cells is performed using a serum-free culture medium which is free from added serum. In some aspect, each of (a) to (d) is performed using a serum-free culture medium which is free from added serum. As referred to herein, the term “serum-free media” or “serum-free culture medium” means that the growth media used is not supplemented with serum (e.g., human serum or bovine serum). In other words, no serum is added to the culture medium as an individually separate and distinct ingredient for the purpose of supporting the viability, activation and grown of the cultured cells. Any suitable immune cell growth media may be used for culturing the cells in suspension in accordance with the methods described herein. For example, an immune cell growth media may include, but is not limited to, a sterile, low glucose solution that includes a suitable amount of buffer, magnesium, calcium, sodium pyruvate, and sodium bicarbonate. In one aspect, the T cell growth media is OPTMIZER™ (Life Technologies). In contrast to typical methods for producing engineered immune cells, the methods described herein may use culture medium that is not supplemented with serum (e.g., human or bovine).

The application provides various methods of treatment of cancer with T cells. In one aspect, the T cells are CAR-T cells against CD19, which may be prepared by the combination of any one of the methods of the application with any step of the manufacturing method of preparing T cells for immunotherapy, including, without limitation, those described in PCT Publication Nos. WO2015/120096 and WO2017/070395, both of which are herein incorporated by reference in their totality for the purposes of describing these methods; any and all methods used in the preparation of Axicabtagene ciloleucel or Yescarta®; any and all methods used in the preparation of Tisagenlecleucel/Kymriah™; any and all methods used in the preparation of “off-the-shelf” T cells for immunotherapy; and any other methods of preparing lymphocytes for administration to humans. In some aspect, the manufacturing process is adapted to specifically remove circulating tumor cells from the cells obtained from the patient.

In one aspect, the T cells are the CD19 CAR-T cells, prepared by the method described in PCT/US2016/057983. In one embodiment, a population of T cells that is depleted of circulating tumor cells is prepared from leukapheresis products. These cells may be prepared as described in PCT/US2016/057983 and are further described herein as CD19 CAR-T cells. Briefly, CD19 CAR-T is an autologous CAR T-cell product in which a subject's T cells are engineered to express receptors consisting of a single-chain antibody fragment against CD19 linked to CD28 and CD3ζ activating domains that result in elimination of CD 19-expressing cells. Following CAR engagement with CD19⁺ target cells, the CD3ζ domain activates the downstream signaling cascade that leads to T-cell activation, proliferation, and acquisition of effector functions, such as cytotoxicity. The intracellular signaling domain of CD28 provides a costimulatory signal that function with the primary CD3ζ signal to augment T-cell function, including interleukin (IL)-2 production. Together, these signals may stimulate proliferation of the CAR T cells and direct killing of target cells. In addition, activated T cells may secrete cytokines, chemokines, and other molecules that may recruit and activate additional antitumor immune cells. The anti-CD19 CAR in the CD19 CAR-T cells may comprise FMC63-28Z.

Due to the presence of circulating tumor cells in certain cancers, the manufacture of CD19 CAR-T includes a CD4⁺ and CD8⁺ T-cell enrichment step. The T-cell enrichment or isolation step may reduce circulating CD19-expressing tumor cells in leukapheresis material, and may relate to the activation, expansion, and exhaustion of the anti-CD19 CAR T cells during manufacturing.

The methods described herein may enhance the treatment outcome or effectiveness of an immune or cell therapy), which may be an adoptive T cell therapy selected from the group consisting of tumor-infiltrating lymphocyte (TIL) immunotherapy, autologous cell therapy, engineered autologous cell therapy (eACT™), allogeneic T cell transplantation, non-T cell transplantation, and any combination thereof. Adoptive T cell therapy broadly includes any method of selecting, enriching in vitro, and administering to a patient autologous or allogeneic T cells that recognize and are capable of binding tumor cells. TIL immunotherapy is a type of adoptive T cell therapy, wherein lymphocytes capable of infiltrating tumor tissue are isolated, enriched in vitro, and administered to a patient. The TIL cells may be either autologous or allogeneic. Autologous cell therapy is an adoptive T cell therapy that involves isolating T cells capable of targeting tumor cells from a patient, enriching the T cells in vitro, and administering the T cells back to the same patient. Allogeneic T cell transplantation may include transplant of naturally occurring T cells expanded ex vivo or genetically engineered T cells. Engineered autologous cell therapy, as described in more detail above, is an adoptive T cell therapy wherein a patient's own lymphocytes are isolated, genetically modified to express a tumor targeting molecule, expanded in vitro, and administered back to the patient. Non-T cell transplantation may include autologous or allogeneic therapies with non-T cells such as, but not limited to, natural killer (NK) cells.

The immune cell therapy of the present application is engineered Autologous Cell Therapy (eACT™). According to this aspect, the method may include collecting immune cells from a donor. The isolated immune cells may then be contacted with an exogenous activation reagent (e.g., cytokine), expanded, and engineered to express a chimeric antigen receptor (“engineered CAR T cells”) or T cell receptor (“engineered TCR T cells”). In some aspect, the engineered immune cells treat a tumor in the subject. For example, the one or more immune cells are transduced with a retrovirus comprising a heterologous gene encoding a cell surface receptor. In one embodimentx, the cell surface receptor is capable of binding an antigen on the surface of a target cell, e.g., on the surface of a tumor cell. In some embodiment, the cell surface receptor is a chimeric antigen receptor or a T cell receptor. In another embodiment, the one or more immune cells may be engineered to express a chimeric antigen receptor. The chimeric antigen receptor may comprise a binding molecule to a tumor antigen. The binding molecule may be an antibody or an antigen binding molecule thereof. For example, the antigen binding molecule may be selected from scFv, Fab. Fab′. Fv. F(ab′)2, and dAb, and any fragments or combinations thereof. The chimeric antigen receptor may further comprise a hinge region. The hinge region may be derived from the hinge region of IgG1, IgG2, IgG3, IgG4, IgA, IgD, IgE, IgM, CD28, or CD8 alpha. In one embodiment, the hinge region is derived from the hinge region of IgG4. The chimeric antigen receptor may also comprise a transmembrane domain. The transmembrane domain may be a transmembrane domain of any transmembrane molecule that is a co-receptor on immune cells or a transmembrane domain of a member of the immunoglobulin superfamily. In certain embodiment, the transmembrane domain is derived from a transmembrane domain of CD28, CD28T, CD8 alpha, CD4, or CD19. In another embodiment, the transmembrane domain comprises a domain derived from a CD28 transmembrane domain. In another embodiment, the transmembrane domain comprises a domain derived from a CD28T transmembrane domain. The chimeric antigen receptor may further comprise one or more costimulatory signaling regions. For example, the costimulatory signaling region may be a signaling region of CD28, CD28T, OX-40, 41BB, CD27, inducible T cell costimulator (ICOS), CD3 gamma, CD3 delta, CD3 epsilon, CD247. Ig alpha (CD79a), or Fe gamma receptor. In further embodiment, the costimulatory signaling region is a CD28 signaling region. In another embodiment, the costimulatory signaling region is a CD28T signaling region. In additional embodiment, the chimeric antigen receptor further comprises a CD3 zeta signaling domain.

In some aspects, the tumor antigen is selected from 707-AP (707 alanine proline), AFP (alpha (a)-fetoprotein). ART-4 (adenocarcinoma antigen recognized by T4 cells), BAGE (B antigen; b-catenin/m, b-catenin/mutated), BCMA (B cell maturation antigen), Ber-abl (breakpoint cluster region-Abelson), CAIX (carbonic anhydrase IX), CD19 (cluster of differentiation 19). CD20 (cluster of differentiation 20). CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated). CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor). G250 (glycoprotein 250). GAGE (G antigen), GD2 (disialoganglioside 2), GD3 (disialoganglioside 3), GnT-V (N-acetylglucosaminyltransferase V), Gp100 (glycoprotein 100KD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidase 2-a-Lfucosyltransferase), LeY (Lewis-Y antibody), L1CAM (L1 cell adhesion molecule), MAGE (melanoma antigen), MAGE-A1 (Melanoma-associated antigen 1), MAGE-A3, MAGE-A6, mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1, -2, -3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor ber-abl (protein of 190KD bcr-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2). SAGE (sarcoma antigen), SART-1 or SART-3 (squamous antigen rejecting tumor 1 or 3), SSX1, -2, -3, 4 (synovial sarcoma X1, -2, -3, -4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2). VEGF-R2 (vascular endothelial growth factor receptor 2), WT1 (Wilms' tumor gene), and any combination thereof. In one embodiment, the tumor antigen is CD19.

The T cell therapy comprises administering to the patient engineered T cells expressing T cell receptor (“engineered TCR T cells”). The T cell receptor (TCR) may comprise a binding molecule to a tumor antigen. In some aspects, the tumor antigen is selected from the group consisting of 707-AP, AFP, ART-4, BAGE, BCMA, Bcr-abl, CAIX, CD19, CD20, CD22, CD30, CD33, CD44v7/8, CAMEL, CAP-1. CASP-8, CDC27m, CDK4/m, CEA, CT. Cyp-B. DAM, EGFR, EGFRVIII, EGP-2, EGP-40, Erbb2, 3, 4, ELF2M, ETV6-AML1, FBP, fAchR. G250, GAGE, GD2, GD3, GnT-V. Gp100, HAGE, HER-2/neu, HLA-A, HPV. HSP70-2M, HST-2, hTERT or hTRT, ICE, IL-13R-a2, KIAA0205, KDR, x-light chain, LAGE, LDLR/FUT, LeY, L1CAM, MAGE, MAGE-A1, mesothelin, Murine CMV infected cells, MART-1/Melan-A, MC1R, Myosin/m, MUC1, MUM-1, -2, -3, NA88-A, NKG2D ligands, NY-BR-1, NY-ESO-1, oncofetal antigen. P15, p190 minor bcr-abl, Pml/RARa, PRAME, PSA, PSCA. PSMA. RAGE, RU1 or RU2, SAGE, SART-1 or SART-3, SSX1, -2, -3, 4, TAA, TAG-72, TEL/AML1, TPI/m, TRP-1. TRP-2, TRP-2/INT2, VEGF-R2, WT1, and any combination thereof.

“CD19-directed genetically modified autologous T cell immunotherapy” refers to a suspension of chimeric antigen receptor (CAR)-positive immune cells. An example of such immunotherapy is Clear CAR-T therapy, which uses CAR-T cells that are free of circulating tumor cells and enriched in CD4+/CD8+ T cells. Another example is axicabtagene ciloleucel (also known as Axi-cel™, YESCARTA™). See Kochenderfer, et al., (J Immunother 2009; 32:689 702). Other non-limiting examples include JCAR017, JCAR015, JCAR014, Kymriah (tisagenlecleucel), Uppsala U, anti-CD19 CAR (NCT02132624), and UCART19 (Celectis). See Sadelain et al. Nature Rev. Cancer Vol. 3 (2003), Ruella et al., Curr Hematol Malig Rep., Springer, NY (2016) and Sadelain et al. Cancer Discovery (April 2013) To prepare CD19-directed genetically modified autologous T cell immunotherapy, a patient's own T cells may be harvested and genetically modified ex vivo by retroviral transduction to express a chimeric antigen receptor (CAR) comprising a murine anti-CD19 single chain variable fragment (scFv) linked to CD28 and CD3-zeta co-stimulatory domains. In some embodiments, the CAR comprises a murine anti-CD19 single chain variable fragment (scFv) linked to 4-1BB and CD3-zeta co-stimulatory domain. The anti-CD19 CAR T cells may be expanded and infused back into the patient, where they may recognize and eliminate CD19-expressing target cells.

In one aspect, the TCR comprises a binding molecule to a viral oncogene. In one embodiment, the viral oncogene is selected from human papilloma virus (HPV), Epstein-Barr virus (EBV), and human T-lymphotropic virus (HTLV). In another embodiment, the TCR comprises a binding molecule to a testicular, placental, or fetal tumor antigen. In one embodiment, the testicular, placental, or fetal tumor antigen is selected from the group consisting of NY-ESO-1, synovial sarcoma X breakpoint 2 (SSX2), melanoma antigen (MAGE), and any combination thereof. In another embodiment, the TCR comprises a binding molecule to a lineage specific antigen. In additional embodiment, the lineage specific antigen is selected from the group consisting of melanoma antigen recognized by T cells 1 (MART-1), gp100, prostate specific antigen (PSA), prostate specific membrane antigen (PSMA), prostate stem cell antigen (PSCA), and any combination thereof. In certain embodiment, the T cell therapy comprises administering to the patient engineered CAR T cells expressing a chimeric antigen receptor that binds to CD19 and further comprises a CD28 costimulatory domain and a CD3-zeta signaling region. In additional embodiment, the T cell therapy comprises administering to a patient KTE-C19. In one aspect, the antigenic moieties also include, but are not limited to, an Epstein-Barr virus (EBV) antigen (e.g., EBNA-1, EBNA-2, EBNA-3, LMP-1, LMP-2), a hepatitis A virus antigen (e.g., VP1, VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gI, gJ, gK, gL, gM, UL20, UL32, US43, UL45, UL49A), a cytomegalovirus (CMV) viral antigen (e.g., glycoproteins gB, gC, gC, gE, gG, gH, gI, gJ, gK, gL, gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen. In such aspects, the cell surface receptor may be any TCR, or any CAR which recognizes any of the aforementioned viral antigens on a target virally infected cell. In other aspects, the antigenic moiety is associated with cells having an immune or inflammatory dysfunction. Such antigenic moieties may include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

The methods disclosed herein may involve a T cell therapy comprising the transfer of one or more T cells to a patient. The T cells may be administered at a therapeutically effective amount. For example, a therapeutically effective amount of T cells, e.g., engineered CAR+ T cells or engineered TCR+ T cells, may be at least about 10⁴ cells, at least about 10⁵ cells, at least about 10⁶ cells, at least about 10⁷ cells, at least about 10⁸ cells, at least about 10⁹, or at least about 10¹⁰ In another aspect, the therapeutically effective amount of the T cells, e.g., engineered CAR+ T cells or engineered TCR+ T cells, is about 10⁴ cells, about 10⁵ cells, about 10⁶ cells, about 10⁷ cells, or about 10⁸ cells. In one embodiment, the therapeutically effective amount of the T cells, e.g., engineered CAR+ T cells or engineered TCR+ T cells, is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In one embodiment, the amount of CD19 CAR-T cells is 2×10⁶ cells/kg, with a maximum dose of 2×10⁸ cells for subjects ≥100 kg. In another embodiment, the amount of CD19 CAR-T cells is 0.5×10⁶ cells/kg, with a maximum dose of 0.5×10⁸ cells for subjects ≥100 kg.

The patients may be preconditioned or lymphodepleted prior to administration of the T cell therapy. The patient may be preconditioned according to any methods known in the art, including, but not limited to, treatment with one or more chemotherapy drug and/or radiotherapy. In some aspects, the preconditioning may include any treatment that reduces the number of endogenous lymphocytes, removes a cytokine sink, increases a serum level of one or more homeostatic cytokines or pro-inflammatory factors, enhances an effector function of T cells administered after the conditioning, enhances antigen presenting cell activation and/or availability, or any combination thereof prior to a T cell therapy. The preconditioning may comprise increasing a serum level of one or more cytokines in the subject. The methods further comprise administering a chemotherapeutic. The chemotherapeutic may be a lymphodepleting (preconditioning) chemotherapeutic. Beneficial preconditioning treatment regimens, along with correlative beneficial biomarkers are described in U.S. Pat. No. 9,855,298, which is hereby incorporated by reference in its entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). One such dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient. In one aspect, the conditioning regimen comprises cyclophosphamide 500 mg/m²+fludarabine 30 mg/m² for 3 days. They may be administered at days −4, −3, and −2 or at days −5, −4, and −3 (day 0 being the day of administration of the cells). In one embodiment, the conditioning regimen comprises cyclophosphamide 200 mg/m², 250 mg/m², 300 mg/m², 400v. 500 mg/m² daily for 2, 3, or 4 days and fludarabine 20 mg/m². 25 mg/m², or 30 mg/m² for 2, 3, or 4 days. In one embodiment, and after leukapheresis, conditioning chemotherapy (fludarabine 30 mg/m²/day and cyclophosphamide 500 mg/m²/day) is administered on days−5,−4, and −3 prior to an intravenous infusion of a suspension of CD19 CAR-T cells. In some embodiments, the intravenous infusion time is between 15 and 120 minutes. In one embodiment, the intravenous infusion time is between 1 and 240 minutes. In some embodiments, the intravenous infusion time is up to 30 minutes. In some embodiments, the intravenous infusion time is up to 5, 10, 15, 20, 25, 30, 35, 40, 45, 50, 55, 60, 65, 70, 75, 80, 85, 90, 95, or up to 100 minutes. In some embodiments, the infusion volume is between 50 and 100 mL. In some embodiments, the infusion volume is between 20 and 100 ml. In some embodiments, the infusion volume is about 30, 35, 40, 45, 50, 55, 60, or about 65 ml. In some embodiments, the infusion volume is about 68 mL. In some embodiments, the suspension has been frozen and is used within 6, 5, 4, 3, 2, 1 hour of thawing. In some embodiments, the suspension has not been frozen. In some embodiments, the immunotherapy is infused from an infusion bag. In some embodiments, the infusion bag is agitated during the infusion. In some embodiments, the immunotherapy is administered within 3 hours after thawing. In some embodiments, the suspension further comprises albumin. In some embodiments, albumin is present in an amount of about 2-3% (v/v). In some embodiments, albumin is present in an amount of about 2.5% (v/v). In some embodiments, the albumin is present in an amount of about 1%, 2%, 3%, 4%, or 5% (v/v). In some embodiments, albumin is human albumin. In some embodiments, the suspension further comprises DMSO. In some embodiments, DMSO is present in an amount of about 4-6% (v/v). In some embodiments, DMSO is present in an amount of about 5% (v/v). In some embodiments, the DMSO is present in an amount of 1%. 2%, 3%, 4%, 5%, 6%, 7%, 8%, 9%, or 10% (v/v).

The methods disclosed herein may be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In certain aspects, the methods may induce a complete response. In other aspects, the methods may induce a partial response.

Cancers that may be treated include tumors that are not vascularized, not yet substantially vascularized, or vascularized. The cancer may also include solid or non-solid tumors.

In one embodiment, the method may be used to treat a B-cell malignancy bearing high levels of circulating CD19-expressing tumor cells and will be indicated for a distinct patient population with high unmet need.

In some embodiments, the CAR T cell intervention comprises T cells which are expanded from a T cell population depleted of circulating lymphoma cells and enriched for CD4+/CD8+ T cells by positive selection of mononuclear cells from a leukapheresis sample that is activated with anti-CD3 and anti-CD28 antibodies in the presence of IL-2, and then transduced with a replication-incompetent viral vector containing an anti-CD19 CAR construct. In some embodiments, the CAR construct is FMC63-28Z CAR. The CAR T cell generated using this method may be referred to as KTE-X19. In some embodiments, the cells are autologous. In some embodiments, the cells are heterologous. In some embodiments, the dose of CAR-positive T cells is 2×10⁶ anti-CD19 CAR T cells/kg. In some embodiments, the dose of CAR-positive T cells is 1×10⁶ anti-CD19 CAR T cells/kg. In some embodiments, the dose of CAR-positive T cells is 1.6×10⁶ anti-CD19 CAR T cells/kg, 1.8×10⁶ anti-CD19 CAR T cells/kg, or 1.9×10⁶ anti-CD19 CAR T cells/kg. In some embodiments, the CD19 CAR construct contains a CD3ζ T cell activation domain and CD28 signaling domain.

In some embodiments, the CAR T cells are administered as a single infusion on Day 0 following conditioning therapy with 25 mg/m²/day of fludarabine on Days −5, −4, and −3 and 900 mg/m²/day of cyclophosphamide on Day −2, after leukapheresis. In some embodiments, the conditioning therapy comprises 300 mg/m²/day of cyclophosphamide and 30 mg/m²/day of fludarabine for 3 days. In some embodiments, the conditioning chemotherapy comprises 30 mg/m²/day of fludarabine and 500 mg/m²/day of cyclophosphamide on Days −5, −4, and −3. In some embodiments, the patient may also have received acetaminophen and diphenhydramine or another H1-antihistamine approximately 30 to 60 minutes prior to infusion of anti-CD19 CAR T cells. In some embodiments, the patients receive one or more additional doses of anti-CD19 CAR T cells.

In some embodiments, the MCL cancer is relapsed/refractory MCL (r/r MCL). In some embodiments, the patient has received one or more prior treatments. In some embodiments, the patient has received 1-5 prior treatments. In some embodiments, the prior treatments may have included autologous SCT, anti-CD20 antibody, anthracycline- or bendamustine-containing chemotherapy, and/or a Bruton Tyrosine Kinase inhibitor (BTKi). In some embodiments, the BTKi is ibrutinib (Ibr). In some embodiments, the BTKi is acalabrutinib (Acala). In some embodiments, the disclosure provides that MCL patients who were previously treated with ibrutinib had a more pronounced response to anti-CD19 CAR T cell therapy as compared to patients previously treated with acalabrutinib. Accordingly, the disclosure provides a method of treating r/r MCL with anti-CD19 CAR T cell therapy wherein the patient has been previously treated with ibrutinib or acalabrutinib and whose cancer is, preferably, relapsed/refractory to the same. In some embodiments, the BTKi is tirabrutinib (ONO-4059), zanubrutinib (BGB-3111). CGI-1746 or spebrutinib (AVL-292, CC-292).

In some embodiments, the disclosure provides that for patients with prior Ibr, Acala, or both, median (range) peak CAR T cell levels were 95.9 (0.4-2589.5). 13.7 (0.2-182.4), or 115.9 (17.2-1753.6), respectively. In some embodiments, ORR/CR rates to anti-CD19 CAR T cell therapy in patients with MCL were 94%/65% in patients with prior Ibr. 80%/40% in patients with prior Acala, and 100%/100% in patients with both BTKis. In some embodiments, the 12-month survival rates in patients with prior Ibr, Acala, or both were 81%. 80%, or 100%, respectively. In some embodiments, CAR T cell expansion is associated with ORR/CR rate in patients previously treated with Ibr and/or Acala. Accordingly, in one embodiment, the patient is treated with both Ibr and Acala. In one embodiment, the disclosure provides a method of predicting ORR/CR in an MCL patient previously treated with Ibr and/or Acala by measuring peak CAR T cell levels and comparing them to a reference standard. In one embodiment, the disclosure provides a method of predicting ongoing response based on the measurement of CAR T cell peak levels/baseline tumor burden (CEN and INV). In one embodiment, the higher the ratio, the higher the likelihood of ongoing response at/by 12 months. In one embodiment, a ratio between 0.00001 and 0.005 is predictive of non-response at/by 12 months. In one embodiment, a ratio between 0.006 and 0.3 is predictive of relapse at/by 12 months. In one embodiment, a ration between 0.4 and 1 is predictive of ongoing response at/by 12 months. In one embodiment, the ratios may be determined by one of ordinary skill in the art from the average populations.

In some embodiments, the patient may have received bridging therapy (after leukapheresis and before chemotherapy) with dexamethasone (e.g., 20-40 mg or equivalent PO or IV daily for 1-4 days), methylprednisolone, ibrutinib (e.g., 560 mg PO daily), and/or acalabrutinib (e.g., 100 mg PO twice daily) after leukapheresis and completed, for example, in 5 days or less before conditioning chemotherapy. In some embodiments, such patient may have had high disease burden. In some embodiments, the bridging therapy is selected from an immunomodulator, R-CHOP, bendamustine, alkylating agents, and/or platinum-based agents.

In some embodiments, the disclosure provides that all MCL patients who responded to CAR T cell infusion achieved T cell expansion, whereas no expansion was observed in non-responding patients. In some embodiments, response is objective response (complete response+partial response). The disclosure provides that CAR T cell levels correlated with ORR in the first 28 days, where the area under the curve on days 0 to 28 (AUC₀₋₂₈) and peak levels were >200-fold higher in responders versus non-responders, suggesting that higher expansion led to better and perhaps deeper response as also indicated by the >80-fold higher peak/AUC CAR T cell levels in minimal residual disease (MRD, 10⁻⁵ sensitivity) negative compared with MRD positive patients (at week 4). Accordingly, the disclosure provides a method of predicting patient response and MRD to CAR T cell treatment of MCL comprising measuring peak/AUC CAR T cell levels and comparing them to a reference standard. In some embodiments, peak CAR T cell expansion is observed between Days 8 and 15 after CAR T cell administration. In some embodiments, CAR T cells levels are measured by qPCR. In some embodiments, the peak CAR T cell levels, AUC₀₋₂₈, and/or MRD are monitored by next-generation sequencing. In some examples, the CAR T cell numbers are measured in cells/microliter of blood. In some examples, the CAR T cell numbers are measured by the number of CAR gene copies/μg of host DNA. In some examples, the CAR T cell numbers are measured as described in Kochenderfer J. N et al. J. Clin. Oncol. 2015; 33:540-549. In one embodiment, CAR T cell levels are measured as described in Locke F L et al. Mol Ther. 2017; 25(1):285-295.

In some embodiments, the disclosure provides that CAR T cell expansion was greater in MCL patients with grade ≥3 than in those with grade≤3 CRS and NE events. Accordingly, the disclosure provides a method of predicting grade ≥3 CRS and NE events comprising measuring CAR T cell expansion after CAR T cell treatment and comparing the levels to a reference value, wherein the higher the CAR T cell expansion, the higher the chance for grade ≥3 CRS and NE events.

In some embodiments, the cytokine levels are measured by and are protein or mRNA levels (which ones). In some embodiments, the cytokine levels are measured as described in Locke F L et al. Mol Ther. 2017; 25(1):285-295.

In some embodiments, the disclosure provides that serum GM-CSF and IL-6 peak levels (reached about 8 days post CAR T cell administration) were positively associated with grade ≥3 CRS and grade ≥3 NE in MCL patients. Accordingly, the disclosure provides a method of predicting grade ≥3 CRS and grade ≥3 NE comprising measuring the peak levels of GM-CSF and IL-6 post-CAR T cell administration and comparing them to a reference level, wherein the higher the peak level of these cytokines, the higher the chance for grade ≥3 CRS and NE.

In some embodiments, the disclosure provides that serum ferritin was positively associated with grade ≥3 CRS in MCL patients. Accordingly, the disclosure provides a method of predicting grade ≥3 CRS comprising measuring the peak levels of serum ferritin post-CAR T cell administration and comparing them to a reference level, wherein the higher the peak level of ferritin, the higher the chance for grade ≥3 CRS.

In some embodiments, the disclosure provides that serum IL-2 and IFN-gamma were positively associated with grade ≥3 NE in MCL patients. Accordingly, the disclosure provides a method of predicting grade ≥3 CRS comprising measuring the peak levels of serum IL-2 and IFN-gamma post-CAR T cell administration and comparing them to a reference level, wherein the higher the peak level of IL-2 and IFN-gamma, the higher the chance for grade ≥3 NE.

In some embodiments, the disclosure provides that cerebrospinal fluid levels of C-reactive protein, ferritin, IL-6, IL-8, and vascular cell adhesion molecule (VCAM) were positively associated with grade ≥3 NE in MCL patients. Accordingly, the disclosure provides a method of predicting grade ≥3 CRS comprising measuring the cerebrospinal fluid levels of C-reactive protein, ferritin, IL-6, IL-8, and/or vascular cell adhesion molecule (VCAM) post-CAR T cell administration and comparing them to a reference level, wherein the higher the cerebrospinal fluid levels of C-reactive protein, ferritin, IL-6, IL-8, and/or vascular cell adhesion molecule (VCAM), the higher the chance for grade ≥3 NE.

In some embodiments, the disclosure provides that peak serum levels of cytokines associated positively with Grade ≥3 CRS included IL-15, IL-2Rα, IL-6, TNFα, GM-CSF, ferritin, IL-10, IL-8, MIP-1a, MIP-1b, granzyme A, granzyme B, and perforin. In some embodiments, the disclosure provides that peak serum levels of cytokines associated with Grade ≥3 NE included IL-2, IL-1 Ra, IL-6, TNFα, GM-CSF, IL-12p40, IFN-γ, IL-10, MCP-4, MIP-1b, and granzyme B. In some embodiments, the disclosure provides that cytokines associated with both Grade ≥3 CRS and NE included IL-6, TNFα, GM-CSF, IL-10, MIP-1b, and granzyme B. In some embodiments, cytokine serum levels peak within 7 days of CAR T cell administration. Accordingly, the disclosure provides a method of predicting grade ≥3 CRS post-CAR T cell administration comprising measuring peak serum levels of IL-15, IL-2Rα, IL-6, TNFα, GM-CSF, ferritin, IL-10, IL-8, MIP-1a, MIP-1b, granzyme A, granzyme B, and/or perforin after anti-CD19 CAR T treatment and comparing the levels to a reference standard. Accordingly, the disclosure also provides a method of predicting grade ≥3 CRS and grade ≥3 NE in MCL comprising measuring peak serum levels of IL-6, TNFα, GM-CSF, IL-10, MIP-1b, and granzyme B after anti-CD19 CAR T treatment and comparing the levels to a reference standard.

In some embodiments, the disclosure provides that there was a trend for increased proliferative (IL-15, IL-2) and inflammatory (IL-6, IL-2Rα, sPD-L1 and VCAM-1) peak cytokine levels in patients with MCL with mutated TP53 vs wild-type TP53. Accordingly, in some embodiments, the disclosure provides a method of improving response to CAR T cell treatment in MCL comprising manipulating the levels of proliferative and/or inflammatory cytokines after CAR T cell administration.

In some embodiments, the disclosure provides that for patients that were MRD negative at one month post CAR T cell administration, there was an increase in peak levels of IFN-gamma and IL-6, and a trend towards increased IL-2, relative to patients that were MRD positive at one month. Accordingly, the disclosure provides a method of predicting whether a patient is MRD negative in MCL comprising measuring peak serum levels of IFN-gamma, IL-6, and/or IL-2 after anti-CD19 CAR T treatment and comparing the level to a reference standard.

In some embodiments, the disclosure is directed to a T cells product whereby the T cells are expanded from a T cell population depleted of circulating lymphoma cells and enriched for CD4+/CD8+ T cells by positive selection of mononuclear cells from a leukapheresis sample that is activated with anti-CD3 and anti-CD28 antibodies in the presence of IL-2, and then transduced with a replication-incompetent viral vector containing an anti-CD19 CAR construct. In some embodiments, such T cell product may be used to treat ALL, CLL. AML. In some embodiments, the CAR construct is FMC63-28Z CAR. In some embodiments, the cells are autologous. In some embodiments, the cells are heterologous. In some embodiments, the dose of CAR-positive T cells is 2×10⁶ anti-CD19 CAR T cells/kg. In some embodiments, the dose of CAR-positive T cells is 1×10⁶ anti-CD 19 CAR T cells/kg. In some embodiments, the dose of CAR-positive T cells is 1.6×10⁶ anti-CD19 CAR T cells/kg. 1.8×10⁶ anti-CD19 CAR T cells/kg, or 1.9×10⁶ anti-CD19 CAR T cells/kg. In some embodiments, the CD19 CAR construct contains a CD3ζ T cell activation domain and CD28 signaling domain. In some embodiments, the T cell product is KTE-X19. In some embodiments, the disclosure provides that the anti-CAR T cell product prepared as described in the preceding paragraph may be used in B cell ALL and B cell NHL. In some embodiments, the product characteristics may be selected from percentage of T cells of specific subsets (naïve, central memory, effector, and effector memory), percentage of CD4+ cells, percentage of CD8+ cells and CD4/CD8 ratio. In some embodiments, the product characteristic is the level of IFNγ production in co-culture (pg/mL) with target CD19-expressing cancer cells (e.g., Toledo) cells mixed in a 1:1 ratio with the anti-CD19 CAR T product cells. In one embodiment, IFNγ may be measured in cell culture media 24 h post-incubation using a qualified ELISA. In some embodiments, one or more of these product characteristics is superior to those of anti-CAR T cells prepared from leukapheresis without CD4+/CD8+ positive cell enrichment. In some embodiments, the superior product characteristic may be selected from increased percentage of cells with naïve phenotype (CD45RA+CCR7+), decreased percentage of cells with differentiated phenotype (CCR7−), decreased level of IFNγ-producing cells, and increased level of CD8+ cells. In some embodiments, the anti-CD19 T cell product comprises T_{CM}, central memory T cells (CD45RA-CCR7+); T_{EFF}, effector T cells (CD45RA+CCR7−); T_{EM}, effector memory T cells (CD45RA-CCR7−); and/or T_{N}, naïve-like T cells (CD45RA+CCR7+). In some embodiments, the product comprises T_{N} naïve-like T cells means T cells that are CD45RA+CCR7+ and comprises stem-like memory cells. In some embodiments, the T cell product is KTE-X19. In some embodiments, KTE-X19 has ≥190 μg/mL. IFN-γ production. In certain embodiment, KTE-X19 has ≥90% of CD3+ cells. In some other embodiments, the percentage of NK cells in KTE-X19 is 0.1% (range 0.0%-2.8%). In some additional embodiments, the percentage of CD3 cellular impurities in KTE-X19 is 0.5% (range 0.3%-3.9%).

In some embodiments, the cancer is relapsed/refractory B cell ALL. In some embodiments, the patient is ≤21 years old. In some embodiments, the patient is ≤21 years old, weighs ≥10 kg, and has B cell ALL that is primary refractory, relapsed within 18 months of first diagnosis, R/R after ≥2 lines of systemic therapy, or R/R after allogeneic stem cell transplantation at least 100 days prior to enrollment. In one embodiment, the cancer is indolent lymphoma or leukemia. In one embodiment, the cancer is an aggressive B-cell lymphoma, which include many types, subtypes and variants of diffuse large B-cell lymphoma (DLBCL), Burkitt lymphoma (BL), mantle cell lymphoma and its blastoid variant, and B lymphoblastic lymphoma. DLBCL may be DLBCL NOS. T-cell/histiocyte-rich large B-cell lymphoma, Primary DLBCL of the CNS, Primary cutaneous DLBCL, leg type. EBV-positive DLBCL of the elderly. Other lymphomas of large B cells include Primary mediastinal (thymic) LBCL, DLBCL associated with chronic inflammation, Lymphomatoid granulomatosis. ALK-positive LBCL, Plasmablastic lymphoma, Large B-cell lymphoma arising in HHV8-associated multicentric Castleman disease, and Primary effusion lymphoma. Other types of lymphomas include B-cell lymphoma, unclassifiable, with features intermediate between DLBCL, and Burkitt's lymphoma and B-cell lymphoma, unclassifiable, with features intermediate between DLBCL and classical Hodgkin lymphoma, Splenic marginal zone B-cell lymphoma, Extranodal marginal zone B-cell lymphoma of MALT. Nodal marginal zone B-cell lymphoma, Hairy cell leukemia. Lymphoplasmacytic lymphoma (Waldenstrom macroglobulinemia), Richter transformation, and Primary effusion lymphoma. The cancer may be at any stage, from stage 1 through stage 4.

In some embodiments, the conditioning chemotherapy/lymphodepleting regimen is administered after ≥7 days or 5 half-lives (if shorter) washout from bridging chemotherapy. In some embodiments, the conditioning chemotherapy/lymphodepleting regimen consists of fludarabine intravenous (IV) 25 mg/m²/day on days −4, −3, and −2, and cyclophosphamide IV 900 mg/m²/day on day −2. On day 0, a single infusion of anti-CD19 CAR T cells may be administered. In some embodiments, additional infusions of anti-CD19 CAR T cells may be administered at a later time. In some embodiments, patients achieving complete response to the first infusion may receive a second infusion of anti-CD19 CAR T cells, if progressing following >3 months of remission, provided CD19 expression has been retained and neutralizing antibodies against the CAR are not suspected.

In some embodiments, droplet digital polymerase chain reaction may be used to measure the presence, expansion, and persistence of transduced anti-CD19 CAR+ T cells in the blood. In some embodiments, the procedure is as described in Locke F. L. et al. Mol Ther. 2017:25(1):285-295. In some embodiments, the disclosure provides that CAR T cells may be undetectable at relapse. Median peak CAR T-cell levels may be highest with 1×10⁶ CAR T cells/kg and may be similar between patients who received original vs. revised AE management. In some embodiments, patients achieving CR/CRi had greater median peak expansion than non-responders, as did patients with undetectable vs. detectable MRD. Higher median peak expansion was also observed in patients with grade ≥3 NE vs. those with grade≤2 NE. Some patients who relapse may have detectable CD19-positive cells at relapse or may have no detectable CD19-postive cells. In some embodiments, undetectable MRD, defined as <1 leukemia cell per 10.000 viable cells, may be assessed using flow cytometry (NeoGenomics, Fort Myers, FL) as per the methods described in Borowitz M J, Wood B L, Devidas M, et al. Blood. 2015; 126(8): 964-971; Bruggemann M. et al. Blood Adv. 2017; 1(25):2456-2466; or Gupta S. et al. Leukemia. 2018; 32(6):1370-1379.

In some embodiments, the disclosure provides that peak IL-15 serum levels are lower in patients with grade ≥3 CRS. In some embodiments, the disclosure provides that median peak levels of several pro-inflammatory markers trended higher in patients with grade ≥3 CRS and those with grade ≥3 NE (IFNγ, IL-8, GM-CSF, IL-1RA, CXCL10, MCP-1, Granzyme B. Accordingly, in some embodiments, the disclosure provides a method for predicting whether a patient is going to have grade ≥3 CRS by measuring the peak levels of serum IL-15 and comparing to a reference standard. In some embodiments, the disclosure provides a method for predicting whether a patient is going to have grade 23 CRS and/or grade 23 NE by measuring the peak levels of IFNγ, IL-8, GM-CSF, IL-1RA, CXCL10, MCP-1, and/or Granzyme B and comparing to a reference standard. In some embodiments, the disclosure provides a method for improving anti-CD19 CAR T cell therapy by administering agents that decrease the levels of one or more of these biomarkers.

The reference levels/standards may be established by any method known by one of ordinary skill in the art. They serve to identify thresholds or groups of values (e.g., quartiles) from which a comparison may be made to determine in which group, or above or below which threshold does the measured value (cytokine level, CAR T cell number, etc.) for each subject fall. These groups are established from comparisons of different populations chosen as is typical in the art. Depending on where the measured value falls, one can predict a number of treatment characteristics such as objective response, CRS grade, NE grade, and the like.

In certain embodiments, the cancer may be selected from a tumor derived from acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor. In certain embodiments, the cancer is treated with KTE-X19.

In one embodiment, the method may be used to treat a tumor, wherein the tumor is a lymphoma or a leukemia. Lymphoma and leukemia are cancers of the blood that specifically affect lymphocytes. All leukocytes in the blood originate from a single type of multipotent hematopoietic stem cell found in the bone marrow. This stem cell produces both myeloid progenitor cells and lymphoid progenitor cell, which then give rise to the various types of leukocytes found in the body. Leukocytes arising from the myeloid progenitor cells include T lymphocytes (T cells), B lymphocytes (B cells), natural killer cells, and plasma cells. Leukocytes arising from the lymphoid progenitor cells include megakaryocytes, mast cells, basophils, neutrophils, cosinophils, monocytes, and macrophages. Lymphomas and leukemias may affect one or more of these cell types in a patient. In certain embodiments, the tumor is treated with KTE-X19.

In general, lymphomas may be divided into at least two sub-groups: Hodgkin lymphoma and non-Hodgkin lymphoma. Non-Hodgkin Lymphoma (NHL) is a heterogeneous group of cancers originating in B lymphocytes, T lymphocytes or natural killer cells. In the United States, B cell lymphomas represent 80-85% of cases reported. In 2013 approximately 69.740 new cases of NHL and over 19.000 deaths related to the disease were estimated to occur. Non-Hodgkin lymphoma is the most prevalent hematological malignancy and is the seventh leading site of new cancers among men and women and account for 4% of all new cancer cases and 3% of deaths related to cancer. In certain embodiments, the lymphoma is treated with KTE-X19.

Diffuse large B cell lymphoma (DLBCL) is the most common subtype of NHL, accounting for approximately 30% of NHL cases. There are approximately 22,000 new diagnoses of DLBCL in the United States each year. It is classified as an aggressive lymphoma with the majority of patients cured with conventional chemotherapy (NCCN guidelines NHL 2014). First line therapy for DLBCL typically includes an anthracycline-containing regimen with rituximab, such as R-CHOP (rituximab, cyclophosphamide, doxorubicin, vincristine, and prednisone), which has an objective response rate of about 80% and a complete response rate of about 50%, with about one-third of patients have refractory disease to initial therapy or relapse after R-CHOP. For those patients who relapse after response to first line therapy, approximately 40-60% of patients may achieve a second response with additional chemotherapy. The standard of care for second-line therapy for autologous stem cell transplant (ASCT) eligible patients includes rituximab and combination chemotherapy such as R-ICE (rituximab, ifosfamide, carboplatin, and etoposide) and R-DHAP (rituximab, dexamethasone, cytarabine, and cisplatin), which each have an objective response rate of about 63% and a complete response rate of about 26%. Patients who respond to second line therapy and who are considered fit enough for transplant receive consolidation with high-dose chemotherapy and ASCT, which is curative in about half of transplanted patients Patients who failed ASCT have a very poor prognosis and no curative options. Primary mediastinal large B cell lymphoma (PMBCL) has distinct clinical, pathological, and molecular characteristics compared to DLBCL. PMBCL is thought to arise from thymic (medullary) B cells and represents approximately 3% of patients diagnosed with DLBCL. PMBCL is typically identified in the younger adult population in the fourth decade of life with a slight female predominance. Gene expression profiling suggests deregulated pathways in PMBCL overlap with Hodgkin lymphoma. Initial therapy of PMBCL generally includes anthracycline-containing regimens with rituximab, such as infusional dose-adjusted etoposide, doxorubicin, and cyclophosphamide with vincristine, prednisone, and rituximab (DA-EPOCH-R), with or without involved field radiotherapy. Follicular lymphoma (FL), a B cell lymphoma, is the most common indolent (slow-growing) form of NHL, accounting for approximately 20% to 30% of all NHLs. Some patients with FL will transform (TFL) histologically to DLBCL which is more aggressive and associated with a poor outcome. Histological transformation to DLBCL occurs at an annual rate of approximately 3% for 15 years with the risk of transformation continuing to drop in subsequent years. The biologic mechanism of histologic transformation is unknown. Initial treatment of TFL is influenced by prior therapies for follicular lymphoma but generally includes anthracycline-containing regimens with rituximab to eliminate the aggressive component of the disease. Treatment options for relapsed/refractory PMBCL and TFL are similar to those in DLBCL. Given the low prevalence of these diseases, no large prospective randomized studies in these patient populations have been conducted. Patients with chemotherapy refractory disease have a similar or worse prognosis to those with refractory DLBCL. As an example, subjects who have refractory, aggressive NHL (e.g., DLBCL, PMBCL and TFL) have a major unmet medical need and further research with novel treatments are warranted in these populations. In certain embodiments, the DLBCL is treated with KTE-X19.

The CAR T cell treatment of the disclosure may be administered as a first line of treatment or a second or later line of treatment. In some embodiments, the CAR T cell treatment is administered as a third line, fourth line, fifth line and so on and so forth. The lines of prior therapy may be any prior anti-cancer therapy, including, but not limited to Bruton Tyrosine Kinase inhibitor (BTKi), check-point inhibitors (e.g., anti-PD1 antibodies, pembrolizumab (Keytruda). Cemiplimab (Libtayo), nivolumab (Opdivo); anti-PD-L1 antibodies, Atezolizumab (Tecentriq), Avelumab (Bavencio), Durvalumab (Imfinzi); anti-CTLA-4 antibodies, Ipilimumab (Yervoy)), anti-CD 19 antibodies (e.g. blinatumomab), anti-CD52 antibodies (e.g., alentuzumab); allogeneic stem cell transplantation, anti-CD20 antibodies (e.g., rituximab), systemic chemotherapy, rituximab, anthracycline, ofatumumab, and combination thereof. The prior therapies may also be used in combination with the CD19 CAR T therapies of the application. In one aspect, the eligible patients may have refractory disease to the most recent therapy or relapse within 1 year after autologous hematopoietic stem cell transplantation (HSCT/ASCT). The CAR T cell treatment may be administered to patients that have or suspect to have cancers that are refractory and/or that relapsed to one or more lines of previous therapy. The cancer may be refractory to first-line therapy (i.e., primary refractory) or refractory to one or more lines of therapy. The cancer may have relapsed at twelve months after first remission, relapsed or refractory after two or more lines of prior therapy, or relapsed after HSCT/ASCT. In some embodiments, the cancer is refractory to ibrutinib or acalabrutinib. In some embodiments, the cancer is NHL, and the disease must have been primary refractory, R/R after two or more lines of systemic therapy, or R/R after autologous or allogeneic stem cell transplantation ≥100 days prior to enrollment in CAR T cell therapy and off immunosuppressive medications for ≥4 weeks. In certain embodiments, the CAR T cell therapy is KTE-X19.

Accordingly, the method may be used to treat a lymphoma or a leukemia, wherein the lymphoma or leukemia is a B cell malignancy. Examples of B cell malignancies include, but are not limited to, Non-Hodgkin's Lymphomas (NHL), Small lymphocytic lymphoma (SLL/CLL). Mantle cell lymphoma (MCL), FL. Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, and Lymphoblastic lymphoma. In some aspects, the lymphoma or leukemia is selected from B-cell chronic lymphocytic leukemia/small cell lymphoma, B-cell prolymphocytic leukemia, lymphoplasmacytic lymphoma (e.g., Waldenström macroglobulinemia), splenic marginal zone lymphoma, hairy cell leukemia, plasma cell neoplasms (e.g., plasma cell myeloma (i.e., multiple myeloma), or plasmacytoma), extranodal marginal zone B cell lymphoma (e.g., MALT lymphoma), nodal marginal zone B cell lymphoma, follicular lymphoma (FL), transformed follicular lymphoma (TFL), primary cutaneous follicle center lymphoma, mantle cell lymphoma, diffuse large B cell lymphoma (DLBCL). Epstein-Barr virus-positive DLBCL, lymphomatoid granulomatosis, primary mediastinal (thymic) large B-cell lymphoma (PMBCL), Intravascular large B-cell lymphoma, ALK+large B-cell lymphoma, plasmablastic lymphoma, primary effusion lymphoma, large B-cell lymphoma arising in HHV8-associated multicentric Castleman's disease, Burkitt lymphoma/leukemia, T-cell prolymphocytic leukemia, T-cell large granular lymphocyte leukemia, aggressive NK cell leukemia, adult T-cell leukemia/lymphoma, extranodal NK/T-cell lymphoma, enteropathy-associated T-cell lymphoma, Hepatosplenic T-cell lymphoma, blastic NK cell lymphoma. Mycosis fungoides/Sezary syndrome, Primary cutaneous anaplastic large cell lymphoma, Lymphomatoid papulosis, Peripheral T-cell lymphoma, Angioimmunoblastic T cell lymphoma, Anaplastic large cell lymphoma, B-lymphoblastic leukemia/lymphoma. B-lymphoblastic leukemia/lymphoma with recurrent genetic abnormalities, T-lymphoblastic leukemia/lymphoma, and Hodgkin lymphoma. In some aspect, the cancer is refractory to one or more prior treatments, and/or the cancer has relapsed after one or more prior treatments. In certain embodiments, the leukemia or lymphoma is treated with KTE-X19.

In one embodiment, the cancer is selected from follicular lymphoma, transformed follicular lymphoma, diffuse large B cell lymphoma, and primary mediastinal (thymic) large B-cell lymphoma. In another embodiment, the cancer is diffuse large B cell lymphoma. In some embodiment, the cancer is refractory to or the cancer has relapsed following one or more of chemotherapy, radiotherapy, immunotherapy (including a T cell therapy and/or treatment with an antibody or antibody-drug conjugate), an autologous stem cell transplant, or any combination thereof. In one embodiment, the cancer is refractory diffuse large B cell lymphoma. In certain embodiments, the cancer is treated with KTE-X19.

In some embodiments, the CAR T cell treatment is KTE-X19 and the cancer is selected from MCL, ALL, CLL, and SLL. In some embodiments, the CAR T cell treatment is KTE-X19 and the cancer is NHL. In some embodiments, the cancer is selected from diffuse large B cell lymphoma not otherwise specified (DLBCL NOS), primary mediastinal large B cell lymphoma, Burkitt lymphoma (BL), Burkitt-like lymphoma or unclassified B cell lymphomas intermediate between DLBCL and BL. In some embodiments, the cancer is relapsed/refractory. In some embodiments, the KTE-X19 treatment is administered as first line, second line, or after 1 or more prior lines of therapy. In some embodiments, the patient is a pediatric patient, an adolescent patient, an adult patient, less than 65 years old, more than 65 years old, or any other age group.

In some embodiment, compositions comprising immune cells disclosed herein may be administered in conjunction with any number of additional therapeutic agents. In one embodiment, the additional therapeutic agent is administered concurrently with the T cell therapy. In one embodiment, the additional therapeutic agent is administered prior to, during, and/or after T cell therapy. In one embodiment, the one or more additional therapeutic agents is administered prophylactically. In one aspect, the compositions comprising the immune cells are administered in conjunction with agents for management of adverse events (many of which are described elsewhere in this application, including the Examples section). These agents may manage one or more of the signs and symptoms of adverse reactions, such as fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia.

Examples of such agents include, without limitation, tocilizumab, steroids (e.g., methylprednisolone), rabbit anti-thymocyte globulin. In some aspect, Vancomycin and aztreonam (each 1 gm IV twice daily) may be administered for non-neutropenic fever. In some aspects, the method further comprises administering a non-sedating, anti-seizure medicine for seizure prophylaxis; administering at least one of erythropoietin, darbepoetin alfa, platelet transfusion, filgrastim, or pegfilgrastim; and/or administering tocilizumab, siltuximab. In one aspect, the agent is a CSF family member such as GM-CSF (Granulocyte-macrophage colony-stimulating factor, also known as CSF2). GM-CSF may be produced by a number of haemopoictic and nonhaemopoietic cell types upon stimulation, and it may activate/‘prime’ myeloid populations to produce inflammatory mediators, such as TNF and interleukin 1β (IL1β). In some embodiments, the GM-CSF inhibitor is an antibody that binds to and neutralizes circulating GM-CSF. In some embodiments, the antibody is selected from Lenzilumab; namilumab (AMG203); GSK3196165/MOR103/Otilimab (GSK/MorphoSys), KB002 and KB003 (KaloBios), MT203 (Micromet and Nycomed), and MORAb-022/gimsilumab (Morphotek). In some embodiments, the antibody is a biosimilar of the same. In some embodiments, the antagonist is E21R, a modified form of GM-CSF that antagonizes the function of GM-CSF. In some embodiments, the inhibitor/antagonist is a small molecule. In one embodiment, the CSF family member is M-CSF (also known as macrophage colony-stimulating factor or CSF1). Non-limiting examples of agents that inhibit or antagonize CSF1 include small molecules, antibodies, chimeric antigen receptors, fusion proteins, and other agents. In one embodiment, the CSF1 inhibitor or antagonist is an anti-CSF1 antibody. In one embodiment, the anti-CSF1 antibody is selected from those made by Roche (e.g., RG7155), Pfizer (PD-0360324), Novartis (MCS110/lacnotuzumab), or a biosimilar version of any one of the same. In some embodiments, the inhibitor or antagonist inactivates the activity of either the GM-CSF-R-alpha (aka CSF2R) or CSF1R receptors. In some embodiments, the inhibitor is selected from Mavrilimumab (formerly CAM-3001), a fully human GM-CSF Receptor α monoclonal antibody currently being developed by MedImmune, Inc.; cabiralizumab (Five Prime Therapeutics); LY3022855 (IMC-CS4)(Eli Lilly), Emactuzumab, also known as RG7155 or RO5509554; FPA008, a humanized mAb (Five Prime/BMS); AMG820 (Amgen); ARRY-382 (Array Biopharma); MCS110 (Novartis); PLX3397 (Plexxikon); ELB041/AFS98/TG3003 (ElsaLys Bio. Transgene), SNDX-6352 (Syndax). In some embodiments, the inhibitor or antagonist is expressed in CAR-T cells. In some embodiments, the inhibitor is a small molecule (e.g. heteroaryl amides, quinolinone series, pyrido-pyrimide series); BLZ945 (Novartis), PLX7486, ARRY-382, Pexidrtinib (also known as PLX3397) or 5-((5-chloro-1H-pyrrolo[2,3-b]pyridin-3-yl)methyl)-N-06-(trifluoromethyl)pyridin-3-yl)methyl)pyridin-2-amine; GW 2580 (CAS 870483-87-7), KÏ20227 (CAS 623142-96-1), AC708 by Ambit Siosciences, or any CSF1R inhibitor listed in Cannarile et al. Journal for ImmunoTherapy of Cancer 2017, 5:53 and US20180371093, incorporated herein by reference for the inhibitors they disclose. Additional neutralizing antibodies to GM-CSF or its receptor have been described in the art, including in, for example, “GM-CSF as a target in inflammatory/autoimmune disease: current evidence and future therapeutic potential” Hamilton, J. A. Expert Rev. Clin. Immunol., 2015; and “Targeting GM-CSF in inflammatory diseases” Wicks, I. P., Roberts, A. W. Nat. Rev. Rheumatol., 2016. In other embodiments, the agent is an anti-IL6 or anti-IL-6receptor blocking agent, including tocilizumab and siltuximab.

In one aspect, the therapeutic agent is a chemotherapeutic agent. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa: ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine. 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotricthylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. In some aspects, compositions comprising CAR- and/or TCR-expressing immune effector cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles. 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP. i.e., Cyclophosphamide (Cytoxan®), Doxorubicin (hydroxydoxorubicin). Vincristine (Oncovin®), and Prednisone.

The (chemo) therapeutic agent may be administered at the same time or within one week after the administration of the engineered cell or nucleic acid. In other aspects, the (chemo) therapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In some aspects, the (chemo) therapeutic agent is administered at least 1 month before administering the cell or nucleic acid. In some aspects, the methods further comprise administering two or more chemotherapeutic agents.

A variety of additional therapeutic agents may be used in conjunction/combination with the compositions or agents/treatments described herein. For example, potentially useful additional therapeutic agents include PD-1 inhibitors such as nivolumab (OPDIVO®), pembrolizumab (KEYTRUDA®), pembrolizumab, pidilizumab (CureTech), and atezolizumab (Roche), tocilizumab (with and without corticosteroids), inhibitors of GM-CSF, CSF1, GM-CSFR, or CSF1R GM-CSF, CSF1, GM-CSFR, or CSF1R (anti-CSF1 antibody is selected from those made by Roche (e.g., RG7155), Pfizer (PD-0360324), Novartis (MCS110/laenotuzumab), Mavrilimumab (formerly CAM-3001), a fully human GM-CSF Receptor α monoclonal antibody currently being developed by MedImmune, Inc.; cabiralizumab (Five Prime Therapeutics); LY3022855 (IMC-CS4)(Eli Lilly), Emactuzumab, also known as RG7155 or RO5509554; FPA008, a humanized mAb (Five Prime/BMS): AMG820 (Amgen); ARRY-382 (Array Biopharma); MCS110 (Novartis); PLX3397 (Plexxikon); ELB041/AFS98/TG3003 (ElsaLys Bio, Transgene), SNDX-6352 (Syndax). In some aspects, the inhibitor or antagonist is expressed in CAR-T cells. In some aspects, the inhibitor is a small molecule (e.g. heteroaryl amides, quinolinone series, pyrido-pyrimide series); BLZ945 (Novartis), PLX7486, ARRY-382, Pexidrtinib (also known as PLX3397) or 5-((5-chloro-1H-pyrrolo[2.3-b]pyridin-3-yl)methyl)-N-06-(trifluoromethyl)pyridin-3-yl)methyl)pyridin-2-amine; GW 2580 (CAS 870483-87-7), KÏ20227 (CAS 623142-96-1). AC708 by Ambit Siosciences, or any CSF1R inhibitor listed in Cannarile et al. Journal for ImmunoTherapy of Cancer 2017, 5:53 and US20180371093, incorporated herein by reference for the inhibitors they disclose. Additional neutralizing antibodies to GM-CSF or its receptor have been described in the art). Additional therapeutic agents suitable for use in combination with the compositions or agents/treatments and methods disclosed herein include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, lenalidomide, axitinib, masitinib, pazopanib, sunitinib, sorafenib, tocilizumab, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

The composition or agents/treatments comprising immune cells are, or may be, administered with an anti-inflammatory agent. Anti-inflammatory agents or drugs may include, but are not limited to, steroids and glucocorticoids (including betamethasone, budesonide, dexamethasone, hydrocortisone corticosteroid, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium. Cox-2 inhibitors, and sialylates. Exemplary analgesics include acetaminophen, oxycodone, tramadol of proporxyphene hydrochloride.

Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

The compositions or agents/treatments described herein may be administered in conjunction with a cytokine and/or a cytokine modulator as an additional therapeutic agent. Examples of cytokines are lymphokines, monokines, and traditional polypeptide hormones. Included among the cytokines are growth hormones such as human growth hormone. N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO, Epogen®, Procrit®); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. In one embodiment, the compositions described herein are administered in conjunction with a steroid or corticosteroid.

Corticosteroid treatment may be used for treatment of adverse events. Corticosteroids (or any other steroids, as well as any other treatment for adverse events) may be used prophylactically, before any symptoms of adverse events are detected and/or after detection of adverse events. They may be administered one or more days prior to T cell administration, on the day of T cell administration (before, after, and/or during T cell administration), and/or after T cell administration. They may be administered prior to, during, or after conditioning therapy. Any corticosteroid may be appropriate for this use. In one embodiment, the corticosteroid is dexamethasone. In some embodiments, the corticosteroid is methylprednisolone. In some embodiments, the two are administered in combination. In some embodiments, glucocorticoids include synthetic and non-synthetic glucocorticoids. Exemplary glucocorticoids include, but are not limited to: alelomethasones, algestones, beclomethasones (e.g. beclomethasone dipropionate), betamethasones (e.g. betamethasone 17 valerate, betamethasone sodium acetate, betamethasone sodium phosphate, betamethasone valerate), budesonides, clobetasols (e.g. clobetasol propionate), clobetasones, clocortolones (e.g. clocortolone pivalate), cloprednols, corticosterones, cortisones and hydrocortisones (e.g. hydrocortisone acetate), cortivazols, deflazacorts, desonides, desoximethasones, dexamethasones (e.g. dexamethasone 21-phosphate, dexamethasone acetate, dexamethasone sodium phosphate), diflorasones (e.g. diflorasone diacetate), diflucortolones, difluprednates, enoxolones, fluazacorts, flucloronides, fludrocortisones (e.g., fludrocortisone acetate), flumethasones (e.g. flumethasone pivalate), flunisolides, fluocinolones (e.g. fluocinolone acetonide), fluocinonides, fluocortins, fluocortolones, fluorometholones (e.g. fluorometholone acetate), fluperolones (e.g., fluperolone acetate), fluprednidenes, flupredni solones, flurandrenolides, fluticasones (e.g. fluticasone propionate), formocortals, halcinonides, halobetasols, halometasones, halopredones, hydrocortamates, hydrocortisones (e.g. hydrocortisone 21-butyrate, hydrocortisone aceponate, hydrocortisone acetate, hydrocortisone buteprate, hydrocortisone butyrate, hydrocortisone cypionate, hydrocortisone hemisuccinate, hydrocortisone probutate, hydrocortisone sodium phosphate, hydrocortisone sodium succinate, hydrocortisone valerate), loteprednol etabonate, mazipredones, medrysones, meprednisones, methylpredni solones (methylprednisolone aceponate, methylprednisolone acetate, methylprednisolone hemisuccinate, methylprednisolone sodium succinate), mometasones (e.g., mometasone furoate), paramethasones (e.g., paramethasone acetate), prednicarbates, prednisolones (e.g. prednisolone 25-diethylaminoacetate, prednisolone sodium phosphate, prednisolone 21-hemisuccinate, prednisolone acetate; prednisolone farnesylate, prednisolone hemisuccinate, prednisolone-21 (beta-D-glucuronide), prednisolone metasulphobenzoate, prednisolone steaglate, prednisolone tebutate, prednisolone tetrahydrophthalate), prednisones, prednivals, prednylidenes, rimexolones, tixocortols, triamcinolones (e.g. triamcinolone acetonide, triamcinolone benetonide, triamcinolone hexacetonide, triamcinolone acetonide 21 palmitate, triamcinolone diacetate). These glucocorticoids and the salts thereof are discussed in detail, for example, in Remington's Pharmaceutical Sciences, A. Osol, ed., Mack Pub. Co., Easton, Pa. (16th ed. 1980) and Remington: The Science and Practice of Pharmacy, 22nd Edition, Lippincott Williams & Wilkins, Philadelphia, Pa. (2013) and any other editions, which are hereby incorporated by reference. In some embodiments, the glucocorticoid is selected from among cortisones, dexamethasones, hydrocortisones, methylprednisolones, prednisolones and prednisones. In an embodiment, the glucocorticoid is dexamethasone. In other embodiments, the steroid is a mineralcorticoid. Any other steroid may be used in the methods provided herein.

The one or more corticosteroids may be administered at any dose and frequency of administration, which may be adjusted to the severity/grade of the adverse event (e.g., CRS and NE). In another embodiment, corticosteroid administration comprises oral or IV dexamethasone 10 mg, 1-4 times per day. Another embodiment, sometimes referred to as “high-dose” corticosteroids, comprises administration of IV methylprednisone 1 g per day alone, or in combination with dexamethasone. In some embodiments, the one or more cortico steroids are administered at doses of 1-2 mg/kg per day.

The corticosteroid may be administered in any amount that is effective to ameliorate one or more symptoms associated with the adverse events, such as with the CRS or neurotoxicity. The corticosteroid, e.g., glucocorticoid, can be administered, for example, at an amount between at or about 0.1 and 100 mg, per dose. 0.1 to 80 mg. 0.1 to 60 mg, 0.1 to 40 mg, 0.1 to 30 mg, 0.1 to 20 mg. 0.1 to 15 mg. 0.1 to 10 mg, 0.1 to 5 mg. 0.2 to 40 mg. 0.2 to 30 mg, 0.2 to 20 mg. 0.2 to 15 mg, 0.2 to 10 mg, 0.2 to 5 mg, 0.4 to 40 mg, 0.4 to 30 mg, 0.4 to 20 mg, 0.4 to 15 mg, 0.4 to 10 mg. 0.4 to 5 mg, 0.4 to 4 mg, 1 to 20 mg, 1 to 15 mg or 1 to 10 mg, to a 70 kg adult human subject. Typically, the corticosteroid, such as a glucocorticoid is administered at an amount between at or about 0.4 and 20 mg, for example, at or about 0.4 mg, 0.5 mg, 0.6 mg, 0.7 mg, 0.75 mg, 0.8 mg, 0.9 mg, 1 mg, 2 mg, 3 mg, 4 mg. 5 mg, 6 mg, 7 mg, 8 mg, 9 mg, 10 mg, 11 mg. 12 mg, 13 mg, 14 mg. 15 mg, 16 mg. 17 mg. 18 mg, 19 mg or 20 mg per dose, to an average adult human subject.

In some embodiments, the corticosteroid may be administered, for example, at a dosage of at or about 0.001 mg/kg (of the subject), 0.002 mg/kg. 0.003 mg/kg, 0.004 mg/kg, 0.005 mg/kg. 0.006 mg/kg, 0.007 mg/kg, 0.008 mg/kg, 0.009 mg/kg, 0.01 mg/kg. 0.015 mg/kg, 0.02 mg/kg. 0.025 mg/kg. 0.03 mg/kg, 0.035 mg/kg. 0.04 mg/kg. 0.045 mg/kg. 0.05 mg/kg, 0.055 mg/kg. 0.06 mg/kg, 0.065 mg/kg. 0.07 mg/kg. 0.075 mg/kg, 0.08 mg/kg, 0.085 mg/kg. 0.09 mg/kg, 0.095 mg/kg. 0.1 mg/kg, 0.15 mg/kg, 0.2 mg/kg, 0.25 mg/kg, 0.30 mg/kg, 0.35 mg/kg, 0.40 mg/kg, 0.45 mg/kg. 0.50 mg/kg. 0.55 mg/kg, 0.60 mg/kg, 0.65 mg/kg, 0.70 mg/kg. 0.75 mg/kg. 0.80 mg/kg, 0.85 mg/kg, 0.90 mg/kg, 0.95 mg/kg, 1 mg/kg, 1.05 mg/kg, 1.1 mg/kg, 1.15 mg/kg, 1.20 mg/kg, 1.25 mg/kg, 1.3 mg/kg, 1.35 mg/kg or 1.4 mg/kg, to an average adult human subject, typically weighing about 70 kg to 75 kg.

Generally, the dose of corticosteroid administered is dependent upon the specific corticosteroid, as a difference in potency exists between different corticosteroids. It is typically understood that drugs vary in potency, and that doses can therefore vary, in order to obtain equivalent effects. Equivalence in terms of potency for various glucocorticoids and routes of administration, is well known. Information relating to equivalent steroid dosing (in a non-chronotherapeutic manner) may be found in the British National Formulary (BNF) 37, March 1999.

In some embodiments, the adverse events/reactions may be chosen from one or more of the following:

Adverse Event/Reaction

Blood and Lymphatic System Disorders
Coagulopathy ^{a}
Cardiac Disorders
Tachycardias ^{b}
Bradycardias ^{c}
Non-ventricular Arrhythmias ^{d}
Gastrointestinal Disorders
Nausea
Constipation
Diarrhea
Abdominal pain ^{e}
Oral pain ^{f}
Vomiting ^{g}
Dysphagia
Pyrexia
Fatigue ^{h}
Chills
Edema ⁱ
Dry mouth
Pain ^{j}
Immune System Disorders
Cytokine release syndrome
Hypogammaglobulinemia ^{k}
Infections and Infestations
Infection - pathogen unspecified
Viral infections
Bacterial infections
Immune System Disorders
Cytokine release syndrome
Hypogammaglobulinemia ^{k}
Infections and Infestations
Infection - pathogen unspecified
Viral infections
Bacterial infections
Metabolism and nutrition disorders
Decreased appetite
Musculoskeletal pain ^{l}
Motor dysfunction ^{m}
Psychiatric Disorders
Nervous System Disorders
Encephalopathy ⁿ
Tremor
Headache ^{o}
Aphasia ^{p}
Dizziness ^{q}
Neuropathy ^{r}
Insomnia
Delirium ^{s}
Anxiety
Renal and Urinary Disorders
Renal insufficiency ^{t}
Urine output decreased ^{u}
Hypoxia
Cough ^{v}
Dyspnea ^{w}
Metabolism and nutrition disorders
Decreased appetite
Musculoskeletal pain ^{l}
Motor dysfunction ^{m}
Psychiatric Disorders
Nervous System Disorders
Encephalopathy ⁿ
Tremor
Headache ^{o}
Aphasia ^{p}
Dizziness ^{q}
Neuropathy ^{r}
Insomnia
Delirium ^{s}
Anxiety
Pleural effusion
Skin and Subcutaneous Tissue Disorders
Rash ^{x}
Vascular Disorders
Hypotension ^{y}
Hypertension
Thrombosis ^{z}
Hemorrage

Other adverse reactions include: Gastrointestinal disorders: dry mouth; Infections and infestations disorders: fungal infection; Metabolism and nutrition disorders: dehydration; Nervous system disorders: ataxia, seizure, increased intracranial pressure; Respiratory, thoracic and mediastinal disorders: respiratory failure, pulmonary edema; Skin and subcutaneous tissue disorders: rash; Vascular disorders: hemorrhage.

In one embodiment, cytokine release syndrome symptoms include but are not limited to, fever, rigors, fatigue, anorexia, myalgias, arthalgias, nausea, vomiting, headache, rash, diarrhoea, tachypnea, hypoxemia, tachycardia, hypotension, widened pulse pressure, early increased cardiac output, late diminished cardiac output, hallucinations, tremor, altered gait, seizures and death. In one embodiment, a method for grading CRS is described in Neelapu et al., Nat Rev Clin Oncol. 15(1):47-62 (2018) and Lee, et al., Blood 2014; 124:188-195. In one embodiment, Neurotoxicity/Neurologic events may be graded by the method described in Lee, et al, Blood 2014; 124: 188-195.

In some embodiments, the adverse events are managed with tocilizumab (or another anti-IL6/IL6R agent/antagonist), a corticosteroid therapy, or an anti-seizure medicine for toxicity prophylaxis. In some embodiments, the adverse events are managed by one or more agent(s) selected from inhibitors of GM-CSF, CSF1, GM-CSFR, or CSF1R, anti-thymocyte globulin, lenzilumab, mavrilimumab, cytokines, and anti-inflammatory agents.

In some embodiments, the present disclosure provides methods of preventing the development or reducing the severity of adverse reactions to the T cell treatments of the disclosure. In some embodiments, the cell therapy is administered in with one or more agents that prevents, delays the onset of, reduces the symptoms of, treats the adverse events, which include cytokine release syndromes and neurologic toxicity. In one embodiment, the agent has been described above. In other embodiments, the agent is described below. In some embodiments, the agent is administered by one of the methods and doses described elsewhere in the specification, before, after, or concurrently with the administration of the cells. In one embodiment, the agent(s) are administered to a subject that may be predisposed to the disease but has not yet been diagnosed with the disease.

In this respect, the disclosed method may comprise administering a “prophylactically effective amount” of tocilizumab, of a corticosteroid therapy, and/or of an anti-seizure medicine for toxicity prophylaxis. In some embodiments, the method comprises administering inhibitors of GM-CSF, CSF1, GM-CSFR, or CSF1R, lenzilumab, mavrilimumab, cytokines, and/or anti-inflammatory agents. The pharmacologic and/or physiologic effect may be prophylactic, i.e., the effect completely or partially prevents a disease or symptom thereof. A “prophylactically effective amount” may refer to an amount effective, at dosages and for periods of time necessary, to achieve a desired prophylactic result (e.g., prevention of onset of adverse reactions).

In some embodiments, the method comprises management of adverse reactions in any subject. In some embodiments, the adverse reaction is selected from the group consisting of cytokine release syndrome (CRS), a neurologic toxicity, a hypersensitivity reaction, a serious infection, a cytopenia and hypogammaglobulinemia. In some embodiments, the signs and symptoms of adverse reactions are selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia. In some embodiments, the patient has been identified and selected based on one or more of the biomarkers of adverse events.

In some embodiments, the method comprises preventing or reducing the severity of CRS in a chimeric receptor treatment. In some embodiments, the engineered CAR T cells are deactivated after administration to the patient. In some embodiments, the method comprises identifying CRS based on clinical presentation. In some embodiments, the method comprises evaluating for and treating other causes of fever, hypoxia, and hypotension. Patients who experience ≥Grade 2 CRS (e.g., hypotension, not responsive to fluids, or hypoxia requiring supplemental oxygenation) should be monitored with continuous cardiac telemetry and pulse oximetry. In some embodiments, for patients experiencing severe CRS, consider performing an echocardiogram to assess cardiac function. For severe or life-threatening CRS, intensive care supportive therapy may be considered. In some embodiments, the method comprises monitoring patients at least daily for 7 days at the certified healthcare facility following infusion for signs and symptoms of CRS. In some embodiments, the method comprises monitoring patients for signs or symptoms of CRS for 4 weeks after infusion. In some embodiments, the method comprises counseling patients to seek immediate medical attention should signs or symptoms of CRS occur at any time. In some embodiments, the method comprises instituting treatment with supportive care, tocilizumab or tocilizumab and corticosteroids as indicated at the first sign of CRS.

In some embodiments, the method comprises monitoring patients for signs and symptoms of neurologic toxicities. In some embodiments, the method comprises ruling out other causes of neurologic symptoms. Patients who experience≥Grade 2 neurologic toxicities should be monitored with continuous cardiac telemetry and pulse oximetry. Provide intensive care supportive therapy for severe or life-threatening neurologic toxicities. In some embodiments, the symptom of neurologic toxicity is selected from encephalopathy, headache, tremor, dizziness, aphasia, delirium, insomnia, and anxiety.

In some embodiments, the cell treatment is administered before, during/concurrently, and/or after the administration of one or more agents (e.g., steroids) or treatments (e.g., debulking) that treat and or prevent (are prophylactic) one or more symptoms of adverse events. A “prophylactically effective amount” refers to an amount effective, at dosages and for periods of time necessary, to achieve the desired prophylactic result. In one embodiment, a prophylactically effective amount is used in subjects prior to or at an earlier stage of disease. In one embodiment, the prophylactically effective amount will be less than the therapeutically effective amount. In one embodiment, the adverse event treatment or prophylaxis is administered to any patient that will receive, is receiving, or has received cell therapy. In some embodiments, the method of managing adverse events comprises monitoring patients at least daily for 7 days at the certified healthcare facility following infusion for signs and symptoms of neurologic toxicities. In some embodiments, the method comprises monitoring patients for signs or symptoms of neurologic toxicities and/or CRS for 4 weeks after infusion.

In some embodiments, the disclosure provides two methods of managing adverse events in subjects receiving CAR T cell treatment with steroids and anti-IL6/anti-IL-6R antibody/ies. In one embodiment, the disclosure provides a method of adverse event management whereby corticosteroid therapy is initiated for management of all cases of grade 1 CRS if there was no improvement after 3 days and for all grade ≥1 neurologic events. In one embodiment, tocilizumab is initiated for all cases of grade 1 CRS if there is no improvement after 3 days and for all grade ≥2 neurologic events. In one embodiment, the disclosure provides a method of reducing overall steroid exposure in patients receiving adverse event management after CAR T cell administration, the method comprising initiation of corticosteroid therapy for management of all cases of grade 1 CRS if there was no improvement after 3 days and for all grade ≥1 neurologic events and/or initiation of tocilizumab for all cases of grade 1 CRS if there is no improvement after 3 days and for all grade ≥2 neurologic events. In one embodiment, the corticosteroid and tocilizumab are administering in a regimen selected from those exemplified the Examples section. In one embodiment, the disclosure provides that earlier steroid use is not associated with increased risk for severe infection, decreased CAR T-cell expansion, or decreased tumor response.

In one embodiment, the disclosure supports the safety of levetiracetam prophylaxis in CAR T cell cancer treatment. In one embodiment, the cancer is NHL. In one embodiment, the cancer is R/R LBCL and the patients receive KTE-X19. Accordingly, in one embodiment, the disclosure provides a method of managing adverse events in patients treated with CAR T cells comprising administering to the patient a prophylactic dosage of an anti-seizure medication. In some embodiments, the patients receive levetiracetam (for example, 750 mg orally or intravenous twice daily) starting on day 0 of the CAR T cell treatment (after conditioning) and also at the onset of grade ≥2 neurologic toxicities, if neurologic events occur after the discontinuation of prophylactic levetiracetam. In one embodiment, if a patient does not experience any grade ≥2 neurologic toxicities, levetiracetam is tapered and discontinued as clinically indicated. In one embodiment, levetiracetam prophylaxis is combined with any other adverse event management protocol.

In one embodiment, patients may receive levetiracetam (750 mg oral or intravenous twice daily) starting on day 0. At the onset of grade ≥2 neurologic events, levetiracetam dose is increased to 1000 mg twice daily. If a patient did not experience any grade ≥2 neurologic event, levetiracetam is tapered and discontinued as clinically indicated. Patients also receive tocilizumab (8 mg/kg IV over 1 hour [not to exceed 800 mg]) on day 2. Further tocilizumab (±corticosteroids) may be recommended at the onset of grade 2 CRS in patients with comorbidities or older age, or otherwise in case of grade ≥3 CRS. For patients experiencing grade ≥2 neurologic events, tocilizumab is initiated, and corticosteroids are added for patients with comorbidities or older age, or if there is any occurrence of a grade ≥3 neurologic event with worsening symptoms despite tocilizumab use.

In one embodiment, the disclosure provides that prophylactic steroid use appears to reduce the rate of severe CRS and NEs to a similar extent as early steroid use administration. Accordingly, the disclosure provides a method for adverse event management in CAR T-cell therapy wherein patients receive dexamethasone 10 mg PO on Days 0 (prior to infusion), 1, and 2. Steroids may also administered starting at Grade 1 NE, and for Grade 1 CRS when no improvement is observed after 3 days of supportive care. Tocilizumab may also administered for Grade ≥ 1 CRS if no improvement is observed after 24 hours of supportive care. In one embodiment, the disclosure provides that adverse event management of CAR T-cell therapy with an antibody that neutralizes and/or depletes GM-CSF prevents or reduces treatment-related CRS and/or NEs in treated patients. In one embodiment, the antibody is lenzilumab.

In some embodiments, the adverse events are managed by the administration of an agent/agents that is/are an antagonist or inhibitor of IL-6 or the IL-6 receptor (IL-6R). In some embodiments, the agent is an antibody that neutralizes IL-6 activity, such as an antibody or antigen-binding fragment that binds to IL-6 or IL-6R. For example, in some embodiments, the agent is or comprises tocilizumab (atlizumab) or sarilumab, anti-IL-6R antibodies. In some embodiments, the agent is an anti-IL-6R antibody described in U.S. Pat. No. 8,562,991. In some cases, the agent that targets IL-6 is an anti-TL-6 antibody, such as siltuximab, elsilimomab. ALD518/BMS-945429, sirukumab (CNTO 136), CPSI-2634, ARGX 109, FE301, FM101, or olokizumab (CDP6038), and combinations thereof. In some embodiments, the agent may neutralize IL-6 activity by inhibiting the ligand-receptor interactions. In some embodiments, the IL-6/IL-6R antagonist or inhibitor is an IL-6 mutein, such as one described in U.S. Pat. No. 5,591,827. In some embodiments, the agent that is an antagonist or inhibitor of IL-6/IL-6R is a small molecule, a protein or peptide, or a nucleic acid.

In some embodiments, other agents that may be used to manage adverse reactions and their symptoms include an antagonist or inhibitor of a cytokine receptor or cytokine. In some embodiments, the cytokine or receptor is IL-10, TL-6, TL-6 receptor, IFNγ, IFNGR, IL-2, IL-2R/CD25, MCP-1, CCR2, CCR4, MIP13, CCR5, TNFalpha, TNFR1, such as TL-6 receptor (IL-6R), IL-2 receptor (IL-2R/CD25), MCP-1 (CCL2) receptor (CCR2 or CCR4), a TGF-beta receptor (TGF-beta I, II, or III). IFN-gamma receptor (IFNGR), MIP1P receptor (e.g., CCR5), TNF alpha receptor (e.g., TNFR1), IL-1 receptor (IL1-Ra/IL-1RP), or IL-10 receptor (IL-10R), IL-1, and IL-1Ralpha/IL-1beta. In some embodiments, the agent comprises situximab, sarilumab, olokizumab (CDP6038), elsilimomab, ALD518/BMS-945429, sirukumab (CNTO 136), CPSI-2634, ARGX 109, FE301, or FM101. In some embodiments, the agent, is an antagonist or inhibitor of a cytokine, such as transforming growth factor beta (TGF-beta), interleukin 6 (TL-6), interleukin 10 (IL-10). IL-2. MIP13 (CCL4), TNF alpha, IL-1, interferon gamma (IFN-gamma), or monocyte chemoattractant protein-I (MCP-1). In some embodiments, the is one that targets (e.g. Inhibits or is an antagonist of) a cytokine receptor, such as TL-6 receptor (IL-6R), IL-2 receptor (IL-2R/CD25), MCP-1 (CCL2) receptor (CCR2 or CCR4), a TGF-beta receptor (TGF-beta I, II, or III), IFN-gamma receptor (IFNGR), MIP1P receptor (e.g., CCR5), TNF alpha receptor (e.g., TNFR1), IL-1 receptor (IL1-Ra/IL-1RP), or IL-10 receptor (IL-10R) and combinations thereof. In some embodiments, the agent is administered by one of the methods and doses described elsewhere in the specification, before, after, or concurrently with the administration of the cells.

In some embodiments, the agent is administered in a dosage amount of from or from about 1 mg/kg to 10 mg/kg, 2 mg/kg to 8 mg/kg. 2 mg/kg to 6 mg/kg. 2 mg/kg to 4 mg/kg or 6 mg/kg to 8 mg/kg, each inclusive, or the agent is administered in a dosage amount of at least or at least about or about 2 mg/kg, 4 mg/kg, 6 mg/kg or 8 mg/kg. In some embodiments, is administered in a dosage amount from about 1 mg/kg to 12 mg/kg, such as at or about 10 mg/kg. In some embodiments, the agent is administered by intravenous infusion. In one embodiment, the agent is tocilizumab. In some embodiments, the (agent(s), e.g., specifically tocilizumab) is/are administered by one of the methods and doses described elsewhere in the specification, before, after, or concurrently with the administration of the cells.

In some embodiments, the method comprises identifying CRS based on clinical presentation. In some embodiments, the method comprises evaluating for and treating other causes of fever, hypoxia, and hypotension. If CRS is observed or suspected, it may be managed according to the recommendations in protocol A, which may also be used in combination with the other treatments of this disclosure, including Neutralization or Reduction of the CSF/CSFR1 Axis. Patients who experience ≥Grade 2 CRS (e.g., hypotension, not responsive to fluids, or hypoxia requiring supplemental oxygenation) should be monitored with continuous cardiac telemetry and pulse oximetry. In some embodiments, for patients experiencing severe CRS, consider performing an echocardiogram to assess cardiac function. For severe or life-threatening CRS, intensive care supportive therapy may be considered. In some embodiments, a biosimilar or equivalent of tocilizumab may be used instead of tocilizumab in the methods disclosed herein. In other embodiments, another anti-IL6R may be used instead of tocilizumab.

In some embodiments, adverse events are managed according to the following protocol (protocol A):

| CRS Grade (a) | Tocilizumab | Corticosteroids |
|---|---|---|
| Grade 1 | N/A | N/A |
| Symptoms require | | |
| symptomatic treatment only | | |
| (e.g., fever, nausea, fatigue, | | |
| headache, myalgia, malaise), | | |
| Grade 2 | Administer tocilizumab (c) | Manage per Grade 3 if no |
| Symptoms require and | 8 mg/kg IV over 1 hour | improvement within 24 hours |
| respond to moderate | (not to exceed 800 mg). | after starting tocilizumab. |
| intervention. | Repeat tocilizumab every | |
| Oxygen requirement less than | 8 hours as needed if not | |
| 40% FiO₂ or hypotension | responsive to IV fluids or | |
| responsive to fluids or | increasing supplemental | |
| low-dose of one vasopressor | oxygen. | |
| or Grade 2 organ toxicity (b). | Limit to a maximum of 3 | |
| | doses in a 24-hour period; | |
| | maximum total of 4 doses if | |
| | no clinical improvement in | |
| | the signs and symptoms of | |
| | CRS. | |
| Grade 3 | Per Grade 2 | Administer |
| Symptoms require and | | methylprednisolone 1 mg/kg |
| respond to aggressive | | IV twice daily or equivalent |
| intervention. | | dexamethasone (e.g., 10 mg |
| Oxygen requirement greater | | IV every 6 hours). |
| than or equal to 40% FiO₂ or | | Continue corticosteroids use |
| hypotension requiring high- | | until the event is Grade 1 or |
| dose or multiple vasopressors | | less, then taper over 3 days. |
| or Grade 3 organ toxicity or | | If not improving, manage as |
| Grade 4 transaminitis. | | Grade 4. |
| Grade 4 | Per Grade 2 | Administer |
| Life-threatening symptoms. | | methylprednisolone 1000 mg |
| Requirements for ventilator | | IV per day for 3 days; if |
| support, continuous | | improves, then manage as |
| veno-venous hemodialysis | | above. |
| (CVVHD) or | | Consider alternate |
| Grade 4 organ toxicity | | immunosuppressants if no |
| (excluding transaminitis). | | improvement or if condition |
| | | worsens. |

(a) Lee D W et al., (2014). Current concepts in the diagnosis and management of cytokine release syndrome. Blood. 2014 Jul. 10; 124 (2): 188-195.
(b) Refer to protocol B for management of neurologic toxicity.
(c) Refer to ACEMTRA ® (tocilizumab) Prescribing Information for details,
https://www.gene.com/download/pdf/actemra_prescribing.pdf (last accessed Oct. 18, 2017).
Initial U.S. approval is indicated to be in 2010.

### Neurologic Toxicity

In some embodiments, the method comprises monitoring patients for signs and symptoms of neurologic toxicities. In some embodiments, the method comprises ruling out other causes of neurologic symptoms. Patients who experience ≥Grade 2 neurologic toxicities should be monitored with continuous cardiac telemetry and pulse oximetry. Provide intensive care supportive therapy for severe or life-threatening neurologic toxicities. Consider non-sedating, anti-seizure medicines (e.g., levetiracetam) for seizure prophylaxis for any ≥Grade 2 neurologic toxicities. The following treatments may be used in combination with the other treatments of this disclosure, including Neutralization or Reduction of the CSF/CSFR1 Axis.

In some embodiments, adverse events are managed according to the following protocol (protocol B):

| Grading | | |
|---|---|---|
| Assessment | Concurrent CRS | No concurrent CRS |
| Grade 2 | Administer tocilizumab per protocol A | Administer dexamethasone |
| | (table above) for management of | 10 mg IV every 6 hours. |
| | Grade 2 CRS. | Continue dexamethasone use |
| | If no improvement within 24 hours after | until the event is Grade 1 or |
| | starting tocilizumab, administer | less, then taper over 3 days. |
| | dexamethasone 10 mg IV every 6 hours if | |
| | not already taking other steroids. | |
| | Continue dexamethasone use until the | |
| | event is Grade 1 or less, then taper over 3 | |
| | days. | |
| | Consider non-sedating, anti-seizure medicines | |
| | (e.g., levetiracetam) for seizure prophylaxis. | |
| Grade 3 | Administer tocilizumab per (protocol A) | Administer dexamethasone 10 |
| | for management of Grade 2 CRS. | mg IV every 6 hours. |
| | In addition, administer dexamethasone 10 | Continue dexamethasone use |
| | mg IV with the first dose of tocilizumab | until the event is Grade 1 or |
| | and repeat dose every 6 hours. Continue | less, then taper over 3 days. |
| | dexamethasone use until the event is | |
| | Grade 1 or less, then taper over 3 days. | |
| | Consider non-sedating, anti-seizure medicines | |
| | (e.g., levetiracetam) for seizure prophylaxis. | |
| Grade 4 | Administer tocilizumab per (protocol A) | Administer |
| | for management of Grade 2 CRS. | methylprednisolone 1000 mg |
| | Administer methylprednisolone 1000 mg | IV per day for 3 days; if |
| | IV per day with first dose of tocilizumab | improves, then manage as |
| | and continue methylprednisolone 1000 | above. |
| | mg IV per day for 2 more days; if | |
| | improves, then manage as above. | |

Consider non-sedating, anti-seizure medicines
(e.g., levetiracetam) for seizure prophylaxis.

Additional Safety Management Strategies with Corticosteroids

Administration of corticosteroids and/or tocilizumab at Grade 1 may be considered prophylactic. Supportive care may be provided in all protocols at all CRS and NE severity grades. In one embodiment of a protocol for management of adverse events related to CRS, tocilizumab and/or corticosteroids are administered as follows: Grade 1 CRS: no tocilizumab; no corticosteroids; Grade 2 CRS: tocilizumab (only in case of comorbidities or older age); and/or corticosteroids (only in case of comorbidities or older age); Grade 3 CRS: tocilizumab; and/or corticosteroids; Grade 4 CRS: tocilizumab; and/or corticosteroids. In another embodiment of a protocol for management of adverse events related to CRS, tocilizumab and/or corticosteroids are administered as follows: Grade 1 CRS: tocilizumab (if no improvement after 3 days); and/or corticosteroids (if no improvement after 3 days); Grade 2 CRS: tocilizumab; and/or corticosteroids; Grade 3 CRS: tocilizumab; and/or corticosteroids; Grade 4 CRS: tocilizumab; and/or corticosteroids, high dose.

In one embodiment of a protocol for management of adverse events related to NE, tocilizumab and/or corticosteroids are administered as follows: Grade 1 NE: no tocilizumab; no corticosteroids; Grade 2 NE: no tocilizumab; no corticosteroids; Grade 3 NE: tocilizumab; and/or corticosteroids (only if no improvement to tocilizumab, standard dose); Grade 4 NE: tocilizumab; and/or corticosteroids. In another embodiment of a protocol for management of adverse events related to NE, tocilizumab and/or corticosteroids are administered as follows: Grade 1 NE: no tocilizumab; and/or corticosteroids; Grade 2 NE: tocilizumab; and/or corticosteroids; Grade 3 NE: tocilizumab; and/or corticosteroids, high dose; Grade 4 NE: tocilizumab; and/or corticosteroids, high dose. In one embodiment, corticosteroid treatment is initiated at CRS grade ≥2 and tocilizumab is initiated at CRS grade ≥2. In one embodiment, corticosteroid treatment is initiated at CRS grade ≥1 and tocilizumab is initiated at CRS grade ≥1. In one embodiment, corticosteroid treatment is initiated at NE grade ≥3 and tocilizumab is initiated at CRS grade ≥3. In one embodiment, corticosteroid treatment is initiated at CRS grade ≥1 and tocilizumab is initiated at CRS grade ≥2. In some embodiments, prophylactic use of tocilizumab administered on Day 2 may decrease the rates of Grade ≥3 CRS. The one or more corticosteroids may be administered at any dose and frequency of administration, which may be adjusted to the severity/grade of the adverse event (e.g., CRS and NE). In another embodiment, corticosteroid administration comprises oral or IV dexamethasone 10 mg, 1-4 times per day. Another embodiment, sometimes referred to as “high-dose” corticosteroids, comprises administration of IV methylprednisone 1 g per day alone, or in combination with dexamethasone. In some embodiments, the one or more corticosteroids are administered at doses of 1-2 mg/kg per day. Generally, the dose of corticosteroid administered is dependent upon the specific corticosteroid, as a difference in potency exists between different corticosteroids. It is typically understood that drugs vary in potency, and that doses may therefore vary, in order to obtain equivalent effects. Equivalence in terms of potency for various glucocorticoids and routes of administration, is well known. Information relating to equivalent steroid dosing (in a non-chronotherapeutic manner) may be found in the British National Formulary (BNF) 37. March 1999. The application also provides dosages and administrations of cells prepared by the methods of the application, for example, an infusion bag of CD19-directed genetically modified autologous T cell immunotherapy, comprises a suspension of chimeric antigen receptor (CAR)-positive T cells in approximately 68 mL for infusion. In some embodiments, the CAR T cells are formulated in approximately 40 mL for infusion In some embodiments, the CAR T cell product is formulated in a total volume of 35, 40, 45, 50, 55, 60, 65, 70, 75, 80, 85, 90, 95, 100, 200, 300, 400, 500, 500, 700, 800, 900, 1000 mL. In one aspect, the dosage and administration of cells prepared by the methods of the application, for example, an infusion bag of CD19-directed genetically modified autologous T cell immunotherapy, comprises a suspension of 1×10⁶ CAR-T positive cells in approximately 40 mL. The target dose may be between about 1×10⁶ and about 2×10⁵ CAR-positive viable T cells per kg body weight, with a maximum of 2×10⁸ CAR-positive viable T cells.

In some embodiments, the dosage form comprises a cell suspension for infusion in a single-use, patient-specific infusion bag; the route of administration is intravenous; the entire contents of each single-use, patient-specific bag is infused by gravity or a peristaltic pump over 30 minutes. In one embodiment, the dosing regimen is a single infusion consisting of 2.0×10⁶ anti-CD19 CAR T cells/kg of body weight (±20%), with a maximum dose of 2×10³ anti-CD19 CAR T cells (for subjects ≥100 kg). In some embodiments, the T cells that make up the dose are CD19 CAR-T cells.

In some embodiments, the CD19-directed T cell immunotherapy is KTE-X19, which is prepared as described elsewhere in this application. In one embodiment. KTE-X19 may be used for treatment of MCL, ALL, CLL, SLL, and any other B cell malignancy. In some embodiment, the CD19-directed genetically modified autologous T cell immunotherapy is Axi-cel™ (YESCARTA®, axicabtagene ciloleucel) prepared by one of the methods of the application. Amounts of CAR T cells, dosage regimens, methods of administration, subjects, cancers, that fall within the scope of these methods are described elsewhere in this application, alone or in combination with another chemotherapeutic agent, with or without preconditioning, and to any of the patients described elsewhere in the application.

The following examples are intended to illustrate various aspects of the application. As such, the specific aspects discussed are not to be construed as limitations on the scope of the application. For example, although the Examples below are directed to T cells transduced with an anti-CD19 chimeric antigen receptor (CAR), one skilled in the art would understand that the methods described herein may apply to immune cells transduced with any CAR. It will be apparent to one skilled in the art that various equivalents, changes, and modifications may be made without departing from the scope of application, and it is understood that such equivalent aspects are to be included herein. Further, all references cited in the application are hereby incorporated by reference in their entirety, as if fully set forth herein.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, dictionaries, documents, manuscripts, genomic database sequences, and scientific literature cited herein are hereby incorporated by reference.

Other features and advantages of the disclosure will be apparent from the Examples.

### Example 1

This example describes a phase 2 multicenter study (ZUMA-2) evaluating the efficacy of Brexucabtagene Autoleucel (KTE-X19) in patients with relapsed/refractory mantle cell lymphoma (R/R MCL) who were not previously treated with a Bruton Tyrosine Kinase Inhibitor (BTKi).

This phase 2 study is a multicenter, open-label study evaluating the efficacy of brexu-cel in patients with R/R MCL. The key eligibility criteria for Cohort 3 (NCT04880434) include patients ≥18 years of age with MCL who have received 1 to 5 prior regimens including prior anthracycline-, bendamustine-, or high-dose cytarabine-containing chemotherapy, and an anti-CD20 monoclonal antibody, but have not received prior therapy with a BTKi. History of prior allogeneic stem cell transplant (alloSCT) is allowed if no donor cells are detected on chimerism >100 days after alloSCT. Patients receive conditioning chemotherapy of fludarabine 30 mg/m²/day and cyclophosphamide 500 mg/m²/day, administered on Days −5, −4, and −3, followed by a single infusion of brexu-cel at a target dose of 2×10⁶ anti-CD19 CAR T cells/kg on Day 0. At the discretion of the investigator, bridging therapy with dexamethasone, radiotherapy, specified chemotherapy, or any combination thereof, is recommended for all patients in Cohort 3, particularly those with rapidly progressing disease, clinical deterioration, or high disease burden at screening.

The primary endpoint is ORR as assessed by an independent radiologic review committee per the Lugano classification. Secondary endpoints include safety, duration of response, progression-free survival, overall survival, levels of circulating CAR T cells and cytokines, and change in patient-reported outcomes over time. In the pivotal cohort. EuroQol five-dimensional (EQ-5D) scores were assessed through Month 6 only, whereas for Cohort 3, EQ-5D and the European Organization for Research and Treatment of Cancer Quality of Life Questionnaire (EORTC-QLQ-C30) scores will be assessed over time. Minimal residual disease will be assessed for Cohort 3 via next generation sequencing of ctDNA through Month 24.

Approximately 90 patients will be enrolled in Cohort 3 with a target ORR of 75%, with a hypothesis that the observed ORR will be significantly greater than 57%, the historical control rate based on systematic literature review and meta-analysis. The primary analysis of Cohort 3 will occur after 86 patients have been enrolled and treated with brexu-cel, and have had the opportunity to be assessed for response 6 months after the first objective response or 9 months after brexu-cel infusion, whichever occurs sooner. ZUMA-2 Cohort 3 is currently enrolling patients at 41 sites in the United States, France, Germany, the Netherlands, Spain, and the United Kingdom.

### Example 2

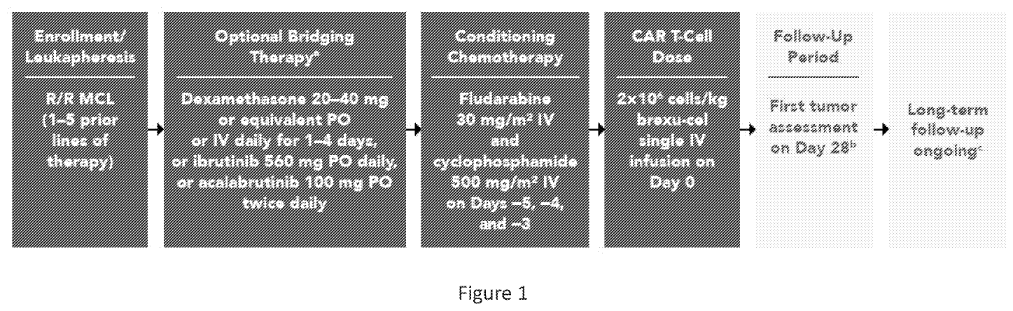

This example describes assessment of durable responses after Brexucabtagene Autoleucel (KTE-X19) in the ZUMA-2 study in relapsed/refractory mantle cell lymphoma (R/R MCL). The study design is shown in FIG. 1. After 35.6 months follow-up in ZUMA-2, brexu-cel demonstrated an objective response rate (ORR; complete response [CR]+partial response [PR]) of 91% (95% CI, 81.8 to 96.7), a CR rate of 68% (95% CI, 55.2 to 78.5), a median duration of response (DOR) of 28.2 months (95% CI, 13.5 to 47.1), and a median overall survival (OS) of 46.6 months (95% CI, 24.9 to not estimable) in all 68 treated patients and not reached in patients with CR. To identify factors associated with long-term response to KTE-X19, patient and product characteristics were evaluated by response status at 24 mo post infusion in ZUMA-2.

Methods: Key ZUMA-2 Eligibility Criteria include: Adults (≥18 years) with R/R MCL who had received 1-5 prior regimens including anthracycline- or bendamustine-containing chemotherapy, anti-CD20 monoclonal antibody, and a BTKi and underwent leukapheresis and conditioning chemotherapy followed by a single infusion of brexu-cel (2×10⁶ anti-CD19 CAR T cells/kg). The primary endpoint is ORR (Objective Response Rate) which refers to (Complete Response (CR)+Partial Response (PR); independent radiology review committee (IRRC) assessed per the Lugano classification. Key secondary endpoints include duration of response (DOR), progression-free survival (PFS), overall survival (OS) and adverse events (AE). Post hoc assessments of patient, disease, pharmacokinetic, and product characteristics are reported by response status at 24 mo (ongoing vs relapsed). Baseline patient and disease characteristics, subsequent therapies, product characteristics, and pharmacologic outcomes were assessed by response status at 24 months after brexu-cel infusion: ongoing responders: patients with ongoing response at their 24-month assessment; relapsed responders: patients with response who relapsed prior to their 24-month assessment; non-responders: patients with no response. DOR was assessed in ongoing responders and relapsed responders. Statistical analyses: time-to-event endpoints were analyzed using the Kaplan-Meier method; all subgroup analyses were descriptive.

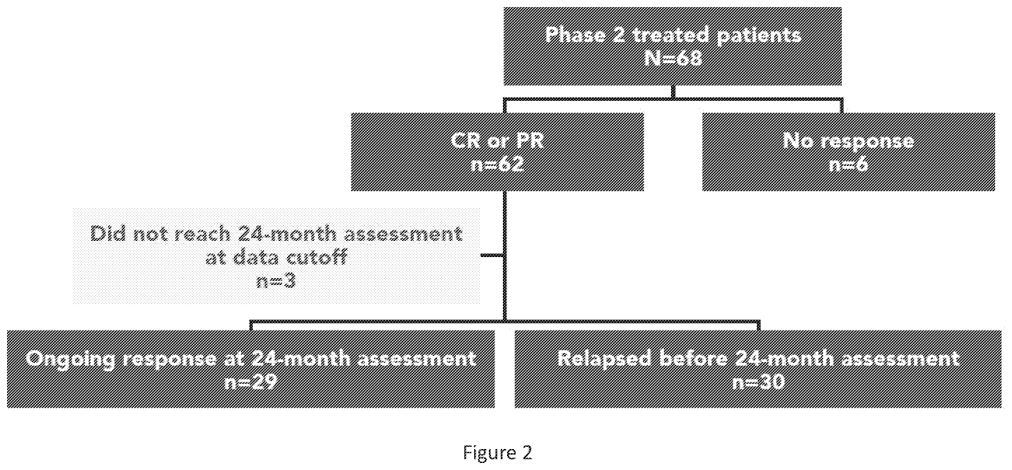

Results: At a median follow-up of 35.6 mo (range, 25.9-56.3), 74 patients were enrolled and leukapheresed, and 68 patients received brexu-cel (FIG. 2).

TABLE 1
Baseline Patient and Disease Characteristics by Response Status at 24 Months

| | | | |
|---|---|---|---|
| Median age (range), years | 65 (50-79) | 65 (38-75) | 66.5 (60-74) |
| Male, n (%) | 26 (90) | 24 (80) | 5 (83) |
| ECOG PS of 0, n (%) | 23 (79) | 17 (57) | 3 (50) |
| Intermediate or high sMIPI, n (%) | 19 (66) | 15 (50) | 4 (67) |
| Ki-67 PI ≥ 30%, n (%) | 19 (66) | 18 (60) | 4 (67) |
| Median no. of prior therapies, n | 3 (2-5) | 3 (2-5) | 3 (1-5) |
| (range) | | | |
| Prior platinum, n (%) | 3 (10) | 12 (40) | 0 |
| Prior anthracycline, n (%) | 22 (76) | 22 (73) | 4 (67) |
| Prior bendamustine, n (%) | 13 (45) | 16 (53) | 6 (100) |
| Prior lenalidomide, n (%) | 7 (24) | 10 (33) | 2 (33) |
| Prior proteasome inhibitor, n (%) | 12 (41) | 11 (37) | 1 (17) |
| Prior autologous SCT, n (%) | 14 (48) | 11 (37) | 2 (33) |
| Prior BTKi therapy, n (%) | 29 (100) | 30 (100) | 6 (100) |
| Ibrutinib | 27 (93) | 24 (80) | 4 (67) |
| Acalabrutinib | 8 (28) | 6 (20) | 2 (33) |
| Both | 6 (21) | 0 | 0 |

Relapsed or refractory disease, n (%)

| | | | |
|---|---|---|---|
| Relapse after autologous SCT | 14 (48) | 11 (37) | 2 (33) |
| Refractory to last MCL therapy | 10 (34) | 13 (43) | 3 (50) |
| Relapse after last MCL therapy | 5 (17) | 6 (20) | 1 (17) |
| CD19 positive IHC by central lab, | 22 (76) | 20 (67) | 3 (50) |
| n (%) | | | |

Tumor burden (SPD) by central read (mm2)

| | | | |
|---|---|---|---|
| n | 28 | 28 | 4 |
| Median (range) | 935.1 (260-6133) | 4233.6 (386-14390) | 553.1 (293-16878) |
| Positive bone marrow assessment | 16 (55) | 16 (53) | 5 (83) |
| at baseline, n (%) | | | |
| Elevated LDH levels (ULN to ≥1.5 | 13 (45) | 11 (37) | 2 (33) |
| ULN), n (%) | | | |
| Received bridging therapy, n (%)a | 6 (21) | 16 (53) | 3 (50) |

aBridging therapy was received after leukapheresis and prior to conditioning chemotherapy in ZUMA-2. A smaller proportion of ongoing responders compared with relapsed responders received bridging therapy and had Eastern Cooperative Oncology Group performance status (ECOG PS) scores of 1, with the median tumor burden (sum of the products of diameters) at baseline being ~4 times smaller in ongoing responders compared with relapsed responders (Table 1). The median number of prior therapies was 3 in both subgroups with a smaller proportion of ongoing responders compared with relapsed responders receiving prior platinum therapy (Table 1).

TABLE 2
Last Prior Therapies by Response Status at 24 Months

| | | |
|---|---|---|
| Ongoing | Relapsed | Non- |
| Responders | Responders | responders |
| (n = 29) | (n = 30) | (n = 6) |

Last prior therapy, n (%)

| | | | |
|---|---|---|---|
| Acalabrutinib | 4 (14) | 4 (13) | 1 (17) |
| Bendamustine | 2 (7) | 3 (10) | 1 (17) |
| Cisplatin | 0 | 1 (3) | 0 |
| Cyclophosphamide | 0 | 1 (3) | 0 |
| Cytarabine | 0 | 1 (3) | 0 |
| Gemcitabine | 1 (3) | 0 | 0 |
| Ibrutinib | 19 (66) | 13 (43) | 2 (33) |
| Lenalidomide | 1 (3) | 4 (13) | 1 (17) |
| Protein kinase inhibitors | 1 (3) | 0 | 0 |
| Rituximab | 1 (3) | 1 (3) | 1 (17) |
| Venetoclax | 0 | 2 (7) | 0 |
| Median (range) time from | 63.0 (26-748) | 64.5 (22-443) | 136 (29-642) |
| last prior therapy to | | | |
| brexu-cel infusion (days) | | | |

Ibrutinib was more commonly the last prior therapy in ongoing responders versus relapsed responders while a similar proportion received acalabrutinib as their last prior therapy (Table 2). Median time from last prior therapy to brexu-cel infusion was similar among ongoing and relapsed responders but was more than twice as long in non-responders, though the small sample size may have contributed to this difference.
62 pts had achieved a CR or partial response; 3 did not reach the 24-mo assessment visit and were excluded from this analysis. Of the 59 evaluable pts with response, 29 (47%) had ongoing responses at 24 mo (ongoing responders), and 30 (48%) had relapsed prior to 24 mo (relapsed responders). 6 patients did not respond (non-responders). At baseline, the median age was 65 years and median number of prior therapies was 3 in both subgroups. Among ongoing vs relapsed responders, 66% vs 43% had ibrutinib and 14% vs 13% bad acalabrutinib as last prior therapy, with a median (range) time from last prior therapy of 63 (26-748) vs 64.5 mo (22-443). Compared with relapsed responders, a smaller proportion of ongoing responders received bridging therapy (53% vs 21%, respectively) and prior platinum therapy (40% vs 10%); while similar proportions received prior bendamustine therapy (53% vs 45%), prior proteosome inhibitor therapy (37% and 41%), and prior autologous stem cell transplant (37% vs 48%).

At baseline, a greater proportion of ongoing responders had an ECOG score of 0 compared to relapsed responders (79% vs 57%, respectively), and the median (range) tumor burden (SPD) was 935.1 (260-6133) in ongoing responders and 4233.6 (386-14390) in relapsed responders. The incidence of high-risk features was similar between ongoing responders and relapsed responders, with 66% and 60% having a baseline Ki-67 proliferation index score of ≥30%, 10% and 10% having TP53 mutations, 45% and 37% having elevated lactose dehydrogenase levels (≥ULN to ≤1.5 ULN), and 10% and 13% having high-risk Simplified Mantle Cell Lymphoma International Prognostic Index scores (>6), respectively.

TABLE 3
Duration of Response (DOR) using Central Read per Cheson 2014 (Cohort
1: KTE-X19) (mITT Analysis Set: Subjects with Complete Response)

| | Responders | Responders | |
|---|---|---|---|
| | on/after | Relapsed before | |
| | Month 24 Visit | Month 24 Visit | Overall |
| | (N = 28) | (N = 15) | (N = 43) |
| DOR^{a} | | | |
| Subjects with a complete response | 28 | 15 | 43 |
| Event, n (%) | 5 (18) | 12 (80) | 17 (40) |
| Censored, n (%) | 23 (82) | 3 (20) | 26 (60) |
| KM median (95% CI) DOR time | NR (46.7, NE) | 8.3 (5.0, 13.6) | 46.7 (24.4, NE) |
| (months) | | | |
| Min, max DOR (months) | 20.5⁺, 53.0⁺ | 0.0⁺, 17.1 | 0.0⁺, 53.0⁺ |
| Type of event | | | |
| Disease progression, n (%) | 2 (7) | 12 (80) | 14 (33) |
| Death, n (%) | 3 (11) | 0 (0) | 3 (7) |
| Censoring reason | | | |
| Response ongoing, n (%) | 22 (79) | 0 (0) | 22 (51) |
| SCT, n (%) | 0 (0) | 2 (13) | 2 (5) |
| Started non-SCT new anticancer | 1 (4) | 1 (7) | 2 (5) |
| therapy, n (%) | | | |
| Event-free rate (% [95% CI]) by KM | | | |
| estimation at | | | |
| 3 months | 100 (100, 100) | 100 (100, 100) | 100 (100, 100) |
| 6 months | 100 (100, 100) | 75.0 (40.8, 91.2) | 92.5 (78.5, 97.5) |
| 9 months | 100 (100, 100) | 33.3 (10.3, 58.8) | 80.0 (64.0, 89.5) |
| 12 months | 100 (100, 100) | 25.0 (6.0, 50.5) | 77.5 (61.2, 87.6) |
| 15 months | 100 (100, 100) | 8.3 (0.5, 31.1) | 72.5 (55.9, 83.7) |
| 18 months | 100 (100, 100) | 0.0 (NE, NE) | 70.0 (53.3, 81.7) |
| 21 months | 100 (100, 100) | 0.0 (NE, NE) | 70.0 (53.3, 81.7) |
| 24 months | 100 (100, 100) | 0.0 (NE, NE) | 70.0 (53.3, 81.7) |
| 27 months | 90.0 (65.6, 97.4) | 0.0 (NE, NE) | 63.0 (45.2, 76.4) |
| 30 months | 90.0 (65.6, 97.4) | 0.0 (NE, NE) | 63.0 (45.2, 76.4) |
| 33 months | 90.0 (65.6, 97.4) | 0.0 (NE, NE) | 63.0 (45.2, 76.4) |
| 36 months | 90.0 (65.6, 97.4) | 0.0 (NE, NE) | 63.0 (45.2, 76.4) |
| 39 months | 81.8 (52.0, 94.0) | 0.0 (NE, NE) | 57.3 (37.7, 72.7) |
| 42 months | 81.8 (52.0, 94.0) | 0.0 (NE, NE) | 57.3 (37.7, 72.7) |
| 45 months | 81.8 (52.0, 94.0) | 0.0 (NE, NE) | 57.3 (37.7, 72.7) |
| 48 months | 51.1 (14.5, 79.2) | 0.0 (NE, NE) | 35.8 (12.1, 60.6) |
| 51 months | 51.1 (14.5, 79.2) | 0.0 (NE, NE) | 35.8 (12.1, 60.6) |
| Median (95% CI) follow-up time | 30.2 (26.8, 44.9) | NE (1.9, NE) | 29.8 (23.2, 44.8) |
| (months) for DOR (reverse KM | | | |
| approach) | | | |

Abbreviations: CI, confidence interval; DOR, duration of response; KM, Kaplan-Meier; mITT, modified intent-to-treat; NE, not estimable; NR, not reached; SCT, stem cell transplant; ‘⁺’ indicates censored record.
Note:
Percentages are based on the number of subjects treated with KTE-X19 and had an complete response.
^{a}DOR is defined as the time from the first objective response to disease progression or death prior to new anti-cancer therapy (including SCT).
Median Follow-up Time for DOR is estimated using reverse KM approach.
3 subjects who remain responses prior to DCO and had the last available assessment before Month 24 visit are excluded from this table.

The median (range) DOR in ongoing responders with CR (n=28) was not reached (46.7-not estimable) and was 8.3 mo (5-13.6) in relapsed responders with CR (n=15, Table 3). The median time to initial response for ongoing versus relapsed responders was 1 month (range, 0.9-3.1; n=29) vs 1 month (range, 0.8-1.7; n=30). The median time to complete response for ongoing versus relapsed responders was 3 months (range, 0.9-35.1; n=28) vs 3 months (range, 0.8-9.0; n=15). Median time for conversion from SD or PR to CR for ongoing versus relapsed responders was 2.3 months (range, 1.8-34.1; n=16) vs 2.4 months (range, 2.0-8.1; n=8).

The median (95% CI) DOR in ongoing responders with CR who had high baseline LDH levels (n=12) was 47.1 months (24.8-not estimable) and was 8.3 months (4.7-NE) in relapsed responders with CR who had high baseline LDH levels (n=5).

TABLE 4
Duration of Response (DOR) using Central Read per Cheson 2014 (Cohort 1; KTE-X19)
(mITT Analysis Set: Subjects with Complete Response and High Baseline LDH Level)

| | Responders | Responders | |
|---|---|---|---|
| | on/after | Relapsed before | |
| | Month 24 Visit | Month 24 Visit | Overall |
| | (N = 12) | (N = 5) | (N = 17) |
| DOR^{a} | | | |
| Subjects with a complete response | 12 | 5 | 17 |
| Event, n (%) | 3 (25) | 3 (60) | 6 (35) |
| Censored, n (%) | 9 (75) | 2 (40) | 11 (65) |
| KM median (95% CI) DOR time | 47.1 (24.8, NE) | 8.3 (4.7, NE) | 47.1 (14.4, NE) |
| (months) | | | |
| Min, max DOR (months) | 20.5⁺, 47.8⁺ | 0.0⁺, 14.4 | 0.0⁺, 47.8⁺ |
| Type of event | | | |
| Disease progression, n (%) | 2 (17) | 3 (60) | 5 (29) |
| Death, n (%) | 1 (8) | 0 (0) | 1 (6) |
| Censoring reason | | | |
| Response ongoing, n (%) | 8 (67) | 0 (0) | 8 (47) |
| SCT, n (%) | 0 (0) | 1 (20) | 1 (6) |
| Started non-SCT new anticancer | 1 (8) | 1 (20) | 2 (12) |
| therapy, n (%) | | | |
| Event-free rate (% [95% CI]) by KM | | | |
| estimation at | | | |
| 3 months | 100 (100, 100) | 100 (100, 100) | 100 (100, 100) |
| 6 months | 100 (100, 100) | 66.7 (5.4, 94.5) | 93.3 (61.3, 99.0) |
| 9 months | 100 (100, 100) | 33.3 (0.9, 77.4) | 86.7 (56.4, 96.5) |
| 12 months | 100 (100, 100) | 33.3 (0.9, 77.4) | 86.7 (56.4, 96.5) |
| 15 months | 100 (100, 100) | 0.0 (NE, NE) | 80.0 (50.0, 93.1) |
| 18 months | 100 (100, 100) | 0.0 (NE, NE) | 80.0 (50.0, 93.1) |
| 21 months | 100 (100, 100) | 0.0 (NE, NE) | 80.0 (50.0, 93.1) |
| 24 months | 100 (100, 100) | 0.0 (NE, NE) | 80.0 (50.0, 93.1) |
| 27 months | 87.5 (38.7, 98.1) | 0.0 (NE, NE) | 70.0 (37.2, 87.9) |
| 30 months | 87.5 (38.7, 98.1) | 0.0 (NE, NE) | 70.0 (37.2, 87.9) |
| 33 months | 87.5 (38.7, 98.1) | 0.0 (NE, NE) | 70.0 (37.2, 87.9) |
| 36 months | 87.5 (38.7, 98.1) | 0.0 (NE, NE) | 70.0 (37.2, 87.9) |
| 39 months | 65.6 (15.7, 90.9) | 0.0 (NE, NE) | 52.5 (16.0, 79.7) |
| 42 months | 65.6 (15.7, 90.9) | 0.0 (NE, NE) | 52.5 (16.0, 79.7) |
| 45 months | 65.6 (15.7, 90.9) | 0.0 (NE, NE) | 52.5 (16.0, 79.7) |
| Median (95% CI) follow-up time | | | |
| (months) | | | |
| for DOR (reverse KM approach) | 28.0 (22.8, NE) | NE (0.0, NE) | 28.0 (22.8, 47.2) |

Abbreviations: CI, confidence interval; DOR, duration of response; KM, Kaplan-Meier; LDH, lactose dehydrogenase; mITT, modified intent-to-treat; NE, not estimable; NR, not reached; SCT, stem cell transplant; ‘⁺’ indicates censored record.
Note:
Percentages are based on the number of subjects treated with KTE-X19 and had a complete response and baseline LDH level >= ULN.
^{a}DOR is defined as the time from the first objective response to disease progression or death prior to new anti-cancer therapy (including SCT).
Median Follow-up Time for DOR is estimated using reverse KM approach.
3 subjects who remain responses prior to DCO and had the last available assessment before Month 24 visit are excluded from this table.

TABLE 5
Duration of Response (DOR) using Central Read per Cheson 2014 (Cohort 1: KTE-X19)
(mITT Analysis Set: Subjects with Objective Response and High Baseline LDH Level)

| | Responders | Responders | |
|---|---|---|---|
| | on/after | Relapsed before | |
| | Month 24 Visit | Month 24 Visit | Overall |
| | (N = 13) | (N = 11) | (N = 24) |
| DOR^{a} | | | |
| Subjects with an objective | 13 | 11 | 24 |
| response | | | |
| Event, n (%) | 4 (31) | 9 (82) | 13 (54) |
| Censored, n (%) | 9 (69) | 2 (18) | 11 (46) |
| KM median (95% CI) DOR time | 47.1 (24.8, NE) | 3.6 (1.0, 13.5) | 28.2 (8.3, 47.1) |
| (months) | | | |
| Min, max DOR (months) | 20.5⁺, 47.8⁺ | 0.0⁺, 14.4 | 0.0⁺, 47.8⁺ |
| Type of event | | | |
| Disease progression, n (%) | 3 (23) | 8 (73) | 11 (46) |
| Death, n (%) | 1 (8) | 1 (9) | 2 (8) |
| Censoring reason | | | |
| Response ongoing, n (%) | 8 (62) | 0 (0) | 8 (33) |
| SCT, n (%) | 0 (0) | 1 (9) | 1 (4) |
| Started non-SCT new anticancer | 1 (8) | 1 (9) | 2 (8) |
| therapy, n (%) | | | |
| Event-free rate (% [95% CI]) by KM | | | |
| estimation at | | | |
| 3 months | 100 (100, 100) | 60.0 (25.3, 82.7) | 82.6 (60.1, 93.1) |
| 6 months | 100 (100, 100) | 36.0 (9.0, 64.8) | 73.4 (50.1, 87.1) |
| 9 months | 100 (100, 100) | 24.0 (3.8, 53.7) | 68.8 (45.5, 83.8) |
| 12 months | 100 (100, 100) | 24.0 (3.8, 53.7) | 68.8 (45.5, 83.8) |
| 15 months | 100 (100, 100) | 0.0 (NE, NE) | 59.7 (36.7, 76.6) |
| 18 months | 100 (100, 100) | 0.0 (NE, NE) | 59.7 (36.7, 76.6) |
| 21 months | 100 (100, 100) | 0.0 (NE, NE) | 59.7 (36.7, 76.6) |
| 24 months | 100 (100, 100) | 0.0 (NE, NE) | 59.7 (36.7, 76.6) |
| 27 months | 88.9 (43.3, 98.4) | 0.0 (NE, NE) | 53.0 (29.6, 71.8) |
| 30 months | 74.1 (28.9, 93.0) | 0.0 (NE, NE) | 44.2 (20.3, 65.8) |
| 33 months | 74.1 (28.9, 93.0) | 0.0 (NE, NE) | 44.2 (20.3, 65.8) |
| 36 months | 74.1 (28.9, 93.0) | 0.0 (NE, NE) | 44.2 (20.3, 65.8) |
| 39 months | 55.6 (14.1, 83.8) | 0.0 (NE, NE) | 33.1 (10.5, 58.2) |
| 42 months | 55.6 (14.1, 83.8) | 0.0 (NE, NE) | 33.1 (10.5, 58.2) |
| 45 months | 55.6 (14.1, 83.8) | 0.0 (NE, NE) | 33.1 (10.5, 58.2) |
| Median (95% CI) follow-up time | | | |
| (months) | | | |
| for DOR (reverse KM approach) | 30.2 (23.1, NE) | NE (1.9, NE) | 28.0 (22.8, NE) |

Abbreviations: CI, confidence interval; DOR, duration of response; KM, Kaplan-Meier; LDH, lactose dehydrogenase; mITT, modified intent-to-treat; NE, not estimable; NR, not reached; SCT, stem cell transplant; ‘⁺’ indicates censored record.
Note:
Percentages are based on the number of subjects treated with KTE-X19 and had an objective response and baseline LDH level >= ULN.
^{a}DOR is defined as the time from the first objective response to disease progression or death prior to new anti-cancer therapy (including SCT).
Median Follow-up Time for DOR is estimated using reverse KM approach.
3 subjects who remain responses prior to DCO and had the last available assessment before Month 24 visit are excluded from this table.

TABLE 6
Subsequent Therapies by Response Status at 24 Months

| | Ongoing | Relapsed | Non- |
|---|---|---|---|
| WHO-DD Preferred | Responders | Responders | responders |
| Name, n (%) | (n = 29) | (n = 30) | (n = 6) |
| Patients who had subsequent | 1 (3) | 20 (67) | 3 (50) |
| anticancer therapy^{a} | | | |
| Acalabrutinib | 1 (3) | 2 (7) | 1 (17) |
| Bortezomib | 0 | 4 (13) | 1 (17) |
| Cytarabine | 0 | 3 (10) | 1 (17) |
| Ibrutinib | 0 | 5 (17) | 1 (17) |
| Lenalidomide | 0 | 6 (20) | 1 (17) |
| Obinutuzumab | 0 | 2 (7) | 1 (17) |
| Radiotherapy | 1 (3) | 7 (23) | 1 (17) |
| Rituximab | 0 | 7 (23) | 1 (17) |
| Venetoclax | 1 (3) | 6 (20) | 1 (17) |
| Bendamustine | 0 | 2 (7) | 0 |
| Cyclophosphamide | 0 | 5 (17) | 0 |
| Dexamethasone | 0 | 7 (23) | 0 |
| Fludarabine | 0 | 3 (10) | 0 |
| Fludarabine phosphate | 0 | 2 (7) | 0 |
| Melphalan | 0 | 3 (10) | 0 |
| Methotrexate | 0 | 3 (10) | 0 |

Of the relapsed responders. 67% had received subsequent anticancer therapy by data cutoff, the most common of which were radiotherapy (23%), dexamethasone (23%), rituximab (23%), venetoclax (20%), and lenalidomide (20%; patients could have received multiple subsequent therapies and multiple lines of subsequent therapy).

TABLE 7
Summary of Peak and AUC of Anti-CD19 CAR T Cells in Blood
(cells/μL) (Cohort 1: KTE-X19) (mITT Analysis Set)

| | | | |
|---|---|---|---|
| Responders on/after Month | Responders Relapsed | | |
| 24 | before | | |
| Visit | Month 24 Visit | Non-Responders | Overall |
| (N = 29) | (N = 30) | (N = 6) | (N = 65) |

Area Under Curve (From Day 0 To Day 28)

| | | | | |
|---|---|---|---|---|
| n | 29 | 30 | 6 | 65 |
| Mean (STDEV) | 2938.18 | 3296.61 | 239.88 | 2854.54 |
| | (3993.53) | (5775.57) | (431.10) | (4779.39) |
| Median (Q1, Q3) | 1486.96 | 688.21 | 24.66 | 1089.11 |
| | (426.56, 3049.87) | (206.27, 4599.78) | (5.21, 293.86) | (242.52, 2960.77) |
| Min, Max | 3.83, | 19.03, | 1.81, | 1.81, |
| | 1.67E+04 | 2.72E+04 | 1089.11 | 2.72E+04 |

Peak

| | | | | |
|---|---|---|---|---|
| n | 29 | 30 | 6 | 65 |
| Mean (STDEV) | 297.68 | 326.57 | 21.70 | 285.54 |
| | (516.42) | (582.28) | (37.39) | (527.07) |
| Median (Q1, Q3) | 102.43 | 59.86 | 5.87 | 77.60 |
| | (33.33, 264.33) | (17.63, 297.80) | (0.33, 22.02) | (17.63, 264.33) |
| Min, Max | 0.27, | 1.60, | 0.16, | 0.16, |
| | 2241.62 | 2589.47 | 95.92 | 2589.47 |

Abbreviations: n—number of subjects, Max—maximum, Min—minimum, Q—quartile, STDEV—standard deviation
3 subjects who remain responses prior to DCO and had the last available assessment before Month 24 visit are excluded from this table.
Area under curve (AUC) is defined as the area under curve in a plot of number of CAR T cells against scheduled visit from Day 0 to Day 28.
Peak is defined as the maximum number of CAR T measured post infusion.

Median [range] peak (102.4 [0.3-2241.6] vs 59.9 [1.6-2589.5]) and area under the curve (1487 [3.8-0.0002] vs 688.2 [19-0.0003]) CAR T-cell levels were ˜2× higher in ongoing responders than in relapsed responders, respectively (Table 7). A modest increase in the median [range] total number of infused CCR7+ cells was observed in ongoing vs relapsed responders (119.8 [37-249.9] vs 89.1 [6.1-353.4]), suggesting a need for further investigation into the role of continuous memory T-cell differentiation in achieving durable responses.

TABLE 8
Summary of Product Characteristics
by Response Status at 24 Months

| | Ongoing | Relapsed | Non- |
|---|---|---|---|
| | Responders | Responders | responders |
| Median (range) | (n = 29) | (n = 30) | (n = 6) |
| CD4/CD8 Ratio | 0.86 | 0.62 | 0.4 |
| | (0.27-2.06) | (0.04-3.73) | (0.3-0.7) |
| Total Number of CCR7+ | 119.8 | 89.1 | 88.2 |
| T Cells Infused (10⁶) | (37.0-249.9) | (6.1-353.4) | (39.9-150.3) |

Product characteristics were largely similar among ongoing and relapsed responders with a modest increase in the median total number of infused CCR7+ T cells observed in ongoing vs relapsed responders (Table 8)

TABLE 9A
Peripheral Blood T Cell Phenotype at Day 7
by Response Status - CD8+CD27−CD28+, Ongoing
(n = 21), Other (n = 32)

| | |
|---|---|
| Other | 28.5056 |
| Other | 24.5752 |
| Other | 24.1402 |
| Other | 17.7935 |
| Other | 16.7257 |
| Other | 15.7765 |
| Other | 12.7187 |
| Other | 11.6975 |
| Other | 10.6953 |
| Other | 9.1228 |
| Other | 6.4495 |
| Other | 6.3839 |
| Other | 5.9232 |
| Other | 5.6394 |
| Other | 5.1281 |
| Other | 4.7302 |
| Other | 4.7175 |
| Other | 4.5996 |
| Other | 3.8083 |
| Other | 3.5688 |
| Other | 3.1562 |
| Other | 3.1228 |
| Other | 2.2857 |
| Other | 2.2243 |
| Other | 1.8468 |
| Other | 1.6059 |
| Other | 1.5957 |
| Other | 1.4278 |
| Other | 1.4203 |
| Other | 1.1425 |
| Other | 1.071 |
| Other | 0.2534 |
| Ongoing | 11.4851 |
| Ongoing | 6.2941 |
| Ongoing | 5.1126 |
| Ongoing | 4.7631 |
| Ongoing | 3.7548 |
| Ongoing | 3.6998 |
| Ongoing | 2.5356 |
| Ongoing | 2.2805 |
| Ongoing | 2.222 |
| Ongoing | 2.1331 |
| Ongoing | 1.8719 |
| Ongoing | 1.6867 |
| Ongoing | 1.5396 |
| Ongoing | 1.435 |
| Ongoing | 0.9271 |
| Ongoing | 0.8997 |
| Ongoing | 0.8733 |
| Ongoing | 0.8318 |
| Ongoing | 0.5898 |
| Ongoing | 0.5654 |
| Ongoing | 0.434 |

TABLE 9B
Peripheral Blood T Cell Phenotype at Day 7
by Response Status - CD8+ CCR7−
CD45RA+ CD27− CD28+, Ongoing (n = 21), Other (n = 32)

| | |
|---|---|
| Other | 16.9166 |
| Other | 38.5378 |
| Other | 15.8358 |
| Other | 14.3717 |
| Other | 19.5668 |
| Other | 15.2561 |
| Other | 9.6467 |
| Other | 4.4728 |
| Other | 9.5349 |
| Other | 7.5791 |
| Other | 2.984 |
| Other | 17.398 |
| Other | 5.4453 |
| Other | 4.444 |
| Other | 4.2978 |
| Other | 1.5938 |
| Other | 3.3624 |
| Other | 1.6179 |
| Other | 3.5814 |
| Other | 3.4151 |
| Other | 1.9248 |
| Other | 1.3394 |
| Other | 2.6589 |
| Other | 1.8797 |
| Other | 2.4221 |
| Other | 1.7701 |
| Other | 0 |
| Other | 0.7648 |
| Other | 2.0531 |
| Other | 1.2586 |
| Other | 0.5908 |
| Other | 0.1751 |
| Ongoing | 8.5695 |
| Ongoing | 11.1365 |
| Ongoing | 5.6489 |
| Ongoing | 1.2954 |
| Ongoing | 2.8111 |
| Ongoing | 2.1458 |
| Ongoing | 1.516 |
| Ongoing | 0.9417 |
| Ongoing | 3.1841 |
| Ongoing | 1.6962 |
| Ongoing | 4.5143 |
| Ongoing | 1.0812 |
| Ongoing | 1.1472 |
| Ongoing | 0.752 |
| Ongoing | 0.6708 |
| Ongoing | 0.979 |
| Ongoing | 0.534 |
| Ongoing | 0.3712 |
| Ongoing | 0.7025 |
| Ongoing | 0.5762 |
| Ongoing | 0.3295 |

TABLE 9C
Peripheral Blood T Cell Phenotype at Day 7 by
Response Status - CD4+ CD27+ CD28−, Ongoing
(n = 21), Other (n = 32)

| | |
|---|---|
| Other | 0.1844 |
| Other | 0.1336 |
| Other | 0.2542 |
| Other | 0.058 |
| Other | 0.2284 |
| Other | 0.156 |
| Other | 0.0804 |
| Other | 3.9815 |
| Other | 0.1872 |
| Other | 0.3955 |
| Other | 0.5679 |
| Other | 1.7191 |
| Other | 3.7187 |
| Other | 4.0962 |
| Other | 0.3447 |
| Other | 1.0355 |
| Other | 0.774 |
| Other | 0.3681 |
| Other | 0.2859 |
| Other | 1.18 |
| Other | 2.57 |
| Other | 4.3683 |
| Other | 7.8571 |
| Other | 2.0031 |
| Other | 4.578 |
| Other | 0.7202 |
| Other | 1.1834 |
| Other | 6.1969 |
| Other | 0.9398 |
| Other | 0.7878 |
| Other | 1.0253 |
| Other | 7.4889 |
| Ongoing | 0.2875 |
| Ongoing | 1.6149 |
| Ongoing | 0.8739 |
| Ongoing | 0.2078 |
| Ongoing | 1.8134 |
| Ongoing | 4.1463 |
| Ongoing | 1.7847 |
| Ongoing | 7.1879 |
| Ongoing | 0.2874 |
| Ongoing | 1.5569 |
| Ongoing | 5.8214 |
| Ongoing | 0.7803 |
| Ongoing | 1.405 |
| Ongoing | 1.0225 |
| Ongoing | 3.2091 |
| Ongoing | 0.5165 |
| Ongoing | 13.123 |
| Ongoing | 7.7951 |
| Ongoing | 4.2367 |
| Ongoing | 31.7742 |
| Ongoing | 11.5187 |

TABLE 9D
Peripheral Blood T Cell Phenotype at Day 7 by Response
Status - CD8+CCR7+CD45RA−PD1+, Ongoing
(n = 21), Other (n = 32)

| | |
|---|---|
| Other | 40.519 |
| Other | 86.8642 |
| Other | 15.3846 |
| Other | 33.913 |
| Other | 37.0812 |
| Other | 0 |
| Other | 52.0879 |
| Other | 89.2857 |
| Other | 36.5508 |
| Other | 34.3939 |
| Other | 67.2566 |
| Other | 74.1488 |
| Other | 58.6207 |
| Other | 37.5998 |
| Other | 75.2577 |
| Other | 38.8942 |
| Other | 43.5054 |
| Other | 36.7063 |
| Other | 37.4563 |
| Other | 60.7277 |
| Other | 71.2644 |
| Other | 69.0875 |
| Other | 84.1758 |
| Other | 78.4314 |
| Other | 30.303 |
| Other | 50 |
| Other | 83.3333 |
| Other | 51.0013 |
| Other | 77.1242 |
| Other | 22.2222 |
| Other | 45.7364 |
| Other | 46.8208 |
| Ongoing | 56.1453 |
| Ongoing | 64.4258 |
| Ongoing | 32.973 |
| Ongoing | 19.1617 |
| Ongoing | 57.5758 |
| Ongoing | 37.3354 |
| Ongoing | 54.6326 |
| Ongoing | 56.2423 |
| Ongoing | 86.911 |
| Ongoing | 53.5714 |
| Ongoing | 93.3032 |
| Ongoing | 61.2691 |
| Ongoing | 92.8571 |
| Ongoing | 54.5455 |
| Ongoing | 90.3226 |
| Ongoing | 95.2854 |
| Ongoing | 57.4132 |
| Ongoing | 58.0357 |
| Ongoing | 44.7826 |
| Ongoing | 83.2041 |
| Ongoing | 87.5 |

Peripheral Blood T cells of relapsed and non-responding patients exhibit a more prominent CD8+CD27−CD28+ effector memory phenotype compared to patients with ongoing response. Ongoing responders are enriched with peripheral CD4 T cells that maintain juvenile CD27+ expression and activated CD8 effector memory T cells.

After ˜3-years median follow-up, brexu-cel continues to demonstrate durable responses with 47% of responders still in ongoing response at 24 mo post infusion. Ongoing responses were observed in patients with high-risk disease characteristics suggesting that brexu-cel has the potential to produce durable responses in patients with R/R MCL who would typically have a poor prognosis. Ibrutinib was more commonly the last prior therapy in ongoing versus relapsed responders. In summary, ongoing responders had lower ECOG PS scores and lower tumor burden, as well as less frequent use of prior platinum therapy or bridging therapy and less intense regimens for previous relapses compared with relapsed responders, suggesting the potential for greater benefit with brexu-cel if given in earlier course of disease. Median peak and AUC CAR T-cell levels were ˜2× higher in ongoing responders than in relapsed responders, suggesting that the degree of CAR T-cell expansion may predict durability of response. A modest increase in the median total number of infused CCR7+ cells and maintenance of CD27+ peripheral T cells observed in ongoing vs relapsed responders may suggest a potential role of continuous memory T-cell differentiation in achieving durable responses.

### Example 3

This example describes a phase 2, open-label, multicenter, basket study (ZUMA-25) evaluating the safety and efficacy of Brexucabtagene Autoleucel in adults with rare B-cell malignancies, including Waldenstrom Macroglobulinemia, Richter Transformation, Burkitt Lymphoma, and Hairy Cell Leukemia.

The primary objective of this study is to evaluate the efficacy of brexucabtagene autoleucel in four rare B-cell malignancies. This study uses a basket study design with separate, indication-specific substudies, to investigate relapsed/refractory Waldenstrom macroglobulinemia (r/r WM), relapsed/refractory Richter transformation (r/r RT), relapsed/refractory Burkitt lymphoma (r/r BL), and relapsed/refractory hairy cell leukemia (r/r HCL).

Substudy A: The primary objective of this substudy is to evaluate the efficacy of brexucabtagene autoleucel in participants with r/r WM by determining the combined rate of complete response (CR) and very good partial response (VGPR) by central assessment. Participants received fludarabine 30 mg/m\hat{ }2/day and cyclophosphamide 500 mg/m\hat{ }2/day lymphodepletion chemotherapy for 3 consecutive days, from Day −5 to Day −3 followed by 2 rest days (Day −2 and Day −1), followed by a single infusion of brexucabtagene autoleucel at target dose of 2×10\hat{ }6 anti-CD19 chimeric antigen receptor (CAR) T cells/kg or 1×10\hat{ }6 anti-CD19 CAR T cells/kg or a flat dose of 2×10⁸ or 1×10⁸, respectively, anti-CD19 CAR T cells in subjects >100 kg.

Substudy B: The primary objective of this substudy is to evaluate the efficacy of brexucabtagene autoleucel on diffuse large B-cell lymphoma-Richter transformation (DLBCL-RT) in participants with r/r RT, by determining the objective response rate (ORR) by central assessment. Participants received fludarabine 30 mg/m\hat{ }2/day and cyclophosphamide 500 mg/m\hat{ }2/day lymphodepletion chemotherapy for 3 consecutive days, from Day −5 to Day −3 followed by 2 rest days (Day −2 and Day −1), followed by a single infusion of brexucabtagene autoleucel at target dose of 2×10\hat{ }6 anti-CD19 CAR T cells/kg or 1×10\hat{ }6 anti-CD19 CAR T cells/kg or a flat dose of 2×10⁸ or 1×10⁸, respectively, anti-CD19 CAR T cells in subjects >100 kg.

Substudy C: The primary objective of this substudy is to evaluate the efficacy of brexucabtagene autoleucel in participants with r/r BL, by determining the ORR by central assessment. Participants received fludarabine 30 mg/m\hat{ }2/day and cyclophosphamide 500 mg/m\hat{ }2/day lymphodepletion chemotherapy for 3 consecutive days, from Day −5 to Day −3 followed by 2 rest days (Day −2 and Day −1), followed by a single infusion of brexucabtagene autoleucel at a target dose of 2×10\hat{ }6 anti-CD19 CAR T cells/kg or 1×10\hat{ }6 anti-CD19 CAR T cells/kg or a flat dose of 2×10⁸ or 1×10⁸, respectively, anti-CD19 CAR T cells in subjects >100 kg.

Substudy D: The primary objective of this substudy is to evaluate the efficacy of brexucabtagene autoleucel in participants with r/r HCL by determining the ORR by central assessment. Participants received fludarabine 30 mg/m\hat{ }2/day and cyclophosphamide 500 mg/m\hat{ }2/day lymphodepletion chemotherapy for 3 consecutive days, from Day −5 to Day −3 followed by 2 rest days (Day −2 and Day −1), followed by a single infusion of brexucabtagene autoleucel at a target dose of 2×10\hat{ }6 anti-CD19 CAR T cells/kg or 1×10\hat{ }6 anti-CD19 CAR T cells/kg or a flat dose of 2×10⁸ or 1×10⁸, respectively, anti-CD19 CAR T cells in subjects >100 kg.

Certain inclusion criteria are common to all indications. 1) male or female 18 years of age or older at the time of signing the informed consent; 2) presence of toxicities due to prior therapy must be stable and recovered to Grade 1 or lower (except for clinically nonsignificant toxicities such as alopecia); 3) Eastern Cooperative Oncology Group (ECOG) performance status of 0 or 1; 4) adequate hematological function (unless lower values are attributable to underlying disease) as indicated by: absolute neutrophil count (ANC) ≥500/μL, platelet count ≥50,000/μL, hemoglobin level ≥8 g/dL; 5) absolute lymphocyte count ≥100/μL; 6) adequate renal, hepatic, pulmonary, and cardiac function defined as: creatinine clearance (as estimated by Cockcroft-Gault formula) ≥60 mL/min, serum alanine aminotransferase and aspartate aminotransferase levels≤2.5× upper limit of normal (ULN) or ≤5×ULN if documented liver involvement, total bilirubin levels≤1.5×ULN, except in subjects with Gilbert's syndrome, cardiac ejection fraction ≥50% and no evidence of pericardial effusion as determined by an echocardiogram (ECHO) or multigated acquisition scan (MUGA) and no clinically significant electrocardiogram (ECG) findings, no clinically significant pleural effusion, Baseline oxygen saturation >92% on room air; 7) the following washout periods must be satisfied prior to leukapheresis/enrollment: corticosteroid therapy at a pharmacologic dose (≥5 mg/day of prednisone or equivalent doses of other corticosteroids) must be avoided for 7 days before leukapheresis, BTK inhibitors (e.g., ibrutinib or acalabrutinib), must be avoided at least 1 week or 5 half-lives, whichever is shorter, before leukapheresis unless otherwise specified in the subprotocols, anti-neoplastic drugs used in previous therapy must be avoided within 1 week or 5 half-lives (whichever is shorter) prior to leukapheresis, systemic inhibitory/stimulatory immune checkpoint molecule therapy (e.g., ipilimumab, nivolumab, pembrolizumab, atezolizumab, OX40 agonists, 4-1BB agonists) must be avoided at least 3 half-lives prior to leukapheresis, alemtuzumab must be avoided at least 6 months prior to enrollment, PEG-asparaginase must be avoided at least 3 weeks prior to enrollment, cladribine and pentostatin must be avoided 3 months prior to enrollment, donor lymphocyte infusion within 28 days prior to enrollment, any treatment with immunosuppressive antibody used within 4 weeks prior to enrollment (e.g., anti-CD20, anti-tumor necrosis factor [TNF], anti-interleukin [IL] 6 or anti-IL6 receptor) unless this treatment is included in prior or bridging regimens, in which case a washout period of 7 days is required prior to leukapheresis; and 8) female subjects of childbearing potential must have a negative serum or urine pregnancy test (females who have undergone surgical sterilization or who have been postmenopausal for at least 2 years are not considered to be of childbearing potential).

Certain exclusion criteria are common to all indications. 1) prior CAR therapy or other genetically modified T-cell therapy; 2) prior treatment with any anti-CD19 therapy; 3) history of severe immediate hypersensitivity reaction attributed to aminoglycosides; 4) history of severe immediate hypersensitivity reaction to cyclophosphamide or fludarabine; 5) presence or suspicion of fungal, bacterial, viral, or other infection that is uncontrolled or requiring IV antimicrobials for management. Simple urinary tract infection and uncomplicated bacterial pharyngitis are permitted if responding to active treatment. Patients with a simple urinary tract infection and uncomplicated bacterial pharyngitis and responding to active treatment are eligible only if the patient satisfies the criteria of being afebrile (i.e., temperature lower than 38° C.) for at least 24 hours prior to the investigator confirming a patient's eligibility; 6) HIV-positive patients, unless taking appropriate anti-HIV medications, having an undetectable viral load by quantitative polymerase chain reaction (qPCR) and a CD4 count >200 cells/uL; 7) acute or chronic active hepatitis B or hepatitis C infection. Subjects with a history of hepatitis infection must have cleared their infection as determined by standard serological and genetic testing per current Infectious Diseases Society of America guidelines or applicable country guidelines; 8) presence of any indwelling line or drain (e.g., percutaneous nephrostomy tube, indwelling Foley catheter, biliary drain, or pleural/peritoneal/pericardial catheter) dedicated central venous access catheters, such as a Port-a-Cath or Hickman catheter, are permitted; 9) history or presence of detectable cerebrospinal fluid (CSF) malignant cells or brain metastases, unless otherwise specified in the substudy eligibility criteria; 10) history or presence of central nervous system (CNS) disorder, such as cerebrovascular ischemia/hemorrhage, dementia, cerebellar disease, or any autoimmune disease with CNS involvement, posterior reversible encephalopathy syndrome, or cerebral edema with confirmed structural defects by appropriate imaging. History of stroke or transient ischemic attack within 12 months before enrollment. Subjects with seizure disorders requiring active anticonvulsive medication; 11) presence of cardiac atrial or cardiac ventricular lymphoma involvement; 12) history of myocardial infarction, cardiac angioplasty or stenting, unstable angina, or other clinically significant cardiac disease within 12 months before enrollment; 13) requirement for urgent therapy due to tumor mass effects (e.g., blood vessel compression, bowel obstruction, or transmural gastric involvement); 14) presence of primary immunodeficiency; 15) history of autoimmune disease (e.g., Crohn's disease, rheumatoid arthritis, systemic lupus) resulting in end organ injury or requiring systemic immunosuppression/systemic disease modifying agents within the last 2 years; 16) history of deep vein thrombosis or pulmonary embolism requiring therapeutic anticoagulation within 6 months before enrollment; 17) any medical condition likely to interfere with assessment of safety or efficacy of study treatment; 18) history of severe immediate hypersensitivity reaction to any of the agents used in this study: 19) live vaccine ≤6 weeks before the planned start of the lymphodepleting chemotherapy regimen and anticipation of need for such a vaccine during the first 12 months after brexucabtagene infusion; 20) females who are pregnant or breastfeeding (because of the potentially dangerous effects of the preparative chemotherapy on the fetus or infant). Females who have undergone surgical sterilization or who have been postmenopausal for at least 2 years are not considered to be of childbearing potential; 21) not willing to practice birth control from the time of consent through 6 months after brexucabtagene autoleucel infusion; and 22) in the investigator's judgment, the subject is unlikely to complete all study-specific visits or procedures, including follow-up visits, or comply with the study requirements for participation.

Regarding Waldenstrom Macroglobulinemia, substudy specific inclusion criteria include: clinicopathological diagnosis of Waldenstrom Macroglobulinemia; ≥2 previous treatments for WM incl, a BTKi and chemotherapy with disease progression or no response; requiring treatment as per guidelines; and measurable disease (IgM level of >2 times the upper limit of normal). Regarding Waldenstrom Macroglobulinemia, substudy specific exclusion criteria include: allogeneic SCT; autologous SCT is allowed if ≥6 months have elapsed; and prior history CNS involvement (Bing-Neel syndrome), unless brain MRI and CSF are without pathology.

Regarding Richter Transformation, substudy specific inclusion criteria include: confirmed diagnosis of CLL based upon 2018 IWCLL criteria, with histologically confirmed Richter Transformation to a DLBCL subtype; at least 1 measurable site of disease based on 2014 Lugano Criteria; and R/R RT defined as 1 Primary refractory disease or relapsed after ≥1 lines of chemotherapy. Regarding Richter Transformation, substudy specific exclusion criteria include: prior allogeneic or autologous SCT <3 months prior to screening and/or <4 months prior to planned infusion of brexucabtagene autoleucel; and presence of active graft-versus-host disease following prior stem cell transplant.

Regarding Burkitt lymphoma/leukemia, substudy specific inclusion criteria include: histologically confirmed mature B-cell NHL Burkitt lymphoma/leukemia; R/R BL, defined as 1 primary refractory disease or relapsed after ≥1 lines of chemotherapy including an anthracycline; and measurable disease by radiological criteria or isolated bone marrow involvement. Regarding Burkitt lymphoma/leukemia, substudy specific exclusion criteria include: prior allogeneic SCT <3 months prior to screening, and patients with presence of active graft-versus-host disease following prior allogeneic stem cell transplantation.

Regarding Hairy Cell Leukemia, substudy specific inclusion criteria include: histologically confirmed Hairy Cell Leukemia; need for therapy based on: —neutrophils <1.0×10⁹/L, platelets <100×10⁹/L, hemoglobin <11 g/dL, and symptomatic splenomegaly or lymphadenopathy; and at least 2 prior systemic therapies, including at least a PNA and moxetumomab pasudotox if eligible and available.

In certain aspects, bridging therapy may be administered after leukapheresis and before lymphodepleting conditioning chemotherapy. In certain further aspects, bridging therapy must be completed ≥7 days or 5 half-lives before lymphodepleting conditioning chemotherapy.

At a health care provider's discretion, a subject with Richter transformation may receive a bridging therapy selected from the group consisting of Rituximab, Cyclophosphamide, Hydroxydaunorubicin hydrochloride, vincristine, and Prednisone (R-CHOP); Dose Adjusted Etoposide, Prednisone, Vincristine, Cyclophosphamide, Doxorubicin, and Rituximab (DA-EPOCH-R); Bruton Tyrosine Kinase inhibitor (BTKi) (BTKi)±VTX-2337; dexamethasone; and irradiation. Bridging therapy regimens for subjects with Richter transformation include those outlined in Table 10. Doses listed are embodiments only and may be adjusted for age, comorbidities, or per local or institutional guidelines.

TABLE 10
Bridging Therapy Regimens

| | | Timing and Washout |
|---|---|---|
| Type | Therapy Regimen | Requirements |
| R-CHOP | Rituximab 375 mg/m² IV on Day 1 | May be administered after |
| | Cyclophosphamide 750 mg/m² IV on | leukapheresis/enrollment |
| | Day 1 | Last dose must be completed |
| | Doxorubicin 50 mg/m² IV on Day 1 | at least 7 days or 5 half-lives, |
| | Vincristine 1.4 mg/m² (maximum dose | whichever is shorter, before |
| | 2 mg) IV on Day 1 | the start of lymphodepleting |
| | Prednisolone 40 mg/m² PO on Days 1 | chemotherapy |
| | to 5 | |
| R-ICE | Rituximab 375 mg/m² IV on Day 1 | |
| | Ifosfamide 5,000 mg/m²/3 IV on Days 2 | |
| | to 4 | |
| | Carboplatin (mg dose = 5 × AUC) IV on | |
| | Day 2 | |
| | Etoposide 100 mg/m² IV on Days 2 to 4 | |
| R-GEMOX | Rituximab 375 mg/m² IV on Day 1 | |
| | Gemcitabine 1,000 mg/m² IV on Day 2 | |
| | Oxaliplatin 100 mg/m² IV on Day 2 | |
| DA-EPOCH-R | Etoposide 50 mg/m²/day IV on Days 1 | |
| | to 4 | |
| | Doxorubicin 10 mg/m²/day IV on Days 1 | |
| | to 4 | |
| | Vincristine 0.4 mg/m²/day IV on Days 1 | |
| | to 4 | |
| | Cyclophosphamide 750 mg/m²/day IV | |
| | on Day 5 | |
| | Prednisone 60 mg/m²/BID PO on Days 1 | |
| | to 5 | |
| | Rituximab 375 mg/m² IV on Day 1 | |
| BTK inhibitor ± | BTK inhibitor as per local guidelines ± | |
| Venetoclax | Venetoclax as per local guidelines | |
| Dexamethasone | 40 mg daily for 4 days | |
| Irradiation | As per local guidelines | May be administered after |
| | | leukapheresis/enrollment |
| | | Last irradiation dose must be |
| | | completed at least 7 days |
| | | before the start of |
| | | lymphodepleting |
| | | chemotherapy |

Abbreviations: AUC, area under curve; BID, twice a day; BTK, Bruton's tyrosine kinase; DA-EPOCH-R, dose-adjusted etoposide, prednisone, vincristine, cyclophosphamide, doxorubicin, and rituximab; IV, intravenous; PO, oral; R-CHOP, rituximab with cyclophosphamide, doxorubicin, vincristine, and prednisolone; R-GEMOX, rituximab, gemcitabine, and oxaliplatin; .R-ICE, rituximab, ifosfamide, carboplatin, and etoposide

At a health care provider's discretion, a subject with Burkitt Lymphoma may receive a bridging therapy selected from the group consisting of Rituximab, Ifosfamide, Carboplatin, and Etoposide (R-ICE); Dose Adjusted Etoposide, Prednisone, Vincristine, Cyclophosphamide, Doxorubicin, and Rituximab (DA-EPOCH-R); Rituximab, Gemcitabine, and Oxaliplatin (R-GEMOX); cyclophosphamide, vincristine sulfate, doxorubicin hydrochloride, and dexamethasone (HyperCVAD); dexamethasone; and irradiation.

Bridging therapy regimens for subjects with Burkitt Lymphoma include those outlined in Table 11. Doses listed are embodiments only and may be adjusted for age, comorbidities, or per local or institutional guidelines.

TABLE 11
Bridging Therapy Regimens

| | | Timing and Washout |
|---|---|---|
| Type | Therapy Regimen | Requirements |
| R-ICE | Rituximab 375 mg/m² IV on Day 1 | May be administered after |
| } | Ifosfamide 5,000 mg/m² IV on Day 4 | leukapheresis/enrollment |
| | Carboplatin area under the curve 5, | Last dose must be completed |
| | dose = 5 × (25 + CrCl), capped at | at least 7 days or 5 half-lives, |
| | 800 mg, IV on Day 4 | whichever is shorter, before |
| | Etoposide 100 mg/m²/day IV on | the start of lymphodepleting |
| | Days 3 to 5 | chemotherapy |
| DA-EPOCH-R | Etoposide 50 mg/m²/day IV on | |
| | Days 1 to 4 | |
| | Doxorubicin 10 mg/m²/day IV on | |
| | Days 1 to 4 | |
| | Vincristine 0.4 mg/m²/day IV on | |
| | Days 1 to 4 | |
| | Cyclophosphamide 750 mg/m²/day | |
| | IV on Day 5 | |
| | Prednisone 60 mg/m²/BID PO on | |
| | Days 1 to 5 | |
| | Rituximab 375 mg/m² IV on Day 1 | |
| R-GEMOX | Rituximab 375 mg/m² IV on Days 1 | |
| | and 15 Gemcitabine 1,000 mg/m² IV | |
| | on Day 1 | |
| | Oxaliplatin 100 mg/m² IV on Day 1 | |
| Hyper-CVAD | Hyperfractionated cyclophosphamide | |
| | 300 mg/m² IV every 12 hours for | |
| | 6 doses on Days 1 to 3, with sodium | |
| | mercaptoethanesulfonate 600 mg/m² | |
| | daily via continuous infusion on | |
| | Days 1 to 3 | |
| | Vincristine 2 mg IV on Days 4 and | |
| | 11 | |
| | Doxorubicin 50 mg/m² IV over | |
| | 24 hours on Day 4 | |
| | Dexamethasone 40 mg PO or IV | |
| | daily on Days 1 to 4 and Days 11 to | |
| | 14 | |
| Dexamethasone | 40 mg daily for 4 days | |
| Irradiation | As per local guidelines | May be administered after |
| | | leukapheresis/enrollment |
| | | Last irradiation dose must be |
| | | completed at least 7 days |
| | | before the start of |
| | | lymphodepleting |
| | | chemotherapy |

Abbreviations: BID, twice a day; CrCl, creatinine clearance; DA-EPOCH-R, dose-adjusted etoposide, prednisone, vincristine, cyclophosphamide, doxorubicin, and rituximab; hyper-CVAD, hyperfractionated cyclophosphamide, vincristine, doxorubicin, and dexamethasone; IV, intravenous; PO, oral; R-GEMOX, rituximab with gemcitabine and oxaliplatin; R-ICE, rituximab with ifosfamide, carboplatin, and etoposide.

At a health care provider's discretion, a subject with Waldenstrom Macroglobulinemia may receive a bridging therapy which is ibrutinib.

### Example 4

Brexucabtagene autoleucel (brexu-cel) is an autologous anti-CD19 chimeric antigen receptor (CAR) T-cell therapy approved for relapsed/refractory mantle cell lymphoma (R/R MCL). After 3 years of follow-up in the pivotal Phase 2 ZUMA-2 study, 91% of patients with MCL progressing on BTK inhibitors responded to brexu-cel therapy with a median duration of response (DOR) of 28.2 months. This example presents data assessing patient, product, and PK characteristics in ZUMA-2 in ongoing responders (patients in response at the 24-month assessment), relapsed responders (patients who initially responded, but relapsed prior to the 24-month assessment), and nonresponders.

At the median follow-up of 35.6-months, 68 patients received brexu-cel with 91% of patients (n=62) achieving a response (CR or PR) and 9% of patients (n=6) with no response. Of the responders, 29 had an ongoing response at their 24-month assessment (28 CR and 1 PR) (15 CR and 14 PR). Ongoing responders had lower baseline median tumor burden (sum of product diameters, 935 vs. 4861 mm²) and a higher frequency of Eastern Cooperative Oncology Group (ECOG) performance status 0 (79% vs. 59%) compared with relapsed responders. A smaller proportion of ongoing responders received prior platinum therapy (10% vs. 41%) or bridging therapy (21% vs. 52%) compared to relapsed responders.

Median DOR was 47.1 months (95% CI, 36.5 months-not estimable) and 5.0 months (95% CI, 2.2-8.3) in ongoing and relapsed responders, respectively. Medians for DOR were similar for patients with high baseline lactate dehydrogenase vs patients with low baseline LDH, regardless or response status.

Medians for peak and area under the curve CAR T-cell expansion were about twice as large in ongoing responders vs relapsed responders. Product characteristics were largely similar among ongoing and relapsed responders with a modest increase in the median total number of naïve-like infused chemokine receptor 7 (CCR7)-positive T cells observed in ongoing vs relapsed responders.

In summary, ongoing responders had lower tumor burden, less use of prior platinum and bridging therapies and higher CAR T-cell expansion, suggesting that patients with lower overall disease burden or less prior chemotherapy may have a greater likelihood for durable response with brexu-cel. However, ongoing responses were also observed in patients with high-risk disease characteristics.

### INTRODUCTION

Although Bruton tyrosine kinase (BTK) inhibition provided a paradigm shift in mantle cell lymphoma (MCL) therapy, these agents have not proven to be curative. In patients with relapsed/refractory (R/R) MCL treated with BTK inhibitors, median progression-free survival (PFS) values in the range of 13-33 months have been reported and discontinuation of therapy due to progression or intolerance is common. The clinical benefits of BTK inhibitors in patients with high-risk features including first progression within 24 months of initial diagnosis (POD24), TP53 aberrations, elevated lactate dehydrogenase (LDH) at progression, and blastoid variants are even more limited. Moreover, outcomes of post-BTK inhibitor salvage therapy are poor, with median overall survival (OS) times as short as 2.5-8.4 months reported. In addition, the BTKi ibrutinib has recently been withdrawn from its MCL indication in the United States due to toxicity concerns. Thus, an unmet need remains for better treatment options for patients with R/R MCL.

Chimeric antigen receptor (CAR) T-cell therapy represents another landmark advance in the treatment of hematologic malignancies. Brexucabtagene autoleucel (brexu-cel, previously known as KTE-X19) is an autologous anti-CD19 CAR T-cell therapy approved in the United States for the treatment of adults with R/R MCL and in the European Union for the treatment of adults with R/R MCL after 22 prior systemic treatments, including a BTK inhibitor. Accelerated approval was based on results from the pivotal, single-arm, multicenter Phase 2 ZUMA-2 (NCT02601313) study of brexu-cel therapy in patients with R/R MCL. All patients had progressed on BTK inhibitor therapy (62% refractory), and many had high-risk disease.

With a median follow-up of 35.6 months in ZUMA-2, the overall response rate (ORR) was 91% (95% CI, 81.8-96.7), including a 68% complete response (CR) rate, in 68 treated patients. The median duration of response (DOR) among responders was 28.2 months (95% CI, 13.5-47.1), and was considerably longer in patients achieving CR (46.7 months) than in those with partial response (PR; 2.2 months). Median PFS and OS were 25.8 months (95% CI, 9.6-47.6) and 46.6 months (95% CI, 24.9-not estimable [NE]), respectively. The most common Grade ≥3 treatment-emergent adverse events (TEAEs) at median 12.3-month follow-up were cytopenias (94%) and infections (32%) with Grade ≥3 cytokine release syndrome (CRS) and neurologic events occurring in 15% and 31% of patients, respectively. Similar real-world results were observed from the US Lymphoma CAR T Consortium which found that brexu-cel demonstrated a 90% overall response rate and an 82% complete response rate in 168 patients with R/R MCL who received brexu-cel treatment in the standard-of-care setting. In addition, the study found that 8% and 32% of these patients experienced Grade 23 CRS and neurotoxicity following brexu-cel infusion, respectively.

Understanding the association between patient, product, and pharmacokinetic characteristics and durable response to brexu-cel in patients with R/R MCL could inform up-front patient selection, thereby maximizing benefit. This study examined the association of these factors with long-term response to brexu-cel in ZUMA-2.

### Methods

### Study Design and Patients

Detailed methodology for the multicenter, single-arm ZUMA-2 (NCT02601313) was previously described. Briefly, patients were ≥18 years of age and had histologically confirmed MCL that was relapsed or refractory to 1 to 5 previous MCL regimens including anthracycline- or bendamustine-containing chemotherapy, an anti-CD20 monoclonal antibody, and BTK inhibitor therapy with either ibrutinib or acalabrutinib. All patients underwent leukapheresis, after which those with high disease burden could receive bridging therapy with steroids or BTK inhibitors at the investigator's discretion.

Conditioning chemotherapy consisted of once-daily intravenous (IV) fludarabine 30 mg/m² and cyclophosphamide 500 mg/m² on Days −5, −4, and −3. A single IV infusion of brexu-cel was administered at a target dose of 2×10⁶ CAR T cells/kg on Day 0. All patients provided written informed consent and the trial was conducted in accordance with the principles of the Declaration of Helsinki.

### End Points and Assessments

The present analysis examined baseline patient and disease characteristics, product characteristics, subsequent therapies, and pharmacological outcomes by response status at 24 months post-brexu-cel infusion as assessed by the independent radiology review committee using Lugano classification. Ongoing responders were defined as those with an ongoing CR or PR at their 24-month assessment. Relapsed responders were defined as those with a previous response who had either relapsed, proceeded to subsequent anticancer therapy (including SCT), or died by any cause prior to their 24-month assessment. Nonresponders were patients who had not achieved a response. DOR was assessed in both responder groups. Levels of transduced anti-CD19 CAR T cells in blood were measured by quantitative polymerase chain reaction. T-cell phenotypes were assessed by multicolor flow cytometry using protocols and antibodies that were previously described.

### Statistical Analysis

All subgroup analyses were post-hoc, exploratory analyses with descriptive statistics provided. Time-to-event end points were analyzed using the Kaplan-Meier methodology.

### Results

### Patients

Of the 74 patients enrolled and leukapheresed in ZUMA-2, brexu-cel was successfully manufactured for 71 (95.9%), and 68 patients (91.9%) received brexu-cel. As of Jul. 24, 2021, the median follow-up time was 35.6 months (range, 25.9-56.3), 62 patients (91.2%) achieved a best response of CR or PR and 6 patients (8.8%) did not achieve a response. Among the 62 responders. 29 (47%; 28 CR and 1 PR) were in ongoing response at their 24-month assessment (ongoing responders). 29 (47%; 15 CR and 14 PR) had relapsed prior to their 24-month assessment (relapsed responders), and 4 did not reach or missed their 24-month assessment and were excluded from this analysis.

### Patient Characteristics

Most baseline characteristics were similar across ongoing responders, relapsed responders, and non-responders. However, ongoing responders had an approximately fourfold lower tumor burden at baseline (median SPD 935 vs. 4861 mm²), were less likely to have received prior platinum therapies (10% vs. 41%) or bridging therapy (21% vs. 52%), less likely to have POD24 (33% vs 66%), and were more likely to have Eastern Cooperative Oncology Group (ECOG) performance status of 0 (79% vs. 59%) at baseline compared with relapsed responders. Similar proportions of ongoing responders and relapsed responders received prior bendamustine (45% and 52%), prior anthracycline (76% and 72%), and prior proteasome inhibitors (41% and 34%), respectively. These trends generally held true when comparing only ongoing responders with CR to relapsed responders with CR.

The median number of prior therapies was 3 in all subgroups. Ibrutinib was the most common prior BTKi received in all subgroups and was the most common last prior therapy received in all subgroups; though it was more common among ongoing responders (93% total; 66% as last prior therapy) than in relapsed responders (79% total; 41% as last prior therapy) or non-responders (67% total; 33% as last prior therapy). Prior acalabrutinib was received by 28% of ongoing responders, 21% of relapsed responders, and 33% of non-responders; and was the last prior therapy for 14% of ongoing responders, 14% of relapsed responders, and 17% of non-responders). The median time from last prior therapy to brexu-cel infusion was 63 days (range, 26-748) for ongoing responders, 63 days (range, 22-443) for relapsed responders, and 136 days (range, 29-642) for non-responders.

### Efficacy

Among patients who had achieved CR, the median DOR was not reached (95% CI, 46.7-NE) in ongoing responders (n=28) and was 8.3 months (95% CI, 5.0-13.6) in relapsed responders (n=15). At data cutoff, 22 of the 28 ongoing responders with CR (79%) were in ongoing response without subsequent therapy, 1 (4%) proceeded to new anticancer therapy, 2 (7%) had disease progression, and 3 (11%) died. At data cutoff, none of the 15 relapsed responders with CR were in ongoing response without subsequent therapy, 2 (13%) proceeded to subsequent SCT, 1 (7%) proceeded to new anticancer therapy, 12 (80%) had disease progression, and none died.

Among patients who achieved any response (CR or PR), median DOR was 47.1 months (95% CI, 36.5-NE) in ongoing responders (n=29) and 5.0 months (95% CI, 2.2-8.3) in relapsed responders (n=29). Median time to response was 1 month for both ongoing responders (range. 0.9-3.1; n=29) and relapsed responders (range, 0.8-1.7; n=29) and median time to CR was 3 months for both ongoing responders (range, 0.9-35.1; n=28) and relapsed responders (range, 0.8-9.0; n=15). As previously reported, MRD-negativity at 6 months correlated with longer median DOR, PFS, and OS.

Given that elevated LDH is a known poor prognostic indicator for patients with R/R MCL. DOR was assessed in patients with high and low baseline LDH. The median DOR among ongoing responders with high baseline LDH (≥1 ULN; n=13) and low baseline LDH (<1 ULN; n=14) was 47.1 (95% CI, 24.8-NE) and 46.7 months (95% CI, 24.4-NE), respectively. For relapsed responders with high baseline LDH (n=11) and low baseline LDH (n=18), the median DOR was 3.6 months (95% CI, 1.0-13.5) and 5.4 months (95% CI, 2.2-8.6), respectively.

Subsequent anticancer therapies were received by 1/29 (3%). 20/29 (69%), and 3/6 (50%) patients in the ongoing responder, relapsed responder, and non-responder subgroups, respectively. The most common of these were radiotherapy, rituximab, dexamethasone, lenalidomide, and venetoclax.

### Product and Pharmacokinetic Characteristics

Product characteristics were generally similar between ongoing and relapsed responders. Numerical differences were observed among median CD4/CD8 ratios for ongoing responders, relapsed responders, and non-responders at 0.86 (range, 0.27-2.06), 0.64 (range. 0.04-3.73), and 0.41 (range. 0.25-0.73), respectively. Although the total number of CAR T cells infused was similar across subgroups, the total number of CCR7+ T-cells infused was modestly increased among ongoing responders relative to the other subgroups, with median levels (×10⁶) of 119.8 (range, 37.0-249.9), 89.4 (range, 6.1-353.4), and 88.2 (range, 39.9-150.3), respectively.

Median peak CAR T-cell levels were 102.4 cells/μl (range, 0.3-2242.6) and 62.7 cells/μl (range. 1.6-2589.5) in ongoing responders and relapsing responders, respectively. Similarly, median CAR T-cell area under the curve (from Day 0 To Day 28; AUC₀₋₂₈) values were 1487.0 cells/μl×days (range, 3.8-16700) and 775.8 cells/μl×days (range, 19.0-27200), in these subgroups, respectively. Non-responders had the lowest median CAR T-cell peak (5.9 cells/μl; range, 0.2-95.9) and AUC₀₋₂₈ values (24.7 cells/μl×days; range, 1.8-1089.1) of any subgroup.

Differences in peripheral blood T-cell phenotypes at Day 7 were observed among subgroups including a significantly higher median proportion of differentiated CD8+CD27−CD28+ cells and CD8+ CCR7− CD45RA+ CD27− CD28+ terminally differentiated effector memory cells in combined relapsed responders and non-responders compared with ongoing responders (P=0.0023 and P=0.0052, respectively; Table 12). Ongoing responders had significantly more peripheral CD4+ T cells that maintain juvenile CD27+ expression than combined relapsed responders and non-responders (P=0.03) and exhibited a trend toward higher levels of activated CD8 effector memory T cells (P=0.057; Table 12).

TABLE 12

| Phenotypes of peripheral T-cells | Phenotypes of peripheral blood T-cells |
|---|---|
| associated with ongoing response | associated with relapse and/or nonresponse |
| CD27+CD28− CD4+ CD3+ T cells | CD27−CD28+ CD8+ CAR− CD3+ T cells |
| CD27+CD28− CCR7−CD45RA− CD4+ CD3+ | CD27−CD28+ CCR7−CD45RA− CD8+ CAR− |
| T cells | CD3+ T cells |
| PD1+TIM3+ CCR7−CD45RA+ CD4+ CAR+ | CD27−CD28+ CCR7−CD45RA+ CD8+ |
| CD3+ T cells | CAR− CD3+ T cells |
| CD27+CD28− CD4+ CAR− CD3+ T cells | CD27−CD28+ CD8+ CD3+ T cells |
| CD27+CD28− CCR7−CD45RA+ CD4+ CD3+ | CD27−CD28+ CCR7−CD45RA+ CAR− CD3+ |
| T cells | T cells |
| PD1+ CCR7+CD45RA− CD8+ CD3+ T cells | CD27−CD28+ CCR7−CD45RA− CD8+ CD3+ |
| | T cells |
| CD27+CD28− CCR7−CD45RA− CD4+ CAR+ | CD27−CD28+ CCR7−CD45RA+ CD8+ CD3+ |
| CD3+ T cells | T cells |
| LAG3+PD1+TIM3+ CCR7−CD45RA+ CD4+ | CD27+CD28+ CCR7−CD45RA+ CD8+ |
| CAR+ CD3+ T cells | CAR− CD3+ T cells |
| LAG3+TIM3+ CCR7−CD45RA+ CD4+ | CD27−CD28− CCR7−CD45RA− CAR+ CD3+ |
| CAR+ CD3+ T cells | T cells |
| PD1+ CCR7+CD45RA− CAR+ CD3+ T cells | CD27−CD28+ CCR7−CD45RA+ CD3+ T |
| | cells |
| CD27+CD28− CD4+ CAR+ CD3+ T cells | CD27−CD28+ CAR− CD3+ T cells |
| PD1+ CCR7+CD45RA− CD8+ CAR− CD3+ T | CCR7+CD45RA+ CD8+ CAR− CD3+ T cells |
| cells | |
| CD27+CD28− CCR7+CD45RA− CD4+ | CD27−CD28+ CCR7−CD45RA− CAR− CD3+ |
| CAR+ CD3+ T cells | T cells |
| LAG3+ CCR7−CD45RA+ CD4+ CAR+ | CD27−CD28+ CCR7−CD45RA+ CD4+ CAR− |
| CD3+ T cells | CD3+ T cells |
| LAG3+PD1+ CCR7−CD45RA+ CD4+ CAR+ | CD27−CD28+ CCR7−CD45RA+ CD4+ CD3+ |
| CD3+ T cells | T cells |
| PD1+ CCR7+CD45RA− CAR− CD3+ T cells | CD27−CD28+ CCR7−CD45RA− CAR+ CD3+ |
| | T cells |
| | LAG3+ CCR7+CD45RA+ CD8+ CD3+ T |
| | cells |
| | LAG3+PD1+CCR7+CD45RA+ CD8+ CD3+ |
| | T cells |
| | CD27+CD28+ CCR7−CD45RA+ CD8+ |
| | CD3+ T cells |
| | CD27−CD28+ CD4+ CAR+ CD3+ T cells |
| | CD27+CD28+ CCR7−CD45RA+ CAR− |
| | CD3+ T cells |
| | CD27−CD28+ CD3+ T cells |
| | CD27−CD28+ CCR7−CD45RA− CD8+ CAR+ |
| | CD3+ T cells |
| | CD27−CD28+ CCR7−CD45RA− CD3+ T cells |
| | CD27−CD28+ CCR7−CD45RA− CD4+ CAR+ |
| | CD3+ T cells |
| | CD27−CD28− CCR7−CD45RA− CD8+ CAR+ |
| | CD3+ T cells |
| | CD27−CD28+ CD4+ CD3+ T cells |
| | CCR7+CD45RA− CD8+ CAR− CD3+ T cells |
| | CD27−CD28+ CD8+ CAR+ CD3+ T cells |
| | CD27−CD28+ CCR7+CD45RA+ CD4+ |
| | CAR− CD3+ T cells |
| | LAG3+TIM3+ CCR7+CD45RA− CD4+ |
| | CAR− CD3+ T cells |
| | CD27−CD28+ CAR+ CD3+ T cells |
| | CCR7+CD45RA+ CD8+ CD3+ T cells |
| | CD27−CD28+ CD4+ CAR− CD3+ T cells |
| | TIM3+ CD8+ CAR+ CD3+ T cells |
| | LAG3+PD1+TIM3+ CCR7+CD45RA+ |
| | CD4+ CD3+ T cells |
| | LAG3+TIM3+ CCR7+CD45RA+ CD4+ |
| | CD3+ T cells |
| | CD27+CD28+ CCR7−CD45RA+ CD3+ T |
| | cells |

### Discussion

This analysis identifies associations between patient, disease, product, and/or pharmacokinetic characteristics with durable response to brexu-cel in patients with R/R MCL treated in ZUMA-2. The analysis identified a 47% rate of ongoing response at 24 months post-infusion. Considering the poor prognosis and limited survival associated with the failure of BTK inhibitors in this setting, these results continue to support brexu-cel as a favorable treatment option in this disease setting. Interestingly, the median DOR for relapsed responders was only 5 months, whereas for ongoing responders it was 47.1 months, suggesting that patients who remained in ongoing response after 24 months had favorable long-term outcomes. Notably, the marked differences in the Kaplan-Meier DOR curves for ongoing versus relapsed responders between 6 and 12 months suggest that these earlier timepoints may predict long-term responses.

Because CAR T-cell therapy is a relatively recent approach to treating patients with hematologic malignancies, factors associated with response are subject to ongoing investigation. These factors may be specific to a given CAR T-cell product and/or malignancy, and conclusive data have yet to emerge. Patient characteristics such as baseline tumor burden and LDH levels, as well as T-cell phenotypes such as the proportion of memory T cells and CD4+/CD8+ T-cell ratios have been correlated with response or response durability to CD-19-targeting CAR T-cell therapy. More recently, tumor immune contexture was suggested to be a determinant of CAR T-cell efficacy and may play a role in response durability in this analysis as well.

In ZUMA-2, tumor burden as measured by SPD at baseline was notably lower (935 vs. 4861 mm²) in ongoing responders than relapsed responders. A similar correlation between SPD and outcomes has been reported in another study of CAR T-cell therapy in B-cell malignancies. Prior platinum therapy and bridging therapy were less frequent in ongoing responders compared with relapsed responders, and ongoing responders were more likely to have a better ECOG performance status than relapsed responders. Collectively, these data suggest that among patient and disease characteristics, higher tumor burden at baseline correlated with a higher risk of relapse by 24 months. In contrast, the known risk factor of high baseline LDH levels (≥upper limit of normal) was not associated with inferior response durability, as patients with low and high baseline LDH within each subgroup (ongoing or relapsed responders) had similar median DORs. Thus, brexu-cel was associated with durable response irrespective of LDH status at baseline.

Ibrutinib was more frequently received as a prior therapy and as the last prior therapy in ongoing responders than in relapsed responders or non-responders. Interestingly, the same phenomenon was not observed with prior acalabrutinib therapy as similar proportions of ongoing responders, relapsed responders, and non-responders received acalabrutinib as a prior therapy or as the last prior therapy. In preclinical studies, ibrutinib has been shown to improve CAR T-cell persistence and efficacy, possibly improving T-cell function and expansion through its off-target inhibition of inducible T-cell kinase. In addition, a time-limited combination of ibrutinib and tisagenlecleucel demonstrated encouraging efficacy (ORR of 90%) in a small population of patients with R/R MCL (N=20) in the TARMAC trial. Additional clinical studies are needed to further understand the impact prior ibrutinib may have on the long-term efficacy of brexu-cel therapy.

In a previous analysis of ZUMA-2, prior bendamustine use within 6 months of apheresis was associated with a poorer pharmacokinetic profile and reduced product doubling time of infused CAR T cells, suggesting that the timing of bendamustine use may attenuate T-cell fitness; though small sample sizes limit interpretation of this finding. In the current analysis, the rates of prior bendamustine use were similar among ongoing responders and relapsed responders; however, due to small samples sizes, we were unable to assess whether the timing of prior bendamustine impacted the durability of response.

CAR T products derived from patients with differing T-cell subsets can reasonably be expected to vary in their effects. In ZUMA-2. T-cell phenotype data were available for the majority of patients, allowing a comparison of T-cell subsets in ongoing responders versus combined relapsed responders/non-responders. Increases in median number of CCR7+ cells and CD27+ peripheral T cells among ongoing responders suggest that continuous memory T-cell differentiation may play a role in achievement of durable response.

In a previous study of axicabtagene ciloleucel (ZUMA-1), peak CAR T-cell levels and CAR T-cell AUC were found to correlate with durable (24-month) response. The present analysis identified a similar trend in patients with R/R MCL treated with brexu-cel in ZUMA-2. Both median peak and AUC levels of CAR T cells were approximately twice as high in ongoing responders, indicating that robust CAR T-cell expansion may contribute to achieving a durable response. These findings are consistent with the results from ZUMA-7 even though this effect has not been observed in other CAR T studies with different CAR T products (e.g., JULIET). Factors specific to the disease context, tumor characteristics including the microenvironment as well as patient baseline characteristics as well as product characteristics may preclude extrapolation or generalization.

These results demonstrate durable responses for patients with R/R MCL treated with brexu-cel, including those with high-risk disease features who would otherwise have a poor prognosis. Several factors that appeared to be associated with 2-year ongoing response (i.e., ECOG performance status. SPD, prior platinum, use of bridging therapy) relate to the advanced or aggressive nature of the disease, suggesting more optimal use of brexu-cel may be earlier in the course of the disease. Consistent with some, but not all prior CAR T studies, the degree of CAR T-cell expansion was associated with the durability of response. These findings together with those of further investigations should aid in identifying the patients likely to derive the greatest benefit from brexu-cel in this difficult-to-treat malignancy.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the disclosure.

1.-69. (canceled)

70. A method for treating a cancer in a subject in need thereof wherein the subject has previously been administered a first T cell product comprising autologous T cells expressing an anti-CD19 chimeric antigen receptor (CAR), further wherein a peripheral blood sample has been taken from the subject after administration of the first T cell product, comprising (a) measuring the level of CD8+CD27−CD28+ T-cells in the blood sample, and (b) if the level of CD8+CD27−CD28+ T-cells in the blood sample is elevated, administering to the subject a second T cell product.

71. (canceled)

72. The method of claim 70, wherein the first T cell product comprises CD4+ and CD8+ T cells that have been prepared from peripheral blood mononuclear cells (PBMCs) by positive enrichment and consequent partial or complete depletion of circulating cancer cells.

73. The method of claim 72, wherein the CD4+ and CD8+ T cells have been activated with anti-CD3 and anti-CD28 antibodies in the presence of IL-2, and then transduced with a replication-incompetent viral vector encoding, a chimeric antigen receptor (CAR) comprising an anti-CD19 single-chain variable fragment (scFv), CD28 and CD3-zeta domains.

74. The method of claim 70, wherein the cancer is selected from the group consisting of mantle cell lymphoma (MCL), B cell ALL, Waldenstrom Macroglobulinemia, Richter Transformation, Burkitt Lymphoma, and Hairy Cell Leukemia.

75. The method of claim 74, wherein the cancer is MCL.

76. The method of claim 70, wherein the blood sample has been taken from the subject between day 5 and 9 after administration of the first T cell product.

77. The method of claim 76, wherein the blood sample has been taken from the subject between day 6 and 8 after administration of the first T cell product.

78. The method of claim 77, wherein the blood sample has been taken from the subject on Day 7 after administration of the first T cell product.

79. The method of claim 70, wherein the blood sample has been taken from the subject between day 12 and 16 after administration of the first T cell product.

80. The method of claim 79, wherein the blood sample has been taken from the subject between day 13 and 15 after administration of the first T cell product.

81. The method of claim 80, wherein the blood sample has been taken from the subject on Day 14 after administration of the first T cell product.

82. The method of claim 70, wherein an elevated level of CD8+CD27−CD28+ T-cells for the subject is determined by comparison to other subjects who have received a comparable T cell product and have had a peripheral blood sample taken on the same day after T cell product administration.

83. (canceled)

84. The method of claim 70, wherein the second T cell product is selected from the group consisting of an autologous CD19/CD20 bi-cistronic T-cell product and an allogenic T-cell product.

85. (canceled)

86. (canceled)

87. A method for monitoring a subject who has previously been administered a first T cell product comprising autologous T cells expressing an anti-CD19 chimeric antigen receptor (CAR), comprising
(a) taking a blood sample from the subject after administration of the first T cell product,
(b) measuring the level of CD8+CD27−CD28+ T-cells in the blood sample, and
(c) prescribing a course of treatment based on the level of CD8+CD27−CD28+ T-cells in the blood sample, wherein if the level of CD8+CD27−CD28+ T-cells is elevated then a second T cell product is administered.

88-90. (canceled)
